(19) United States
(12) Patent Application Publication
(10) Pub. No.: US 2014/0068814 A1
(43) Pub. Date: Mar. 6, 2014

(54) METHOD OF IMPROVING ABIOTIC STRESS TOLERANCE OF PLANTS AND PLANTS GENERATED THEREBY

(71) Applicant: A.B. Seeds Ltd., Lod (IL)

(72) Inventors: Orly NOIVIRT-BRIK, Givataim (IL); Rudy Maor, Rechovot (IL); Amir Avniel, Tel-Aviv (IL)

(73) Assignee: A.B. Seeds Ltd., Lod (IL)

(21) Appl. No.: 14/016,383

(22) Filed: Sep. 3, 2013

Related U.S. Application Data
(60) Provisional application No. 61/696,250, filed on Sep. 3, 2012.

(51) Int. Cl. C12N 15/82

(52) U.S. Cl. CPC C12N 15/8273
USPC 800/285; 800/298; 435/419

(57) ABSTRACT

A method of improving abiotic stress tolerance of a plant is provided. The method comprising genetically modifying the plant to express miRNA167 in an abiotic stress responsive manner, wherein a level of expression of total miR167 under the abiotic stress conditions is selected not exceeding 10 fold compared to same in the plant when grown under optimal conditions, thereby improving abiotic stress tolerance of the plant.

### RELATED APPLICATION

This application claims the benefit of priority under 35 USC 119(e) of U.S. Provisional Patent Application No. 61/696,250 filed Sep. 3, 2012, the contents of which are incorporated herein by reference in their entirety.

### SEQUENCE LISTING STATEMENT

The ASCII file, entitled 57315SequenceListing.txt, created on Sep. 1, 2013, comprising 173,132 bytes, submitted concurrently with the filing of this application is incorporated herein by reference.

### FIELD AND BACKGROUND OF THE INVENTION

The present invention, in some embodiments thereof, relates to a method of improving abiotic stress tolerance of plants and plants generated thereby.

Abiotic stresses including drought are serious threats to the sustainability of crop yields accounting for more crop productivity losses than any other factor in rain fed agriculture.

Among the abiotic stresses that limit plant growth, drought is the most complex and devastating on a global scale.

Drought is an increasingly important constraint of crop productivity and stability world-wide due to climate change. With continuing yield losses due to an expected water scarcity, crops with greater ability to adapt to reduced water use are needed to cope with increasingly severe drought conditions.

As an example, in 2012, America's corn stocks were at their lowest in 20 years due to one of the hottest summers on record. The impact could affect the production of ethanol, which is created using the corn harvest in the U.S. That could in turn mean an increase in carbon dioxide emissions, as well as a further increase in droughts from climate change. Likewise, in 2010, bean yields in parts of Michigan were reduced by 50% when summer rainfall was reduced by over 60%.

Thus, with a growing world population, increasing demand for food, fuel and fiber, and a changing climate, agriculture faces unprecedented challenges. Farmers are seeking advanced, biotechnology-based solutions to enable them to obtain stable high yields and give them the potential to reduce irrigation costs or to grow crops in areas where potable water is a limiting factor.

Research focuses on the development of genotypes with resistance to intermittent and terminal drought in various crops. Traits associated with drought tolerance have been identified for some, but the work is low and cumbersome requiring long selection steps for each crop. Therefore, transgenic crops are being developed which can endure abiotic stress conditions.

### SUMMARY OF THE INVENTION

According to an aspect of some embodiments of the present invention there is provided a method of improving abiotic stress tolerance of a plant, the method comprising genetically modifying the plant to express miRNA167 in an abiotic stress responsive manner, wherein a level of expression of total miR167 under the abiotic stress conditions is selected not exceeding 10 fold compared to same in the plant when grown under optimal conditions, thereby improving abiotic stress tolerance of the plant.

According to some embodiments of the invention, genetically modifying the plant to express miRNA167 is effected by expressing within the plant an exogenous polynucleotide encoding miR167.

According to some embodiments of the invention, the exogenous polynucleotide is expressed under an abiotic stress-responsive (e.g., drought)-responsive promoter.

According to some embodiments of the invention, the abiotic stress-responsive promoter is selected from the group consisting of OsABA2, OsPrx, Wcor413, Lip5, rab16A, XVSAP1 and OsNAC6.

According to some embodiments of the invention, the abiotic stress-responsive promoter is OsNAC6.

According to some embodiments of the invention, the level of expression of total miR167 under optimal conditions is as that of miR167 in a non-genetically modified plant of the same species and growth conditions.

According to some embodiments of the invention, the level of expression of total miR167 under the abiotic stress does not exceed 8 fold as compared to same in the plant when grown under the optimal conditions.

According to some embodiments of the invention, the level of expression of total miR167 under the abiotic stress does not exceed 5 fold as compared to same in the plant when grown under the optimal conditions.

According to some embodiments of the invention, the level of expression of total miR167 under the abiotic stress does not exceed 3 fold as compared to same in the plant when grown under the optimal conditions.

According to some embodiments of the invention, the level of expression of total miR167 under the abiotic stress does not exceed 2 fold as compared to same in the plant when grown under the optimal conditions.

According to some embodiments of the invention, the level of expression of total miR167 under the abiotic stress does not exceed 1.4-2 fold as compared to same in the plant when grown under the optimal conditions.

According to some embodiments of the invention, the level of expression of total miR167 under the abiotic stress does not exceed 1.7-2 fold as compared to same in the plant when grown under the optimal conditions.

According to some embodiments of the invention, the method further comprises growing the plant under the abiotic stress.

According to some embodiments of the invention, the abiotic stress is selected from the group consisting of salinity, water deprivation, low temperature, high temperature, heavy metal toxicity, anaerobiosis, nutrient deficiency, nutrient excess, atmospheric pollution and UV irradiation.

According to some embodiments of the invention, the water deprivation comprises drought.

According to some embodiments of the invention, the drought is intermittent drought.

According to some embodiments of the invention, the drought is terminal drought.

According to an aspect of some embodiments of the present invention there is provided a plant or a plant cell genetically modified to express miR167, wherein expression of the miRNA167 in the plant cell is abiotic stress responsive and further wherein a level of expression of total miR167 in the plant cell under the abiotic stress does not exceed 10 fold as compared to same in a plant when grown under optimal conditions.

According to an aspect of some embodiments of the present invention there is provided a plant or plant cell generated according to the method described herein.

According to some embodiments, is provided a method of improving abiotic stress tolerance of a grafted plant, the method comprising providing a scion that does not transgenically express miR167 and a plant rootstock that transgenically expresses a miR167 in an abiotic stress responsive manner, wherein a level of expression of total miR167 in the transgenic plant root stock under the abiotic stress conditions is selected not exceeding 10 fold compared to same plant rootstock when grown under optimal conditions, thereby improving abiotic stress tolerance of the grafted plant. In some embodiments, the plant scion is non-transgenic. Several embodiments relate to a grafted plant exhibiting improved abiotic stress tolerance, comprising a scion that does not transgenically express miR167 and a plant rootstock that transgenically expresses a miR167. In some embodiments, the plant root stock transgenically expresses a miR167 in a stress responsive manner. In some embodiments, the level of expression of total miR167 by the transgenic root stock under the abiotic stress does not exceed 10 fold as compared to same root stock when grown under the optimal conditions. In some embodiments, the level of expression of total miR167 by the transgenic root stock under the abiotic stress does not exceed about 1.4, 1.7, 2, 3, 4, 5, 6, 7, 8, or 9 fold as compared to same root stock when grown under the optimal conditions. In some embodiments the grafted plant is a tomato or an eggplant.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, examples of methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the figures in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B are photographs showing a significant increase in yield for miR167 transgenic plants grown under drought conditions as compared to wild-type plants. The photographs were taken 4.5 (1A) or 5 (1B) months following seeding while the plants were grown as described in the Examples section.

FIGS. 2A-B show down-regulation of miR167 target genes, ARF6 and ARF8, in transgenic tomato plants expressing miR167FIG. 2A—Sly-ARF6 down-regulation compared to control (transgenic empty vector), p-value=0.022, fold change of 1.87, FIG. 2B Sly-ARF8 down-regulation compared to control, p-value=0.0045, fold change of 2.17. The results are indicative of total miR167 level in the transgenic plants.

### DESCRIPTION OF SPECIFIC EMBODIMENTS OF THE INVENTION

The present invention, in some embodiments thereof, relates to plants having improved abiotic stress tolerance and a method of improving abiotic stress tolerance of plants and plants generated thereby.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

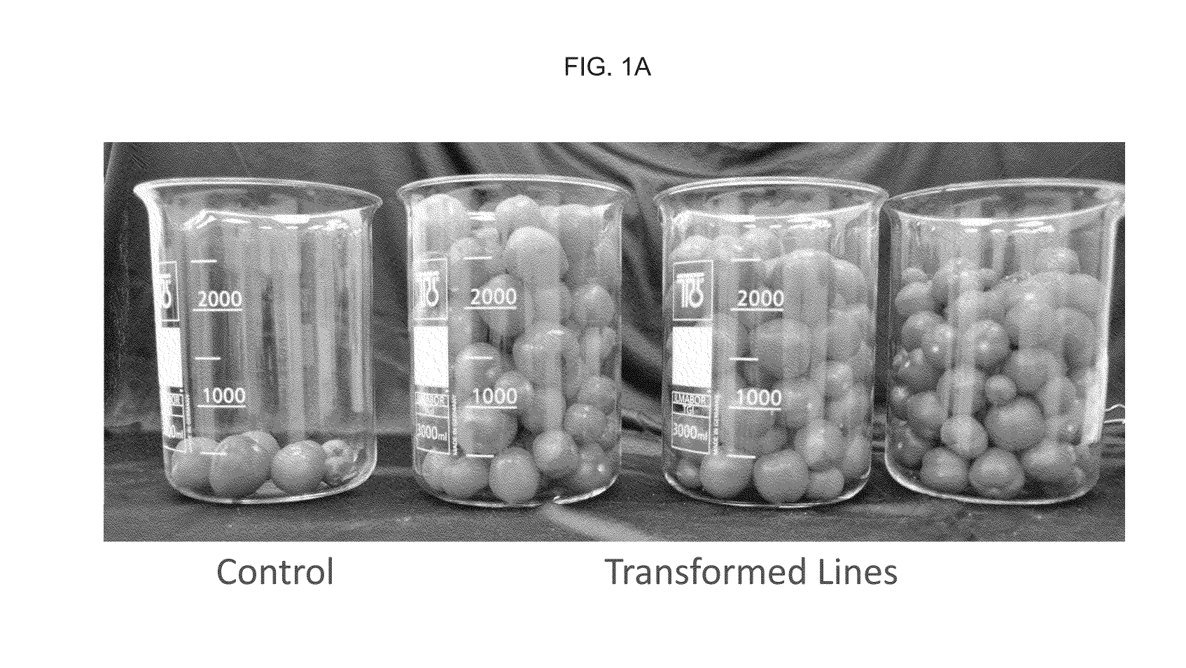

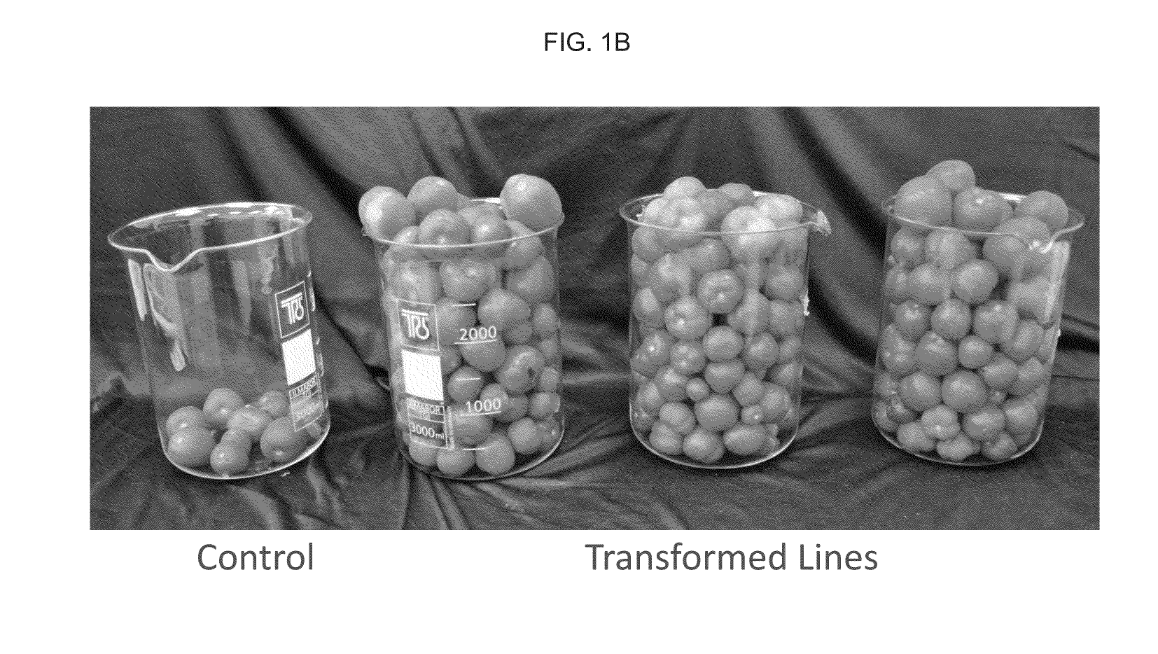

Whilst reducing the present invention to practice, the present inventors have identified novel selection criteria for miR167 expressing plants, which result in optimal resistance to abiotic stress and increased yield (see FIG. 1), while maintaining a normal plant phenotype.

Thus, according to an aspect of the invention, there is provided a method of improving abiotic stress tolerance of a plant. The method comprising genetically modifying the plant to express miRNA167 in an abiotic stress responsive manner, wherein a level of expression of total miR167 under the abiotic stress conditions is selected not exceeding 10 fold compared to same in the plant when grown under optimal conditions, thereby improving abiotic stress tolerance of the plant.

Examples of miR167 sequences which can be used along with the present teachings include, but are not limited to, those of Table 1 and the following homolog sequences (Table 2) as further described hereinbelow.

TABLE 1

| | | | Sequence for cloning into pORE- |
|---|---|---|---|
| | | | E2 using Bam HI (underlined) and |
| Mir | Mir | | KpnI (bold) restriction |
| Name | Sequence | Stem Loop Sequence/SEQ ID NO: | enzymes/SEQ ID NO: |
| ath- | TGAAGCTG | TGGTGCACCGGCATCTGATGAAGCTGCCAGC | GATCCTGAACAGAAAAATCTCTCTTTCTCTTT |
| miR1 | CCAGCATG | ATGATCTAATTAGCTTTCTTTATCCTTTGTT | CTTGATCTGCTACGGTGAAGTCTATGGTGCAC |
| 67a | ATCTA/1 | GTGTTTCATGACGATGGTTAAGAGATCAGTC | CGGCATCTGATGAAGCTGCCAGCATGATCTAA |
| | | TCGATTAGATCATGTTCGCAGTTTCACCCGT | TTAGCTTTCTTTATCCTTTGTTGTGTTTCATG |
| | | TGACTGTCGCACCC/2 | ACGATGGTTAAGAGATCAGTCTCGATTAGATC |
| | | | ATGTTCGCAGTTTCACCCGTTGACTGTCGCAC |
| | | | CCTTCTATAAACCCTAAATTTTCTCTCTATCT |
| | | | TTTTTAGTTTGATTTTCAAGACACTTTGTTTC |
| | | | TCAATCTTCAGTCTGATTTTGTGAGCTTACTT |
| | | | CTCTTTCTGAGGCTATAGGTAC/3 |

TABLE 2

| Homolog | Homolog Sequence SEQ ID NO:/ | Homolog |
|---|---|---|
| Name | hairpin SEQ ID NO: | length |
| ahy-miR167- | TGAAGCTGCCAGCATGATCTT/4/370 | 21 |
| 5p | | |
| aly-miR167a | TGAAGCTGCCAGCATGATCTA/5/371 | 21 |
| aly-miR167b | TGAAGCTGCCAGCATGATCTA/6/372 | 21 |
| aly-miR167c | TAAGCTGCCAGCATGATCTTG/7/373 | 21 |
| aly-miR167d | TGAAGCTGCCAGCATGATCTGG/8/374 | 22 |
| aqc-miR167 | TCAAGCTGCCAGCATGATCTA/9/375 | 21 |
| ath-miR167b | TGAAGCTGCCAGCATGATCTA/10/376 | 21 |
| ath-miR167c | TAAGCTGCCAGCATGATCTTG/11/377 | 21 |
| ath-miR167d | TGAAGCTGCCAGCATGATCTGG/12/378 | 22 |
| ath-miR167m | TGAAGCTGCCAGCATGATCTG/13/379 | 21 |
| bdi-miR167 | TGAAGCTGCCAGCATGATCTA/14/380 | 21 |
| bdi-miR167a | TGAAGCTGCCAGCATGATCTA/15/381 | 21 |
| bdi-miR167b | TGAAGCTGCCAGCATGATCTA/16/382 | 21 |
| bdi-miR167c | TGAAGCTGCCAGCATGATCTGA/17/383 | 22 |
| bdi-miR167d | TGAAGCTGCCAGCATGATCTGA/18/384 | 22 |
| bna-miR167a | TGAAGCTGCCAGCATGATCTAA/19/385 | 22 |
| bna-miR167b | TGAAGCTGCCAGCATGATCTAA/20/386 | 22 |
| bna-miR167c | TGAAGCTGCCAGCATGATCTA/21/387 | 21 |
| bra-miR167a | TGAAGCTGCCAGCATGATCTA/22/388 | 21 |
| bra-miR167b | TGAAGCTGCCAGCATGATCTA/23/389 | 21 |
| bra-miR167c | TGAAGCTGCCAGCATGATCTA/24/390 | 21 |
| bra-miR167d | TGAAGCTGCCAGCATGATCTA/25/391 | 21 |
| ccl-miR167a | TGAAGCTGCCAGCATGATCTGA/26/392 | 22 |
| ccl-miR167b | TGAAGCTGCCAGCATGATCTGA/27/393 | 22 |
| cle-miR167 | TGAAGCTGCCAGCATGATCTG/28/394 | 21 |
| csi-miR167a | TGAAGCTGCCAGCATGATCTG/29/395 | 21 |
| csi-miR167b | TGAAGCTGCCAGCATGATCTT/30/396 | 21 |
| csi-miR167c | TGAAGCTGCCAGCATGATCTG/31/397 | 21 |
| ctr-miR167 | TGAAGCTGCCAGCATGATCTGA/32/398 | 22 |
| ghr-miR167 | TGAAGCTGCCAGCATGATCTA/33/399 | 21 |
| gma-miR167a | TGAAGCTGCCAGCATGATCTA/34/400 | 21 |
| gma-miR167b | TGAAGCTGCCAGCATGATCTA/35/401 | 21 |
| gma-miR167c | TGAAGCTGCCAGCATGATCTG/36/402 | 21 |
| gma-miR167d | TGAAGCTGCCAGCATGATCTA/37/403 | 21 |
| gma-miR167e | TGAAGCTGCCAGCATGATCTT/38/404 | 21 |
| gma-miR167f | TGAAGCTGCCAGCATGATCTT/39/405 | 21 |
| gma-miR167g | TGAAGCTGCCAGCATGATCTGA/40/406 | 22 |
| gma-miR167h | ATCATGCTGGCAGCTTCAACTGGT/41/407 | 24 |
| gma-miR167i | TCATGCTGGCAGCTTCAACTGGT/42/408 | 23 |
| gma-miR167j | TGAAGCTGCCAGCATGATCTG/43/409 | 21 |
| gma-miR167n | TGAAGCTGCCAGCATGATCT/44/410 | 20 |
| gma-miR167o | TGAAGCTGCCAGCATGATCTG/45/411 | 21 |
| gso-miR167a | TGAAGCTGCCAGCATGATCTG/46/412 | 21 |
| ini-miR167 | TGAAGCTGCCAGCATGATCTG/47/413 | 21 |
| lja-miR167 | TGAAGCTGCCAGCATGATCTG/48/414 | 21 |
| mtr-miR167 | TGAAGCTGCCAGCATGATCTA/49/415 | 21 |
| mtr-miR167b | TGAAGCTGCCAGCATGATCTG/50/416 | 21 |
| osa-miR167a | TGAAGCTGCCAGCATGATCTA/51/417 | 21 |
| osa- | ATCATGCATGACAGCCTCATTT/52/418 | 22 |
| miR167a* | | |
| osa-miR167b | TGAAGCTGCCAGCATGATCTA/53/419 | 21 |
| osa-miR167c | TGAAGCTGCCAGCATGATCTA/54/420 | 21 |
| osa-miR167d | TGAAGCTGCCAGCATGATCTG/55/421 | 21 |
| osa-miR167e | TGAAGCTGCCAGCATGATCTG/56/422 | 21 |
| osa-miR167f | TGAAGCTGCCAGCATGATCTG/57/423 | 21 |
| osa-miR167g | TGAAGCTGCCAGCATGATCTG/58/424 | 21 |
| osa-miR167h | TGAAGCTGCCAGCATGATCTG/59/425 | 21 |
| osa-miR167i | TGAAGCTGCCAGCATGATCTG/60/426 | 21 |
| osa-miR167j | TGAAGCTGCCAGCATGATCTG/61/427 | 21 |
| osa-miR167m | TGAAGCTGCCAGCATGATCTG/62/428 | 21 |
| osa-miR167n | TGAAGCTGCCAGCATGATCTG/63/429 | 21 |
| pco-miR167 | TGAAGCTGCCAGCATGATCTT/64/430 | 21 |
| ppl-miR167a | TGAAGCTGCCAGCATGATCTA/65/431 | 21 |
| ppl-miR167b | TGAAGCTGCCAGCATGATCTG/66/432 | 21 |
| ppt-miR167 | GGAAGCTGCCAGCATGATCCT/67/433 | 21 |
| ptc-miR167a | TGAAGCTGCCAGCATGATCTA/68/434 | 21 |
| ptc-miR167b | TGAAGCTGCCAGCATGATCTA/69/435 | 21 |
| ptc-miR167c | TGAAGCTGCCAGCATGATCTA/70/436 | 21 |
| ptc-miR167d | TGAAGCTGCCAGCATGATCTA/71/437 | 21 |
| ptc-miR167e | TGAAGCTGCCAGCATGATCTG/72/438 | 21 |
| ptc-miR167f | TGAAGCTGCCAGCATGATCTT/73/439 | 21 |
| ptc-miR167g | TGAAGCTGCCAGCATGATCTT/74/440 | 21 |
| ptc-miR167h | TGAAGCTGCCAACATGATCTG/75/441 | 21 |
| pts-miR167 | TGAAGCTGCCAGCATGATCTG/76/442 | 21 |
| rco-miR167a | TGAAGCTGCCAGCATGATCTA/77/443 | 21 |
| rco-miR167b | TGAAGCTGCCAGCATGATCTA/78/444 | 21 |
| rco-miR167c | TGAAGCTGCCAGCATGATCTGG/79/445 | 22 |
| sbi-miR167a | TGAAGCTGCCAGCATGATCTA/80/446 | 21 |
| sbi-miR167b | TGAAGCTGCCAGCATGATCTA/81/447 | 21 |
| sbi-miR167c | TGAAGCTGCCAGCATGATCTG/82/448 | 21 |
| sbi-miR167d | TGAAGCTGCCAGCATGATCTG/83/449 | 21 |
| sbi-miR167e | TGAAGCTGCCAGCATGATCTG/84/450 | 21 |
| sbi-miR167f | TGAAGCTGCCAGCATGATCTG/85/451 | 21 |
| sbi-miR167g | TGAAGCTGCCAGCATGATCTG/86/452 | 21 |
| sbi-miR167h | TGAAGCTGCCAGCATGATCTG/87/453 | 21 |
| sbi-miR167i | TGAAGCTGCCAGCATGATCTA/88/454 | 21 |
| sly-miR167 | TGAAGCTGCCAGCATGATCTA/89/455 | 21 |
| sof-miR167a | TGAAGCTGCCAGCATGATCTG/90/456 | 21 |
| sof-miR167b | TGAAGCTGCCAGCATGATCTG/91/457 | 21 |
| ssp-miR167 | TGAAGCTGCCAGCATGATCTG/92/458 | 21 |
| ssp-miR167b | TGAAGCTGCCAGCATGATCTG/93/459 | 21 |
| tae-miR167 | TGAAGCTGCCAGCATGATCTA/94/460 | 21 |
| tae-miR167b | TGAAGCTGACAGCATGATCTA/95/461 | 21 |
| tcc-miR167a | TGAAGCTGCCAGCATGATCTA/96/462 | 21 |
| tcc-miR167b | TGAAGCTGCCAGCATGATCTA/97/463 | 21 |
| tcc-miR167c | TGAAGCTGCCAGCATGATCTT/98/464 | 21 |
| vvi-miR167a | TGAAGCTGCCAGCATGATCTG/99/465 | 21 |
| vvi-miR167b | TGAAGCTGCCAGCATGATCTA/100/466 | 21 |
| vvi-miR167c | TGAAGCTGCCAGCATGATCTC/101/467 | 21 |
| vvi-miR167d | TGAAGCTGCCAGCATGATCTA/102/468 | 21 |
| vvi-miR167e | TGAAGCTGCCAGCATGATCTA/103/469 | 21 |
| zma-miR167a | TGAAGCTGCCAGCATGATCTA/104/470 | 21 |
| zma- | GATCATGCATGACAGCCTCATT/105/471 | 22 |
| miR167a* | | |
| zma-miR167b | TGAAGCTGCCAGCATGATCTA/106/472 | 21 |
| zma-miR167c | TGAAGCTGCCAGCATGATCTA/107/473 | 21 |
| zma-miR167d | TGAAGCTGCCAGCATGATCTA/108/474 | 21 |
| zma- | GGTCATGCTGCTGCAGCCTCACT/109/475 | 23 |
| miR167d* | | |
| zma-miR167e | TGAAGCTGCCAGCATGATCTG/110/476 | 21 |
| zma- | GATCATGCTGTGCAGTTTCATC/111/477 | 22 |
| miR167e* | | |
| zma-miR167f | TGAAGCTGCCAGCATGATCTG/112/478 | 21 |
| zma-miR167g | TGAAGCTGCCAGCATGATCTG/113/479 | 21 |
| zma-miR167h | TGAAGCTGCCAGCATGATCTG/114/480 | 21 |
| zma-miR167i | TGAAGCTGCCAGCATGATCTG/115/481 | 21 |
| zma-miR167j | TGAAGCTGCCAGCATGATCTG/116/482 | 21 |
| zma-miR167k | TGAAGCTGCCAGCATGATCTG/117/483 | 21 |
| zma-miR167l | TGAAGCTGCCAGCATGATCTG/118/484 | 21 |
| zma-miR167m | TGAAGCTGCCAGCATGATCTG/119/485 | 21 |
| zma-miR167n | TGAAGCTGCCAGCATGATCTA/120/486 | 21 |
| zma-miR167o | TGAAGCTGCCAGCATGATCTA/121/487 | 21 |
| zma-miR167p | TGAAGCTGCCAGCATGATCTA/122/488 | 21 |
| zma-miR167q | TGAAGCTGCCAGCATGATCTA/123/489 | 21 |
| zma-miR167r | TGAAGCTGCCAGCATGATCTA/124/490 | 21 |
| zma-miR167s | TGAAGCTGCCAGCATGATCTA/125/491 | 21 |
| zma-miR167t | TGAAGCTGCCAGCATGATCTA/126/492 | 21 |
| zma-miR167u | TGAAGCTGCCACATGATCTG/127/493 | 20 |
| ahy-miR167- | TGAAGCTGCCAGCATGATCTT/128/494 | 21 |
| 5p | | |
| aly-miR167a | TGAAGCTGCCAGCATGATCTA/129/495 | 21 |
| aly-miR167b | TGAAGCTGCCAGCATGATCTA/130/496 | 21 |
| aly-miR167c | TAAGCTGCCAGCATGATCTTG/131/497 | 21 |
| aly-miR167d | TGAAGCTGCCAGCATGATCTGG/132/498 | 22 |
| aqc-miR167 | TCAAGCTGCCAGCATGATCTA/133/499 | 21 |
| ath-miR167a | TGAAGCTGCCAGCATGATCTA/134/500 | 21 |
| ath-miR167b | TGAAGCTGCCAGCATGATCTA/135/501 | 21 |
| ath-miR167d | TGAAGCTGCCAGCATGATCTGG/136/502 | 22 |
| ath-miR167m | TGAAGCTGCCAGCATGATCTG/137/503 | 21 |
| bdi-miR167 | TGAAGCTGCCAGCATGATCTA/138/504 | 21 |
| bdi-miR167a | TGAAGCTGCCAGCATGATCTA/139/505 | 21 |
| bdi-miR167b | TGAAGCTGCCAGCATGATCTA/140/506 | 21 |
| bdi-miR167c | TGAAGCTGCCAGCATGATCTGA/141/507 | 22 |
| bdi-miR167d | TGAAGCTGCCAGCATGATCTGA/142/508 | 22 |
| bna-miR167a | TGAAGCTGCCAGCATGATCTAA/143/509 | 22 |
| bna-miR167b | TGAAGCTGCCAGCATGATCTAA/144/510 | 22 |
| bna-miR167c | TGAAGCTGCCAGCATGATCTA/145/511 | 21 |
| bra-miR167a | TGAAGCTGCCAGCATGATCTA/146/512 | 21 |
| bra-miR167b | TGAAGCTGCCAGCATGATCTA/147/513 | 21 |
| bra-miR167c | TGAAGCTGCCAGCATGATCTA/148/514 | 21 |
| bra-miR167d | TGAAGCTGCCAGCATGATCTA/149/515 | 21 |
| ccl-miR167a | TGAAGCTGCCAGCATGATCTGA/150/516 | 22 |
| ccl-miR167b | TGAAGCTGCCAGCATGATCTGA/151/517 | 22 |
| cle-miR167 | TGAAGCTGCCAGCATGATCTG/152/518 | 21 |
| csi-miR167a | TGAAGCTGCCAGCATGATCTG/153/519 | 21 |
| csi-miR167b | TGAAGCTGCCAGCATGATCTT/154/520 | 21 |
| csi-miR167c | TGAAGCTGCCAGCATGATCTG/155/521 | 21 |
| ctr-miR167 | TGAAGCTGCCAGCATGATCTGA/156/522 | 22 |
| ghr-miR167 | TGAAGCTGCCAGCATGATCTA/157/523 | 21 |
| gma-miR167a | TGAAGCTGCCAGCATGATCTA/158/524 | 21 |
| gma-miR167b | TGAAGCTGCCAGCATGATCTA/159/525 | 21 |
| gma-miR167c | TGAAGCTGCCAGCATGATCTG/160/526 | 21 |
| gma-miR167d | TGAAGCTGCCAGCATGATCTA/161/527 | 21 |
| gma-miR167e | TGAAGCTGCCAGCATGATCTT/162/528 | 21 |
| gma-miR167f | TGAAGCTGCCAGCATGATCTT/163/529 | 21 |
| gma-miR167g | TGAAGCTGCCAGCATGATCTGA/164/530 | 22 |
| gma-miR167h | ATCATGCTGGCAGCTTCAACTGGT/165/ | 24 |
| | 531 | |
| gma-miR167i | TCATGCTGGCAGCTTCAACTGGT/166/532 | 23 |
| gma-miR167j | TGAAGCTGCCAGCATGATCTG/167/533 | 21 |
| gma-miR167n | TGAAGCTGCCAGCATGATCT/168/534 | 20 |
| gma-miR167o | TGAAGCTGCCAGCATGATCTG/169/535 | 21 |
| gso-miR167a | TGAAGCTGCCAGCATGATCTG/170/536 | 21 |
| ini-miR167 | TGAAGCTGCCAGCATGATCTG/171/537 | 21 |
| lja-miR167 | TGAAGCTGCCAGCATGATCTG/172/538 | 21 |
| mtr-miR167 | TGAAGCTGCCAGCATGATCTA/173/539 | 21 |
| mtr-miR167b | TGAAGCTGCCAGCATGATCTG/174/540 | 21 |
| osa-miR167a | TGAAGCTGCCAGCATGATCTA/175/541 | 21 |
| osa- | ATCATGCATGACAGCCTCATTT/176/542 | 22 |
| miR167a* | | |
| osa-miR167b | TGAAGCTGCCAGCATGATCTA/177/543 | 21 |
| osa-miR167c | TGAAGCTGCCAGCATGATCTA/178/544 | 21 |
| osa-miR167d | TGAAGCTGCCAGCATGATCTG/179/545 | 21 |
| osa-miR167e | TGAAGCTGCCAGCATGATCTG/180/546 | 21 |
| osa-miR167f | TGAAGCTGCCAGCATGATCTG/181/547 | 21 |
| osa-miR167g | TGAAGCTGCCAGCATGATCTG/182/548 | 21 |
| osa-miR167h | TGAAGCTGCCAGCATGATCTG/183/549 | 21 |
| osa-miR167i | TGAAGCTGCCAGCATGATCTG/184/550 | 21 |
| osa-miR167j | TGAAGCTGCCAGCATGATCTG/185/551 | 21 |
| osa-miR167m | TGAAGCTGCCAGCATGATCTG/186/552 | 21 |
| osa-miR167n | TGAAGCTGCCAGCATGATCTG/187/553 | 21 |
| pco-miR167 | TGAAGCTGCCAGCATGATCTT/188/554 | 21 |
| ppl-miR167a | TGAAGCTGCCAGCATGATCTA/189/555 | 21 |
| ppl-miR167b | TGAAGCTGCCAGCATGATCTG/190/556 | 21 |
| ppt-miR167 | GGAAGCTGCCAGCATGATCCT/191/557 | 21 |
| ptc-miR167a | TGAAGCTGCCAGCATGATCTA/192/558 | 21 |
| ptc-miR167b | TGAAGCTGCCAGCATGATCTA/193/559 | 21 |
| ptc-miR167c | TGAAGCTGCCAGCATGATCTA/194/560 | 21 |
| ptc-miR167d | TGAAGCTGCCAGCATGATCTA/195/561 | 21 |
| ptc-miR167e | TGAAGCTGCCAGCATGATCTG/196/562 | 21 |
| ptc-miR167f | TGAAGCTGCCAGCATGATCTT/197/563 | 21 |
| ptc-miR167g | TGAAGCTGCCAGCATGATCTT/198/564 | 21 |
| ptc-miR167h | TGAAGCTGCCAACATGATCTG/199/565 | 21 |
| pts-miR167 | TGAAGCTGCCAGCATGATCTG/200/566 | 21 |
| rco-miR167a | TGAAGCTGCCAGCATGATCTA/201/567 | 21 |
| rco-miR167b | TGAAGCTGCCAGCATGATCTA/202/568 | 21 |
| rco-miR167c | TGAAGCTGCCAGCATGATCTGG/203/569 | 22 |
| sbi-miR167a | TGAAGCTGCCAGCATGATCTA/204/570 | 21 |
| sbi-miR167b | TGAAGCTGCCAGCATGATCTA/205/571 | 21 |
| sbi-miR167c | TGAAGCTGCCAGCATGATCTG/206/572 | 21 |
| sbi-miR167d | TGAAGCTGCCAGCATGATCTG/207/573 | 21 |
| sbi-miR167e | TGAAGCTGCCAGCATGATCTG/208/574 | 21 |
| sbi-miR167f | TGAAGCTGCCAGCATGATCTG/209/575 | 21 |
| sbi-miR167g | TGAAGCTGCCAGCATGATCTG/210/576 | 21 |
| sbi-miR167h | TGAAGCTGCCAGCATGATCTG/211/577 | 21 |
| sbi-miR167i | TGAAGCTGCCAGCATGATCTA/212/578 | 21 |
| sly-miR167 | TGAAGCTGCCAGCATGATCTA/213/579 | 21 |
| sof-miR167a | TGAAGCTGCCAGCATGATCTG/214/580 | 21 |
| sof-miR167b | TGAAGCTGCCAGCATGATCTG/215/581 | 21 |
| ssp-miR167 | TGAAGCTGCCAGCATGATCTG/216/582 | 21 |
| ssp-miR167b | TGAAGCTGCCAGCATGATCTG/217/583 | 21 |
| tae-miR167 | TGAAGCTGCCAGCATGATCTA/218/584 | 21 |
| tae-miR167b | TGAAGCTGACAGCATGATCTA/219/585 | 21 |
| tcc-miR167a | TGAAGCTGCCAGCATGATCTA/220/586 | 21 |
| tcc-miR167b | TGAAGCTGCCAGCATGATCTA/221/587 | 21 |
| tcc-miR167c | TGAAGCTGCCAGCATGATCTT/222/588 | 21 |
| vvi-miR167a | TGAAGCTGCCAGCATGATCTG/223/589 | 21 |
| vvi-miR167b | TGAAGCTGCCAGCATGATCTA/224/590 | 21 |
| vvi-miR167c | TGAAGCTGCCAGCATGATCTC/225/591 | 21 |
| vvi-miR167d | TGAAGCTGCCAGCATGATCTA/226/592 | 21 |
| vvi-miR167e | TGAAGCTGCCAGCATGATCTA/227/593 | 21 |
| zma-miR167a | TGAAGCTGCCAGCATGATCTA/228/594 | 21 |
| zma-miR167b | TGAAGCTGCCAGCATGATCTA/229/595 | 21 |
| zma-miR167c | TGAAGCTGCCAGCATGATCTA/230/596 | 21 |
| zma-miR167d | TGAAGCTGCCAGCATGATCTA/231/597 | 21 |
| zma-miR167e | TGAAGCTGCCAGCATGATCTG/232/598 | 21 |
| zma-miR167f | TGAAGCTGCCAGCATGATCTG/233/599 | 21 |
| zma-miR167g | TGAAGCTGCCAGCATGATCTG/234/600 | 21 |
| zma-miR167h | TGAAGCTGCCAGCATGATCTG/235/601 | 21 |
| zma-miR167i | TGAAGCTGCCAGCATGATCTG/236/602 | 21 |
| zma-miR167j | TGAAGCTGCCAGCATGATCTG/237/603 | 21 |
| zma-miR167k | TGAAGCTGCCAGCATGATCTG/238/604 | 21 |
| zma-miR167l | TGAAGCTGCCAGCATGATCTG/239/605 | 21 |
| zma-miR167m | TGAAGCTGCCAGCATGATCTG/240/606 | 21 |
| zma-miR167n | TGAAGCTGCCAGCATGATCTA/241/607 | 21 |
| zma-miR167o | TGAAGCTGCCAGCATGATCTA/242/608 | 21 |
| zma-miR167p | TGAAGCTGCCAGCATGATCTA/243/609 | 21 |
| zma-miR167q | TGAAGCTGCCAGCATGATCTA/244/610 | 21 |
| zma-miR167r | TGAAGCTGCCAGCATGATCTA/245/611 | 21 |
| zma-miR167s | TGAAGCTGCCAGCATGATCTA/246/612 | 21 |
| zma-miR167t | TGAAGCTGCCAGCATGATCTA/247/613 | 21 |
| zma-miR167u | TGAAGCTGCCACATGATCTG/248/614 | 20 |
| ahy-miR167- | TGAAGCTGCCAGCATGATCTT/249/615 | 21 |
| 5p | | |
| aly-miR167a | TGAAGCTGCCAGCATGATCTA/250/616 | 21 |
| aly-miR167b | TGAAGCTGCCAGCATGATCTA/251/617 | 21 |
| aly-miR167c | TAAGCTGCCAGCATGATCTTG/252/618 | 21 |
| aly-miR167d | TGAAGCTGCCAGCATGATCTGG/253/619 | 22 |
| aqc-miR167 | TCAAGCTGCCAGCATGATCTA/254/620 | 21 |
| ath-miR167a | TGAAGCTGCCAGCATGATCTA/255/621 | 21 |
| ath-miR167b | TGAAGCTGCCAGCATGATCTA/256/622 | 21 |
| ath-miR167c | TAAGCTGCCAGCATGATCTTG/257/623 | 21 |
| ath-miR167m | TGAAGCTGCCAGCATGATCTG/258/624 | 21 |
| bdi-miR167 | TGAAGCTGCCAGCATGATCTA/259/625 | 21 |
| bdi-miR167a | TGAAGCTGCCAGCATGATCTA/260/626 | 21 |
| bdi-miR167b | TGAAGCTGCCAGCATGATCTA/261/627 | 21 |
| bdi-miR167c | TGAAGCTGCCAGCATGATCTGA/262/628 | 22 |
| bdi-miR167d | TGAAGCTGCCAGCATGATCTGA/263/629 | 22 |
| bna-miR167a | TGAAGCTGCCAGCATGATCTAA/264/630 | 22 |
| bna-miR167b | TGAAGCTGCCAGCATGATCTAA/265/631 | 22 |
| bna-miR167c | TGAAGCTGCCAGCATGATCTA/266/632 | 21 |
| bra-miR167a | TGAAGCTGCCAGCATGATCTA/267/633 | 21 |
| bra-miR167b | TGAAGCTGCCAGCATGATCTA/268/634 | 21 |
| bra-miR167c | TGAAGCTGCCAGCATGATCTA/269/635 | 21 |
| bra-miR167d | TGAAGCTGCCAGCATGATCTA/270/636 | 21 |
| ccl-miR167a | TGAAGCTGCCAGCATGATCTGA/271/637 | 22 |
| ccl-miR167b | TGAAGCTGCCAGCATGATCTGA/272/638 | 22 |
| cle-miR167 | TGAAGCTGCCAGCATGATCTG/273/639 | 21 |
| csi-miR167a | TGAAGCTGCCAGCATGATCTG/274/640 | 21 |
| csi-miR167b | TGAAGCTGCCAGCATGATCTT/275/641 | 21 |
| csi-miR167c | TGAAGCTGCCAGCATGATCTG/276/642 | 21 |
| ctr-miR167 | TGAAGCTGCCAGCATGATCTGA/277/643 | 22 |
| ghr-miR167 | TGAAGCTGCCAGCATGATCTA/278/644 | 21 |
| gma-miR167a | TGAAGCTGCCAGCATGATCTA/279/645 | 21 |
| gma-miR167b | TGAAGCTGCCAGCATGATCTA/280/646 | 21 |
| gma-miR167c | TGAAGCTGCCAGCATGATCTG/281/647 | 21 |
| gma-miR167d | TGAAGCTGCCAGCATGATCTA/282/648 | 21 |
| gma-miR167e | TGAAGCTGCCAGCATGATCTT/283/649 | 21 |
| gma-miR167f | TGAAGCTGCCAGCATGATCTT/284/650 | 21 |
| gma-miR167g | TGAAGCTGCCAGCATGATCTGA/285/651 | 22 |
| gma-miR167h | ATCATGCTGGCAGCTTCAACTGGT/286/ | 24 |
| | 652 | |
| gma-miR167i | TCATGCTGGCAGCTTCAACTGGT/287/653 | 23 |
| gma-miR167j | TGAAGCTGCCAGCATGATCTG/288/654 | 21 |
| gma-miR167n | TGAAGCTGCCAGCATGATCT/289/655 | 20 |
| gma-miR167o | TGAAGCTGCCAGCATGATCTG/290/656 | 21 |
| gso-miR167a | TGAAGCTGCCAGCATGATCTG/291/657 | 21 |
| ini-miR167 | TGAAGCTGCCAGCATGATCTG/292/658 | 21 |
| lja-miR167 | TGAAGCTGCCAGCATGATCTG/293/659 | 21 |
| mtr-miR167 | TGAAGCTGCCAGCATGATCTA/294/660 | 21 |
| mtr-miR167b | TGAAGCTGCCAGCATGATCTG/295/661 | 21 |
| osa-miR167a | TGAAGCTGCCAGCATGATCTA/296/662 | 21 |
| osa- | ATCATGCATGACAGCCTCATTT/297/663 | 22 |
| miR167a* | | |
| osa-miR167b | TGAAGCTGCCAGCATGATCTA/298/664 | 21 |
| osa-miR167c | TGAAGCTGCCAGCATGATCTA/299/665 | 21 |
| osa-miR167d | TGAAGCTGCCAGCATGATCTG/300/666 | 21 |
| osa-miR167e | TGAAGCTGCCAGCATGATCTG/301/667 | 21 |
| osa-miR167f | TGAAGCTGCCAGCATGATCTG/302/668 | 21 |
| osa-miR167g | TGAAGCTGCCAGCATGATCTG/303/669 | 21 |
| osa-miR167h | TGAAGCTGCCAGCATGATCTG/304/670 | 21 |
| osa-miR167i | TGAAGCTGCCAGCATGATCTG/305/671 | 21 |
| osa-miR167j | TGAAGCTGCCAGCATGATCTG/306/672 | 21 |
| osa-miR167m | TGAAGCTGCCAGCATGATCTG/307/673 | 21 |
| osa-miR167n | TGAAGCTGCCAGCATGATCTG/308/674 | 21 |
| pco-miR167 | TGAAGCTGCCAGCATGATCTT/309/675 | 21 |
| ppl-miR167a | TGAAGCTGCCAGCATGATCTA/310/676 | 21 |
| ppl-miR167b | TGAAGCTGCCAGCATGATCTG/311/677 | 21 |
| ppt-miR167 | GGAAGCTGCCAGCATGATCCT/312/678 | 21 |
| ptc-miR167a | TGAAGCTGCCAGCATGATCTA/313/679 | 21 |
| ptc-miR167b | TGAAGCTGCCAGCATGATCTA/314/680 | 21 |
| ptc-miR167c | TGAAGCTGCCAGCATGATCTA/315/681 | 21 |
| ptc-miR167d | TGAAGCTGCCAGCATGATCTA/316/682 | 21 |
| ptc-miR167e | TGAAGCTGCCAGCATGATCTG/317/683 | 21 |
| ptc-miR167f | TGAAGCTGCCAGCATGATCTT/318/684 | 21 |
| ptc-miR167g | TGAAGCTGCCAGCATGATCTT/319/685 | 21 |
| ptc-miR167h | TGAAGCTGCCAACATGATCTG/320/686 | 21 |
| pts-miR167 | TGAAGCTGCCAGCATGATCTG/321/687 | 21 |
| rco-miR167a | TGAAGCTGCCAGCATGATCTA/322/688 | 21 |
| rco-miR167b | TGAAGCTGCCAGCATGATCTA/323/689 | 21 |
| rco-miR167c | TGAAGCTGCCAGCATGATCTGG/324/690 | 22 |
| sbi-miR167a | TGAAGCTGCCAGCATGATCTA/325/691 | 21 |
| sbi-miR167b | TGAAGCTGCCAGCATGATCTA/326/692 | 21 |
| sbi-miR167c | TGAAGCTGCCAGCATGATCTG/327/693 | 21 |
| sbi-miR167d | TGAAGCTGCCAGCATGATCTG/328/694 | 21 |
| sbi-miR167e | TGAAGCTGCCAGCATGATCTG/329/695 | 21 |
| sbi-miR167f | TGAAGCTGCCAGCATGATCTG/330/696 | 21 |
| sbi-miR167g | TGAAGCTGCCAGCATGATCTG/331/697 | 21 |
| sbi-miR167h | TGAAGCTGCCAGCATGATCTG/332/698 | 21 |
| sbi-miR167i | TGAAGCTGCCAGCATGATCTA/333/699 | 21 |
| sly-miR167 | TGAAGCTGCCAGCATGATCTA/334/700 | 21 |
| sof-miR167a | TGAAGCTGCCAGCATGATCTG/335/701 | 21 |
| sof-miR167b | TGAAGCTGCCAGCATGATCTG/336/702 | 21 |
| ssp-miR167 | TGAAGCTGCCAGCATGATCTG/337/703 | 21 |
| ssp-miR167b | TGAAGCTGCCAGCATGATCTG/338/704 | 21 |
| tae-miR167 | TGAAGCTGCCAGCATGATCTA/339/705 | 21 |
| tae-miR167b | TGAAGCTGACAGCATGATCTA/340/706 | 21 |
| tcc-miR167a | TGAAGCTGCCAGCATGATCTA/341/707 | 21 |
| tcc-miR167b | TGAAGCTGCCAGCATGATCTA/342/708 | 21 |
| tcc-miR167c | TGAAGCTGCCAGCATGATCTT/343/709 | 21 |
| vvi-miR167a | TGAAGCTGCCAGCATGATCTG/344/710 | 21 |
| vvi-miR167b | TGAAGCTGCCAGCATGATCTA/345/711 | 21 |
| vvi-miR167c | TGAAGCTGCCAGCATGATCTC/346/712 | 21 |
| vvi-miR167d | TGAAGCTGCCAGCATGATCTA/347/713 | 21 |
| vvi-miR167e | TGAAGCTGCCAGCATGATCTA/348/714 | 21 |
| zma-miR167a | TGAAGCTGCCAGCATGATCTA/349/715 | 21 |
| zma-miR167b | TGAAGCTGCCAGCATGATCTA/350/716 | 21 |
| zma-miR167c | TGAAGCTGCCAGCATGATCTA/351/717 | 21 |
| zma-miR167d | TGAAGCTGCCAGCATGATCTA/352/718 | 21 |
| zma-miR167e | TGAAGCTGCCAGCATGATCTG/353/719 | 21 |
| zma-miR167f | TGAAGCTGCCAGCATGATCTG/354/720 | 21 |
| zma-miR167g | TGAAGCTGCCAGCATGATCTG/355/721 | 21 |
| zma-miR167h | TGAAGCTGCCAGCATGATCTG/356/722 | 21 |
| zma-miR167i | TGAAGCTGCCAGCATGATCTG/357/723 | 21 |
| zma-miR167j | TGAAGCTGCCAGCATGATCTG/358/724 | 21 |
| zma-miR167k | TGAAGCTGCCAGCATGATCTG/359/725 | 21 |
| zma-miR167l | TGAAGCTGCCAGCATGATCTG/360/726 | 21 |
| zma-miR167m | TGAAGCTGCCAGCATGATCTG/361/727 | 21 |
| zma-miR167n | TGAAGCTGCCAGCATGATCTA/362/728 | 21 |
| zma-miR167o | TGAAGCTGCCAGCATGATCTA/363/729 | 21 |
| zma-miR167p | TGAAGCTGCCAGCATGATCTA/364/730 | 21 |
| zma-miR167q | TGAAGCTGCCAGCATGATCTA/365/731 | 21 |
| zma-miR167r | TGAAGCTGCCAGCATGATCTA/366/732 | 21 |
| zma-miR167s | TGAAGCTGCCAGCATGATCTA/367/733 | 21 |
| zma-miR167t | TGAAGCTGCCAGCATGATCTA/368/734 | 21 |
| zma-miR167u | TGAAGCTGCCACATGATCTG/369/735 | 20 |

The term “plant” as used herein encompasses whole plants, ancestors and progeny of the plants, grafted plantsand plant parts, including seeds, shoots, stems, roots (including tubers), rootstock, scion, and isolated plant cells, tissues and organs. The plant may be in any form including suspension cultures, embryos, meristematic regions, callus tissue, leaves, gametophytes, sporophytes, pollen, and microspores.

As used herein the phrase “plant cell” refers to plant cells which are derived and isolated from disintegrated plant cell tissue or plant cell cultures.

As used herein the phrase “plant cell culture” refers to any type of native (naturally occurring) plant cells, plant cell lines and genetically modified plant cells, which are not assembled to form a complete plant, such that at least one biological structure of a plant is not present. Optionally, the plant cell culture of this aspect of the present invention may comprise a particular type of a plant cell or a plurality of different types of plant cells. It should be noted that optionally plant cultures featuring a particular type of plant cell may be originally derived from a plurality of different types of such plant cells.

Any commercially or scientifically valuable plant is envisaged in accordance with some embodiments of the invention. Plants that are particularly useful in the methods of the invention include all plants which belong to the super family Viridiplantae, in particular monocotyledonous and dicotyledonous plants including a fodder or forage legume, ornamental plant, food crop, tree, or shrub selected from the list comprising Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Canna indica, Capsicum spp., Cassia spp., Centroema pubescens, Chacoomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Dibeteropogon amplectens, Dioclea spp, Dolichos spp., Dorycnium rectum, Echinochloa pyramidalis, Ehraffia spp., Eleusine coracana, Eragrestis spp., Erythrina spp., Eucalyptus spp., Euclea schimperi, Eulalia vi/losa, Pagopyrum spp., Feijoa sellowlana, Fragaria spp., Flemingia spp, Freycinetia banksli, Geranium thunbergii, GinAgo biloba, Glycine javanica, Gliricidia spp, Gossypium hirsutum, Grevillea spp., Guibourtia coleosperma, Hedysarum spp., Hemaffhia altissima, Heteropogon contoffus, Hordeum vulgare, Hyparrhenia rufa, Hypericum erectum, Hypeffhelia dissolute, Indigo incamata, Iris spp., Leptarrhena pyrolifolia, Lespediza spp., Lettuca spp., Leucaena leucocephala, Loudetia simplex, Lotonus bainesli, Lotus spp., Macrotyloma axillare, Malus spp., Manihot esculenta, Medicago saliva, Metasequoia glyptostroboides, Musa sapientum, Nicotianum spp., Onobrychis spp., Ornithopus spp., Oryza spp., Peltophorum africanum, Pennisetum spp., Persea gratissima, Petunia spp., Phaseolus spp., Phoenix canariensis, Phormium cookianum, Photinia spp., Picea glauca, Pinus spp., Pisum sativam, Podocarpus totara, Pogonarthria fleckii, Pogonaffhria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys vefficillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, straw, sugar beet, sugar cane, sunflower, tomato, squash tea, maize, wheat, barley, rye, oat, peanut, pea, lentil and alfalfa, cotton, rapeseed, canola, pepper, sunflower, tobacco, eggplant, eucalyptus, a tree, an ornamental plant, a perennial grass and a forage crop. Alternatively algae and other non-Viridiplantae can be used for the methods of the present invention.

According to some embodiments of the invention, the plant used by the method of the invention is a crop plant including, but not limited to, cotton, Brassica vegetables, oilseed rape, sesame, olive tree, palm oil, banana, wheat, corn or maize, barley, alfalfa, peanuts, sunflowers, rice, oats, sugarcane, soybean, turf grasses, barley, rye, sorghum, sugar cane, chicory, lettuce, tomato, zucchini, bell pepper, eggplant, cucumber, melon, watermelon, beans, hibiscus, okra, apple, rose, strawberry, chili, garlic, pea, lentil, canola, mums, arabidopsis, broccoli, cabbage, beet, quinoa, spinach, squash, onion, leek, tobacco, potato, sugarbeet, papaya, pineapple, mango, Arabidopsis thaliana, and also plants used in horticulture, floriculture or forestry, such as, but not limited to, poplar, fir, eucalyptus, pine, an ornamental plant, a perennial grass and a forage crop, coniferous plants, moss, algae, as well as other plants listed in World Wide Web (dot) nationmaster (dot) com/encyclopedia/Plantae.

According to a specific embodiment of the present invention, the plant comprises a tomato.

The phrase “abiotic stress” as used herein refers to any adverse effect on metabolism, growth, viability and/or reproduction of a plant. Abiotic stress can be induced by any of suboptimal environmental growth conditions such as, for example, water deficit or drought, flooding, freezing, low or high temperature, strong winds, heavy metal toxicity, anaerobiosis, high or low nutrient levels (e.g. nutrient deficiency), high or low salt levels (e.g. salinity), atmospheric pollution, high or low light intensities (e.g. insufficient light) or UV irradiation. Abiotic stress may be a short term effect (e.g. acute effect, e.g. lasting for about a week) or alternatively may be persistent (e.g. chronic effect, e.g. lasting for example 10 days or more). The present invention contemplates situations in which there is a single abiotic stress condition or alternatively situations in which two or more abiotic stresses occur.

According to an embodiment, the abiotic stress refers to drought.

According to a specific embodiment, the drought is intermittent drought.

According to a specific embodiment, the drought is terminal drought.

Intermittent and terminal drought are the two distinct kinds of drought associated with limited rainfall that can be distinguished. Intermittent drought is due to climatic patterns of sporadic rainfall that causes intervals of drought and can occur at any time during the growing season or when the farmers have the option of irrigation but the supply is occasionally limited. In contrast, terminal drought occurs when plants suffer lack of water during later stages of reproductive growth or when crops are planted at the beginning of a dry season. In general, the lack of water interferes with the normal metabolism of the plant during flowering time and pod-fill, as these are stages when drought causes the greatest yield reduction.

As used herein the phrase “abiotic stress tolerance” refers to the ability of a plant to endure an abiotic stress without exhibiting substantial physiological or physical damage (e.g. alteration in metabolism, growth, viability and/or reproducibility of the plant).

As used herein the phrase “nitrogen use efficiency (NUE)” refers to a measure of crop production per unit of nitrogen fertilizer input. Fertilizer use efficiency (FUE) is a measure of NUE. Crop production can be measured by biomass, vigor or yield. The plant's nitrogen use efficiency is typically a result of an alteration in at least one of the uptake, spread, absorbance, accumulation, relocation (within the plant) and use of nitrogen absorbed by the plant. Improved NUE is with respect to that of a non-transgenic plant (i.e., lacking the transgene of the transgenic plant) of the same species and of the same developmental stage and grown under the same conditions.

As used herein the phrase “nitrogen-limiting conditions” refers to growth conditions which include a level (e.g., concentration) of nitrogen (e.g., ammonium or nitrate) applied which is below the level needed for optimal plant metabolism, growth, reproduction and/or viability.

As used herein the term/phrase “biomass”, “biomass of a plant” or “plant biomass” refers to the amount (e.g., measured in grams of air-dry tissue) of a tissue produced from the plant in a growing season. An increase in plant biomass can be in the whole plant or in parts thereof such as aboveground (e.g. harvestable) parts, vegetative biomass, roots and/or seeds or contents thereof (e.g., oil, starch etc.).

As used herein the term/phrase “vigor”, “vigor of a plant” or “plant vigor” refers to the amount (e.g., measured by weight) of tissue produced by the plant in a given time. Increased vigor could determine or affect the plant yield or the yield per growing time or growing area. In addition, early vigor (e.g. seed and/or seedling) results in improved field stand.

As used herein the term/phrase “yield”, “yield of a plant” or “plant yield” refers to the amount (e.g., as determined by weight or size) or quantity (e.g., numbers) of tissues or organs produced per plant or per growing season. Increased yield of a plant can affect the economic benefit one can obtain from the plant in a certain growing area and/or growing time.

According to one embodiment the yield is measured by cellulose content, oil content, starch content and the like.

According to another embodiment the yield is measured by oil content.

According to another embodiment the yield is measured by protein content.

According to another embodiment, the yield is measured by seed number per plant or part thereof (e.g., kernel, bean).

A plant yield can be affected by various parameters including, but not limited to, plant biomass; plant vigor; plant growth rate; seed yield; seed or grain quantity; seed or grain quality; oil yield; content of oil; starch and/or protein in harvested organs (e.g., seeds or vegetative parts of the plant); number of flowers (e.g. florets) per panicle (e.g. expressed as a ratio of number of filled seeds over number of primary panicles); harvest index; number of plants grown per area; number and size of harvested organs per plant and per area; number of plants per growing area (e.g. density); number of harvested organs in field; total leaf area; carbon assimilation and carbon partitioning (e.g. the distribution/allocation of carbon within the plant); resistance to shade; number of harvestable organs (e.g. seeds); seeds per pod; weight per seed; and modified architecture [such as increase stalk diameter, thickness or improvement of physical properties (e.g. elasticity)].

According to the present teachings, the plant has improved biomass, vigor and yield when grown under abiotic stress (e.g., drought).

As used herein the term “improving” or “increasing” refers to at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90% or greater increase in NUE, in tolerance to abiotic stress, in yield, in biomass or in vigor of a plant, as compared to a native or wild-type plants [i.e., plants not genetically modified to express the biomolecules (polynucleotides) of the invention, e.g., a non-transformed plant of the same species or a transformed plant transformed with a control vector, either of which being of the same developmental stage and grown under the same growth conditions as the transformed plant].

Improved plant NUE is translated in the field into either harvesting similar quantities of yield, while implementing less fertilizers, or increased yields gained by implementing the same levels of fertilizers. Thus, improved NUE or FUE has a direct effect on plant yield in the field.

In some embodiments, the expression of miR167 is only mildly elevated as compared to its native expression under normal growth conditions in order to achieve maximal tolerance and improved yield.

According to a specific embodiment, selection of such an expression pattern/level results in plants which exhibit a normal phenotype despite high yields/biomass/vigor under stress.

As used herein “a normal phenotype” refers to the overall plant phenotype of the wild-type plant under normal growth conditions.

Plant phenotype refers to plant complex traits such as growth, development, architecture, physiology, ecology, and the basic measurement of individual quantitative parameters that form the basis for the more complex traits. Examples for such direct measurement parameters are image-based projected leaf area, chlorophyll fluorescence, stem diameter, plant height/width, compactness, stress pigment concentration, tip burn, internode length, color, leaf angle, leaf rolling, leaf elongation, seed number, seed size, tiller number, flowering time, germination time etc.

Thus, according to an embodiment of the invention, the level of expression of total miR167 under the abiotic stress does not exceed 8 fold (e.g., 1.7-8) as compared to same in the plant when grown under the optimal conditions.

According to an embodiment of the invention, the level of expression of total miR167 under the abiotic stress does not exceed 5 fold (e.g., 1.7-5) as compared to same in the plant when grown under the optimal conditions.

According to an embodiment of the invention, the level of expression of total miR167 under the abiotic stress does not exceed 3 fold (e.g., 1.7-3) as compared to same in the plant when grown under the optimal conditions.

According to an embodiment of the invention, the level of expression of total miR167 under the abiotic stress does not exceed 2 fold as compared to same in the plant when grown under the optimal conditions.

According to an embodiment of the invention, the level of expression of total miR167 under the abiotic stress does not exceed 1.4-2 fold as compared to same in the plant when grown under the optimal conditions.

According to an embodiment of the invention, the level of expression of total miR167 under the abiotic stress does not exceed 1.7-2 fold as compared to same in the plant when grown under the optimal conditions.

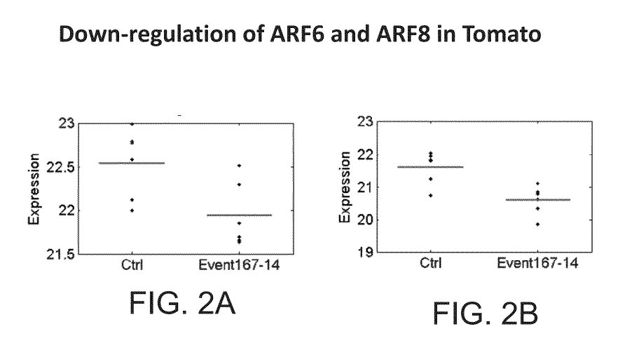

Measuring the level of gene expression is well known in the art. In the present case, miR167 expression or its precursor can be directly measured. As an alternative, measuring elevation in miR167 can be detected indirectly by measuring a decrease in at least one of its target genes e.g., ARF6 and ARF8, as illustrated in the Examples section which follows (see FIGS. 2A-B). The level of the target gene may be detected at the mRNA level or the protein level.

The expression level of the RNA in the cells of some embodiments of the invention can be determined using methods known in the art including, but not limited to, northern Blot analysis, RT-PCR analysis, RNA in situ hybridization stain, in situ RT-PCR stain and oligonucleotide microarray.

Additionally, the present inventors determine that the expression of total miR167 in the genetically modified plant under optimal conditions should be at the same level (equal) as that of miR167 in non-genetically modified plant of the same species being of the same developmental stage and growth conditions.

As used herein “total miR167” refers to endogenous miRNA167 expression and when applicable with the addition of miRNA167 resulting from an exogenous polynucleotide introduced into the cell.

As used herein “normal growth conditions” refers non-stress, optimal growth conditions. Such conditions, which depend on the plant being grown, are known to those skilled in the art of agriculture.

As used herein “same” refers to about identical with up to 20% deviation (increase or decrease), or less say, 10%, 5% or less say 1%.

As used herein, the phrase “microRNA (also referred to herein interchangeably as “miRNA” or “miR”) or a precursor thereof” refers to a microRNA (miRNA) molecule acting as a post-transcriptional regulator e.g., miR167. Typically, the miRNA molecules are RNA molecules of about 20 to 22 nucleotides in length which can be loaded into a RISC complex and which direct the cleavage of another RNA molecule, wherein the other RNA molecule comprises a nucleotide sequence essentially complementary to the nucleotide sequence of the miRNA molecule.

Typically, a miRNA molecule is processed from a “pre-miRNA” or as used herein a precursor of a pre-miRNA molecule by proteins, such as DCL proteins, present in any plant cell and loaded onto a RISC complex where it can guide the cleavage of the target RNA molecules.

Pre-microRNA molecules are typically processed from pri-microRNA molecules (primary transcripts). The single stranded RNA segments flanking the pre-microRNA are important for processing of the pri-miRNA into the pre-miRNA. The cleavage site appears to be determined by the distance from the stem-ssRNA junction (Han et al. 2006, Cell 125, 887-901, 887-901).

As used herein, a “pre-miRNA” molecule is an RNA molecule of about 100 to about 200 nucleotides, preferably about 100 to about 130 nucleotides which can adopt a secondary structure comprising a double stranded RNA stem and a single stranded RNA loop (also referred to as “hairpin”) and further comprising the nucleotide sequence of the miRNA (and its complement sequence) in the double stranded RNA stem. According to a specific embodiment, the miRNA and its complement are located about 10 to about 20 nucleotides from the free ends of the miRNA double stranded RNA stem. The length and sequence of the single stranded loop region are not critical and may vary considerably, e.g. between 30 and 50 nt in length. The complementarity between the miRNA and its complement need not be perfect and about 1 to 3 bulges of unpaired nucleotides can be tolerated. The secondary structure adopted by an RNA molecule can be predicted by computer algorithms conventional in the art such as mFOLD. The particular strand of the double stranded RNA stem from the pre-miRNA which is released by DCL activity and loaded onto the RISC complex is determined by the degree of complementarity at the 5′ end, whereby the strand, which at its 5′ end is the least involved in hydrogen bounding between the nucleotides of the different strands of the cleaved dsRNA stem, is loaded onto the RISC complex and will determine the sequence specificity of the target RNA molecule degradation. However, if empirically the miRNA molecule from a particular synthetic pre-miRNA molecule is not functional (because the “wrong” strand is loaded on the RISC complex), it will be immediately evident that this problem can be solved by exchanging the position of the miRNA molecule and its complement on the respective strands of the dsRNA stem of the pre-miRNA molecule. As is known in the art, binding between A and U involving two hydrogen bounds, or G and U involving two hydrogen bounds is less strong that between G and C involving three hydrogen bounds. Examples of hairpin sequences are provided in Tables 1-8, below.

Naturally occurring miRNA molecules may be comprised within their naturally occurring pre-miRNA molecules but they can also be introduced into existing pre-miRNA molecule scaffolds by exchanging the nucleotide sequence of the miRNA molecule normally processed from such existing pre-miRNA molecule for the nucleotide sequence of another miRNA of interest. The scaffold of the pre-miRNA can also be completely synthetic. Likewise, synthetic miRNA molecules may be comprised within, and processed from, existing pre-miRNA molecule scaffolds or synthetic pre-miRNA scaffolds. Some pre-miRNA scaffolds may be preferred over others for their efficiency to be correctly processed into the designed microRNAs, particularly when expressed as a chimeric gene wherein other DNA regions, such as untranslated leader sequences or transcription termination and polyadenylation regions are incorporated in the primary transcript in addition to the pre-microRNA.

According to the present teachings, the miRNA molecules may be naturally occurring or synthetic.

Thus, the present teachings contemplate expressing an exogenous polynucleotide having a nucleic acid sequence at least 90%, 91%, 92%, 93%, 94%, 95%, 96%, 97%, 98% 99% or 100% identical to SEQ ID NOs: 1, 4-369 (mature, see Tables 1 and 2 above), provided that they improve tolerance to abiotic stress.

Alternatively or additionally, the present teachings contemplate expressing an exogenous polynucleotide having a nucleic acid sequence at least 65%, 50%, 75%, 80%, 85%, 90%, 91%, 92%, 93%, 94%, 95%, 96%, 97%, 98% 99% or 100% identical to SEQ ID NOs: 1-735 (mature and precursors, see Tables 1 and 2 above), provided that they regulate abiotic stress tolerance of the plant.

The present invention envisages the use of homologous and orthologous sequences of the above miRNA molecules. At the precursor level use of homologous sequences can be done to a much broader extend. Thus, in such precursor sequences the degree of homology may be lower in all those sequences not including the mature miRNA segment therein.

Identity (e.g., percent identity) can be determined using any homology comparison software, including for example, the BlastN software of the National Center of Biotechnology Information (NCBI) such as by using default parameters.

Homology (e.g., percent homology, identity+similarity) can be determined using any homology comparison software, including for example, the TBLASTN software of the National Center of Biotechnology Information (NCBI) such as by using default parameters.

According to some embodiments of the invention, the term “homology” or “homologous” refers to identity of two or more nucleic acid sequences; or identity of two or more amino acid sequences.

Homologous sequences include both orthologous and paralogous sequences. The term “paralogous” relates to gene-duplications within the genome of a species leading to paralogous genes. The term “orthologous” relates to homologous genes in different organisms due to ancestral relationship.

One option to identify orthologues in monocot plant species is by performing a reciprocal blast search. This may be done by a first blast involving blasting the sequence-of-interest against any sequence database, such as the publicly available NCBI database which may be found at: Hypertext Transfer Protocol://World Wide Web (dot) ncbi (dot) nlm (dot) nih (dot) gov. The blast results may be filtered. The full-length sequences of either the filtered results or the non-filtered results are then blasted back (second blast) against the sequences of the organism from which the sequence-of-interest is derived. The results of the first and second blasts are then compared. An orthologue is identified when the sequence resulting in the highest score (best hit) in the first blast identifies in the second blast the query sequence (the original sequence-of-interest) as the best hit. Using the same rational a paralogue (homolog to a gene in the same organism) is found. In case of large sequence families, the ClustalW program may be used [Hypertext Transfer Protocol://World Wide Web (dot) ebi (dot) ac (dot) uk/Tools/clustalw2/index (dot) html], followed by a neighbor-joining tree (Hypertext Transfer Protocol://en (dot) wikipedia (dot) org/wiki/Neighbor-joining) which helps visualizing the clustering.

The present teachings refer to the expression of miR167 in an abiotic stress responsive manner.

As used herein “stress responsive” refers to the induction of expression only under an abiotic stress (e.g., drought) condition. Accordingly, under normal growth conditions (i.e., non-stress), there is no substantial change (i.e., same, as defined above) in miR167 levels as compared to a wild type plant of the same species, developmental stage and growth conditions.

According to one embodiment of the present invention, genetically modifying the plant to express miRNA167 is effected by expressing within the plant an exogenous polynucleotide encoding miR167.

As used herein, the phrase “exogenous polynucleotide” refers to a heterologous nucleic acid sequence which may not be naturally expressed within the plant or which overexpression [i.e., expression above that found in the control non-transformed plant (e.g., wild type) grown under the same conditions and being of the same developmental stage] in the plant is desired. The exogenous polynucleotide may be introduced into the plant in a stable or transient manner, so as to produce a ribonucleic acid (RNA) molecule. It should be noted that the exogenous polynucleotide may comprise a nucleic acid sequence which is identical or partially identical (homologous) to an endogenous nucleic acid sequence of the plant.

Generally, the recombinant DNA construct of this invention includes a promoter, functional in the cell in which the construct is intended to be transcribed, and operably linked to the DNA that undergoes processing to an RNA including single-stranded RNA that binds to the transcript of at least one target gene. In various embodiments, the promoter is selected from the group consisting of a constitutive promoter, a spatially specific promoter, a temporally specific promoter, a developmentally specific promoter, and an inducible promoter.

Non-constitutive promoters suitable for use with the recombinant DNA constructs of the invention include spatially specific promoters, temporally specific promoters, and inducible promoters. Spatially specific promoters can include organelle-, cell-, tissue-, or organ-specific promoters (e.g., a plastid-specific, a root-specific, a pollen-specific, or a seed-specific promoter for suppressing expression of the first target RNA in plastids, roots, pollen, or seeds, respectively). In many cases a seed-specific, embryo-specific, aleurone-specific, or endosperm-specific promoter is especially useful. Temporally specific promoters can include promoters that tend to promote expression during certain developmental stages in a plant's growth cycle, or during different times of day or night, or at different seasons in a year. Inducible promoters include promoters induced by chemicals or by environmental conditions such as, but not limited to, biotic or abiotic stress (e.g., water deficit or drought, heat, cold, high or low nutrient or salt levels, high or low light levels, or pest or pathogen infection). Of particular interest are microRNA promoters, especially those having a temporally specific, spatially specific, or inducible expression pattern; examples of miRNA promoters, as well as methods for identifying miRNA promoters having specific expression patterns, are provided in U.S. Patent Application Publication Nos. 2006/0200878, 2007/0199095, and 2007/0300329, which are specifically incorporated herein by reference. An expression-specific promoter can also include promoters that are generally constitutively expressed but at differing degrees or “strengths” of expression, including promoters commonly regarded as “strong promoters” or as “weak promoters”.

According to an embodiment of the invention the expression of the exogenous polynucleotide is under a stress-responsive promoter.

Stress responsive transcription factors in plants (e.g., Arabidopsis) are known to belong to AP2/EREBP, ABI3/VP1, ARF, bHLH, bZIP, HB, HSF, MYB, NAC and WRKY families of factors. STIFDB—Stress responsive Transcription Factor Database is a specialized database that provides information about various Stress responsive genes and Stress inducible Transcription Factor related information from Arabidopsis thaliana.

Non-limiting examples of abiotic stress-responsive promoters which can be used in accordance with the present teachings include, but are not limited to OsABA2, OsPrx, Wcor413, Lip5, and OsNAC6 (Gao et al 2008, Plant Cell Rep, 27(11):1787-95), XVSAP1 (Garwe et al 2003, J Exp Bot 54(381):191-201), and rab16A (Shiver et al 1991, PNAS 88:7266-7270), each of which is incorporated hereby by reference in its entirety.

According to a specific embodiment, the drought-responsive promoter is OsNAC6 (Ohnishi et al 2005, Genes Genet Syst 80(2):135-9, is incorporated hereby by reference in its entirety).

According to a specific embodiment, the drought-responsive promoter is not the hydroperoxide lyase promoter (e.g., of pORE-E2 vector).

In some embodiments, promoters of particular interest include the following examples: an opaline synthase promoter isolated from T-DNA of Agrobacterium; a cauliflower mosaic virus 35S promoter; enhanced promoter elements or chimeric promoter elements such as an enhanced cauliflower mosaic virus (CaMV) 35S promoter linked to an enhancer element (an intron from heat shock protein 70 of Zea mays); root specific promoters such as those disclosed in U.S. Pat. Nos. 5,837,848; 6,437,217 and 6,426,446; a maize L3 oleosin promoter disclosed in U.S. Pat. No. 6,433,252; a promoter for a plant nuclear gene encoding a plastid-localized aldolase disclosed in U.S. Patent Application Publication No. 2004/0216189; cold-inducible promoters disclosed in U.S. Pat. No. 6,084,089; salt-inducible promoters disclosed in U.S. Pat. No. 6,140,078; light-inducible promoters disclosed in U.S. Pat. No. 6,294,714; pathogen-inducible promoters disclosed in U.S. Pat. No. 6,252,138; and water deficit-inducible promoters disclosed in U.S. Patent Application Publication No. 2004/0123347 A1. All of the above-described patents and patent publications disclosing promoters and their use, especially in recombinant DNA constructs functional in plants are incorporated herein by reference.

In some embodiments, the DNA construct comprises a plant vascular- or phloem-specific promoter. Examples of plant vascular- or phloem-specific promoters include a rolC or rolA promoter of Agrobacterium rhizogenes, a promoter of a Agrobacterium tumefaciens T-DNA gene 5, the rice sucrose synthase RSs1 gene promoter, a Commelina yellow mottle badnavirus promoter, a coconut foliar decay virus promoter, a rice tungro bacilliform virus promoter, the promoter of a pea glutamine synthase GS3A gene, a invCD111 and invCD141 promoters of a potato invertase genes, a promoter isolated from Arabidopsis shown to have phloem-specific expression in tobacco by Kertbundit et al. (1991) Proc. Natl. Acad. Sci. USA., 88:5212-5216, a VAHOX1 promoter region, a pea cell wall invertase gene promoter, an acid invertase gene promoter from carrot, a promoter of a sulfate transporter gene Sultr1;3, a promoter of a plant sucrose synthase gene, and a promoter of a plant sucrose transporter gene.

In some embodiments, promoters suitable for use with a recombinant DNA construct of this invention include polymerase II (“pol II”) promoters and polymerase III (“pol III”) promoters. RNA polymerase II transcribes structural or catalytic RNAs that are usually shorter than 400 nucleotides in length, and recognizes a simple run of T residues as a termination signal; it has been used to transcribe siRNA duplexes (see, e.g., Lu et al. (2004) Nucleic Acids Res., 32:e171). Pol II promoters are therefore preferred in certain embodiments where a short RNA transcript is to be produced from a recombinant DNA construct of this invention. In one embodiment, the recombinant DNA construct includes a pol II promoter to express an RNA transcript flanked by self-cleaving ribozyme sequences (e.g., self-cleaving hammerhead ribozymes), resulting in a processed RNA, including single-stranded RNA that binds to the transcript of at least one target gene, with defined 5′ and 3′ ends, free of potentially interfering flanking sequences. An alternative approach uses pol III promoters to generate transcripts with relatively defined 5′ and 3′ ends, i.e., to transcribe an RNA with minimal 5′ and 3′ flanking sequences. In some embodiments, Pol III promoters (e.g., U6 or H1 promoters) are preferred for adding a short AT-rich transcription termination site that results in 2 base-pair overhangs (UU) in the transcribed RNA; this is useful, e.g., for expression of siRNA-type constructs. Use of pol III promoters for driving expression of siRNA constructs has been reported; see van de Wetering et al. (2003) EMBO Rep., 4: 609-615, and Tuschl (2002) Nature Biotechnol., 20: 446-448.

According to another embodiment, the level of miR167 is upregulated by expressing within the plant cell an exogenous polynucleotide encoding a positive regulator of miR167 in a stress responsive manner.

Alternatively or additionally, the level of miR167 is upregulated by expressing within the plant cell an exogenous polynucleotide which downregulates (e.g., dsRNA, RNAi spray, virus vectors, point mutations, zinc-finger protease) a negative regulator of miR167 in a stress-responsive manner.

Methods of expressing polynucleotides in plant cells are well known in the art.

Nucleic acid sequences of the polypeptides of some embodiments of the invention may be optimized for expression in a specific plant host. Examples of such sequence modifications include, but are not limited to, an altered G/C content to more closely approach that typically found in the plant species of interest, and the removal of codons atypically found in the plant species commonly referred to as codon optimization.

The phrase “codon optimization” refers to the selection of appropriate DNA nucleotides for use within a structural gene or fragment thereof that approaches codon usage within the plant of interest. Therefore, an optimized gene or nucleic acid sequence refers to a gene in which the nucleotide sequence of a native or naturally occurring gene has been modified in order to utilize statistically-preferred or statistically-favored codons within the plant. The nucleotide sequence typically is examined at the DNA level and the coding region optimized for expression in the plant species determined using any suitable procedure, for example as described in Sardana et al. (1996, Plant Cell Reports 15:677-681). In this method, the standard deviation of codon usage, a measure of codon usage bias, may be calculated by first finding the squared proportional deviation of usage of each codon of the native gene relative to that of highly expressed plant genes, followed by a calculation of the average squared deviation. The formula used is: 1 SDCU=n=1 N [(Xn−Yn)/Yn]2/N, where Xn refers to the frequency of usage of codon n in highly expressed plant genes, where Yn to the frequency of usage of codon n in the gene of interest and N refers to the total number of codons in the gene of interest. A table of codon usage from highly expressed genes of dicotyledonous plants is compiled using the data of Murray et al. (1989, Nuc Acids Res. 17:477-498).

One method of optimizing the nucleic acid sequence in accordance with the preferred codon usage for a particular plant cell type is based on the direct use, without performing any extra statistical calculations, of codon optimization tables such as those provided on-line at the Codon Usage Database through the NIAS (National Institute of Agrobiological Sciences) DNA bank in Japan (www (dot) kazusa (dot) or (dot) jp/codon/). The Codon Usage Database contains codon usage tables for a number of different species, with each codon usage table having been statistically determined based on the data present in Genbank.

By using the above tables to determine the most preferred or most favored codons for each amino acid in a particular species (for example, rice), a naturally-occurring nucleotide sequence encoding a protein of interest can be codon optimized for that particular plant species. This is effected by replacing codons that may have a low statistical incidence in the particular species genome with corresponding codons, in regard to an amino acid, that are statistically more favored. However, one or more less-favored codons may be selected to delete existing restriction sites, to create new ones at potentially useful junctions (5′ and 3′ ends to add signal peptide or termination cassettes, internal sites that might be used to cut and splice segments together to produce a correct full-length sequence), or to eliminate nucleotide sequences that may negatively effect mRNA stability or expression.

The naturally-occurring encoding nucleotide sequence may already, in advance of any modification, contain a number of codons that correspond to a statistically-favored codon in a particular plant species. Therefore, codon optimization of the native nucleotide sequence may comprise determining which codons, within the native nucleotide sequence, are not statistically-favored with regards to a particular plant, and modifying these codons in accordance with a codon usage table of the particular plant to produce a codon optimized derivative. A modified nucleotide sequence may be fully or partially optimized for plant codon usage provided that the protein encoded by the modified nucleotide sequence is produced at a level higher than the protein encoded by the corresponding naturally occurring or native gene. Construction of synthetic genes by altering the codon usage is described in for example PCT Patent Application 93/07278.

There are various methods of introducing foreign genes into both monocotyledonous and dicotyledonous plants (Potrykus, L, Annu. Rev. Plant. Physiol, Plant. MoI. Biol. (1991) 42:205-225; Shimamoto et al., Nature (1989) 338:274-276).

The principle methods of causing stable integration of exogenous DNA into plant genomic DNA include two main approaches:

(i) Agrobacterium-mediated gene transfer (e.g., T-DNA using Agrobacterium tumefaciens or Agrobacterium rhizogenes); see for example, Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S, and Arntzen, C. J., Butterworth Publishers, Boston, Mass. (1989) p. 93-112.

(ii) Direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; glass fibers or silicon carbide whisker transformation of cell cultures, embryos or callus tissue, U.S. Pat. No. 5,464,765 or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. See, e.g., Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledonous plants.

According to a specific embodiment of the present invention, the exogenous polynucleotide is introduced into the plant by infecting the plant with a bacteria, such as using a floral dip transformation method (as described in further detail in Example 5, of the Examples section which follows).

There are various methods of direct DNA transfer into plant cells. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues.

Following stable transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. For this reason it is preferred that the transformed plant be regenerated by micropropagation which provides a rapid, consistent reproduction of the transformed plants.

Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Although stable transformation is presently preferred, transient transformation of leaf cells, meristematic cells or the whole plant is also envisaged by the present invention.

Transient transformation can be effected by any of the direct DNA transfer methods described above or by viral infection using modified plant viruses.

Viruses that have been shown to be useful for the transformation of plant hosts include CaMV, Tobacco mosaic virus (TMV), brome mosaic virus (BMV) and Bean Common Mosaic Virus (BV or BCMV). Transformation of plants using plant viruses is described in U.S. Pat. No. 4,855,237 (bean golden mosaic virus; BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants are described in WO 87/06261. According to some embodiments of the invention, the virus used for transient transformations is avirulent and thus is incapable of causing severe symptoms such as reduced growth rate, mosaic, ring spots, leaf roll, yellowing, streaking, pox formation, tumor formation and pitting. A suitable avirulent virus may be a naturally occurring avirulent virus or an artificially attenuated virus. Virus attenuation may be effected by using methods well known in the art including, but not limited to, sub-lethal heating, chemical treatment or by directed mutagenesis techniques such as described, for example, by Kurihara and Watanabe (Molecular Plant Pathology 4:259-269, 2003).

Suitable virus strains can be obtained from available sources such as, for example, the American Type culture Collection (ATCC) or by isolation from infected plants. Isolation of viruses from infected plant tissues can be effected by techniques well known in the art such as described, for example by Foster and Tatlor, Eds. “Plant Virology Protocols From Virus Isolation to Transgenic Resistance (Methods in Molecular Biology (Humana Pr), VoI 81)”, Humana Press, 1998. Briefly, tissues of an infected plant believed to contain a high concentration of a suitable virus, preferably young leaves and flower petals, are ground in a buffer solution (e.g., phosphate buffer solution) to produce a virus infected sap which can be used in subsequent inoculations.

Construction of plant RNA viruses for the introduction and expression of non-viral exogenous polynucleotide sequences in plants is demonstrated by the above references as well as by Dawson, W. O. et al, Virology (1989) 172:285-292; Takamatsu et al. EMBO J. (1987) 6:307-311; French et al. Science (1986) 231:1294-1297; Takamatsu et al. FEBS Letters (1990) 269:73-76; and U.S. Pat. No. 5,316,931.

When the virus is a DNA virus, suitable modifications can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat proteins which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

In one embodiment, a plant viral nucleic acid is provided in which the native coat protein coding sequence has been deleted from a viral nucleic acid, a non-native plant viral coat protein coding sequence and a non-native promoter, preferably the subgenomic promoter of the non-native coat protein coding sequence, capable of expression in the plant host, packaging of the recombinant plant viral nucleic acid, and ensuring a systemic infection of the host by the recombinant plant viral nucleic acid, has been inserted. Alternatively, the coat protein gene may be inactivated by insertion of the non-native nucleic acid sequence within it, such that a protein is produced. The recombinant plant viral nucleic acid may contain one or more additional non-native subgenomic promoters. Each non-native subgenomic promoter is capable of transcribing or expressing adjacent genes or nucleic acid sequences in the plant host and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant under control of the subgenomic promoter to produce the desired products.

In a second embodiment, a recombinant plant viral nucleic acid is provided as in the first embodiment except that the native coat protein coding sequence is placed adjacent one of the non-native coat protein subgenomic promoters instead of a non-native coat protein coding sequence.

In a third embodiment, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that the sequences are transcribed or expressed in the host plant under control of the subgenomic promoters to produce the desired product.

In a fourth embodiment, a recombinant plant viral nucleic acid is provided as in the third embodiment except that the native coat protein coding sequence is replaced by a non-native coat protein coding sequence.

The viral vectors are encapsidated by the coat proteins encoded by the recombinant plant viral nucleic acid to produce a recombinant plant virus. The recombinant plant viral nucleic acid or recombinant plant virus is used to infect appropriate host plants. The recombinant plant viral nucleic acid is capable of replication in the host, systemic spread in the host, and transcription or expression of foreign gene(s) (isolated nucleic acid) in the host to produce the desired protein.

In addition to the above, the nucleic acid molecule of the present invention can also be introduced into a chloroplast genome thereby enabling chloroplast expression.

A technique for introducing exogenous nucleic acid sequences to the genome of the chloroplasts is known. This technique involves the following procedures. First, plant cells are chemically treated so as to reduce the number of chloroplasts per cell to about one. Then, the exogenous nucleic acid is introduced via particle bombardment into the cells with the aim of introducing at least one exogenous nucleic acid molecule into the chloroplasts. The exogenous nucleic acid is selected such that it is integratable into the chloroplast's genome via homologous recombination which is readily effected by enzymes inherent to the chloroplast. To this end, the exogenous nucleic acid includes, in addition to a gene of interest, at least one nucleic acid stretch which is derived from the chloroplast's genome. In addition, the exogenous nucleic acid includes a selectable marker, which serves by sequential selection procedures to ascertain that all or substantially all of the copies of the chloroplast genomes following such selection will include the exogenous nucleic acid. Further details relating to this technique are found in U.S. Pat. Nos. 4,945,050; and 5,693,507 which are incorporated herein by reference. A polypeptide can thus be produced by the protein expression system of the chloroplast and become integrated into the chloroplast's inner membrane.

Once the plant is obtained it is allowed to grow under the abiotic stress. However growth under normal conditions is also contemplated according to the present teachings.

Based on the present teachings the present inventors have generated a plant or a plant cell genetically modified to express miR167, wherein expression of the miRNA167 in the plant cell is abiotic stress responsive and further wherein a level of expression of total miR167 in the plant cell under the abiotic stress does not exceed 10 fold as compared to same in a plant when grown under optimal conditions.

Methods of qualifying plants as being tolerant or having improved tolerance to abiotic stress or limiting nitrogen levels are well known in the art and are further described hereinbelow.

Fertilizer use efficiency—To analyze whether the transgenic plants are more responsive to fertilizers, plants are grown in agar plates or pots with a limited amount of fertilizer, as described, for example, in Yanagisawa et al (Proc Natl Acad Sci US A. 2004; 101:7833-8). The plants are analyzed for their overall size, time to flowering, yield, protein content of shoot and/or grain. The parameters checked are the overall size of the mature plant, its wet and dry weight, the weight of the seeds yielded, the average seed size and the number of seeds produced per plant. Other parameters that may be tested are: the chlorophyll content of leaves (as nitrogen plant status and the degree of leaf verdure is highly correlated), amino acid and the total protein content of the seeds or other plant parts such as leaves or shoots, oil content, etc. Similarly, instead of providing nitrogen at limiting amounts, phosphate or potassium can be added at increasing concentrations. Again, the same parameters measured are the same as listed above. In this way, nitrogen use efficiency (NUE), phosphate use efficiency (PUE) and potassium use efficiency (KUE) are assessed, checking the ability of the transgenic plants to thrive under nutrient restraining conditions.

Nitrogen use efficiency—To analyze whether the transgenic plants (e.g., Arabidopsis plants) are more responsive to nitrogen, plant are grown in 0.75-3 millimolar (mM, nitrogen deficient conditions) or 10, 6-9 mM (optimal nitrogen concentration). Plants are allowed to grow for additional 25 days or until seed production. The plants are then analyzed for their overall size, time to flowering, yield, protein content of shoot and/or grain/seed production. The parameters checked can be the overall size of the plant, wet and dry weight, the weight of the seeds yielded, the average seed size and the number of seeds produced per plant. Other parameters that may be tested are: the chlorophyll content of leaves (as nitrogen plant status and the degree of leaf greenness is highly correlated), amino acid and the total protein content of the seeds or other plant parts such as leaves or shoots and oil content. Transformed plants not exhibiting substantial physiological and/or morphological effects, or exhibiting higher measured parameters levels than wild-type plants, are identified as nitrogen use efficient plants.

Nitrogen Use efficiency assay using plantlets—The assay is done according to Yanagisawa-S. et al. with minor modifications (“Metabolic engineering with Dofl transcription factor in plants: Improved nitrogen assimilation and growth under low-nitrogen conditions” Proc. Natl. Acad. Sci. USA 101, 7833-7838). Briefly, transgenic plants which are grown for 7-10 days in 0.5×MS [Murashige-Skoog] supplemented with a selection agent are transferred to two nitrogen-limiting conditions: MS media in which the combined nitrogen concentration (NH₄NO₃ and KNO₃) was 0.75 mM (nitrogen deficient conditions) or 6-15 mM (optimal nitrogen concentration). Plants are allowed to grow for additional 30-40 days and then photographed, individually removed from the Agar (the shoot without the roots) and immediately weighed (fresh weight) for later statistical analysis. Constructs for which only T1 seeds are available are shown on selective media and at least 20 seedlings (each one representing an independent transformation event) are carefully transferred to the nitrogen-limiting media. For constructs for which T2 seeds are available, different transformation events are analyzed. Usually, 20 randomly selected plants from each event are transferred to the nitrogen-limiting media allowed to grow for 3-4 additional weeks and individually weighed at the end of that period. Transgenic plants are compared to control plants grown in parallel under the same conditions. Mock-transgenic plants expressing the uidA reporter gene (GUS) under the same promoter or transgenic plants carrying the same promoter but lacking a reporter gene are used as control.

Nitrogen determination—The procedure for N (nitrogen) concentration determination in the structural parts of the plants involves the potassium persulfate digestion method to convert organic N to NO₃⁻ (Purcell and King 1996 Argon. J. 88:111-113, the modified Cd⁻ mediated reduction of NO₃⁻ to NO₂⁻ (Vodovotz 1996 Biotechniques 20:390-394) and the measurement of nitrite by the Griess assay (Vodovotz 1996, supra). The absorbance values are measured at 550 nm against a standard curve of NaNO₂. The procedure is described in details in Samonte et al. 2006 Agron. J. 98:168-176.

Tolerance to abiotic stress (e.g. tolerance to drought or salinity) can be evaluated by determining the differences in physiological and/or physical condition, including but not limited to, vigor, growth, size, or root length, or specifically, leaf color or leaf area size of the transgenic plant compared to a non-modified plant of the same species grown under the same conditions. Other techniques for evaluating tolerance to abiotic stress include, but are not limited to, measuring chlorophyll fluorescence, photosynthetic rates and gas exchange rates. Further assays for evaluating tolerance to abiotic stress are provided hereinbelow and in the Examples section which follows.

Drought tolerance assay—Soil-based drought screens are performed with plants overexpressing the polynucleotides detailed above. Seeds from control Arabidopsis plants, or other transgenic plants overexpressing nucleic acid of the invention are germinated and transferred to pots. Drought stress is obtained after irrigation is ceased. Transgenic and control plants are compared to each other when the majority of the control plants develop severe wilting. Plants are re-watered after obtaining a significant fraction of the control plants displaying a severe wilting. Plants are ranked comparing to controls for each of two criteria: tolerance to the drought conditions and recovery (survival) following re-watering.

Quantitative parameters of tolerance measured include, but are not limited to, the average wet and dry weight, growth rate, leaf size, leaf coverage (overall leaf area), the weight of the seeds yielded, the average seed size and the number of seeds produced per plant. Transformed plants not exhibiting substantial physiological and/or morphological effects, or exhibiting higher biomass than wild-type plants, are identified as drought stress tolerant plants.

Salinity tolerance assay—Transgenic plants with tolerance to high salt concentrations are expected to exhibit better germination, seedling vigor or growth in high salt. Salt stress can be effected in many ways such as, for example, by irrigating the plants with a hyperosmotic solution, by cultivating the plants hydroponically in a hyperosmotic growth solution (e.g., Hoagland solution with added salt), or by culturing the plants in a hyperosmotic growth medium [e.g., 50% Murashige-Skoog medium (MS medium) with added salt]. Since different plants vary considerably in their tolerance to salinity, the salt concentration in the irrigation water, growth solution, or growth medium can be adjusted according to the specific characteristics of the specific plant cultivar or variety, so as to inflict a mild or moderate effect on the physiology and/or morphology of the plants (for guidelines as to appropriate concentration see, Bernstein and Kafkafi, Root Growth Under Salinity Stress In: Plant Roots, The Hidden Half 3rd ed. Waisel Y, Eshel A and Kafkafi U. (editors) Marcel Dekker Inc., New York, 2002, and reference therein).

For example, a salinity tolerance test can be performed by irrigating plants at different developmental stages with increasing concentrations of sodium chloride (for example 50 mM, 150 mM, 300 mM NaCl) applied from the bottom and from above to ensure even dispersal of salt. Following exposure to the stress condition the plants are frequently monitored until substantial physiological and/or morphological effects appear in wild type plants. Thus, the external phenotypic appearance, degree of chlorosis and overall success to reach maturity and yield progeny are compared between control and transgenic plants. Quantitative parameters of tolerance measured include, but are not limited to, the average wet and dry weight, growth rate, leaf size, leaf coverage (overall leaf area), the weight of the seeds yielded, the average seed size and the number of seeds produced per plant. Transformed plants not exhibiting substantial physiological and/or morphological effects, or exhibiting higher biomass than wild-type plants, are identified as abiotic stress tolerant plants.

Osmotic tolerance test—Osmotic stress assays (including sodium chloride and PEG assays) are conducted to determine if an osmotic stress phenotype was sodium chloride-specific or if it was a general osmotic stress related phenotype. Plants which are tolerant to osmotic stress may have more tolerance to drought and/or freezing. For salt and osmotic stress experiments, the medium is supplemented for example with 50 mM, 100 mM, 200 mM NaCl or 15%, 20% or 25% PEG.

Cold stress tolerance—One way to analyze cold stress is as follows. Mature (25 day old) plants are transferred to 4° C. chambers for 1 or 2 weeks, with constitutive light. Later on plants are moved back to greenhouse. Two weeks later damages from chilling period, resulting in growth retardation and other phenotypes, are compared between control and transgenic plants, by measuring plant weight (wet and dry), and by comparing growth rates measured as time to flowering, plant size, yield, and the like.

Heat stress tolerance—One way to measure heat stress tolerance is by exposing the plants to temperatures above 34° C. for a certain period. Plant tolerance is examined after transferring the plants back to 22° C. for recovery and evaluation after 5 days relative to internal controls (non-transgenic plants) or plants not exposed to neither cold or heat stress.

The biomass, vigor and yield of the plant can also be evaluated using any method known to one of ordinary skill in the art. Thus, for example, plant vigor can be calculated by the increase in growth parameters such as leaf area, fiber length, rosette diameter, plant fresh weight, oil content, seed yield and the like per time.

As mentioned, the increase of plant yield can be determined by various parameters. For example, increased yield of rice may be manifested by an increase in one or more of the following: number of plants per growing area, number of panicles per plant, number of spikelets per panicle, number of flowers per panicle, increase in the seed filling rate, increase in thousand kernel weight (1000-weight), increase oil content per seed, increase starch content per seed, among others. An increase in yield may also result in modified architecture, or may occur because of modified architecture. Similarly, increased yield of soybean may be manifested by an increase in one or more of the following: number of plants per growing area, number of pods per plant, number of seeds per pod, increase in the seed filling rate, increase in thousand seed weight (1000-weight), reduce pod shattering, increase oil content per seed, increase protein content per seed, among others. An increase in yield may also result in modified architecture, or may occur because of modified architecture.

Thus, the present invention is of high agricultural value for increasing tolerance of plants to nitrogen deficiency or abiotic stress as well as promoting the yield, biomass and vigor of commercially desired crops.

According to another embodiment of the present invention, there is provided a food or feed comprising the plants or a portion thereof of the present invention.

In a further aspect the invention, the transgenic plants of the present invention or parts thereof are comprised in a food or feed product (e.g., dry, liquid, paste). A food or feed product is any ingestible preparation containing the transgenic plants, or parts thereof, of the present invention, or preparations made from these plants. Thus, the plants or preparations are suitable for human (or animal) consumption, i.e. the transgenic plants or parts thereof are more readily digested. Feed products of the present invention further include an oil or a beverage adapted for animal consumption.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

A DNA fragment encoding the hairpin of the Arabidopsis microRNA 167a (SEQ ID NO: 3, see Table 1 above) was cloned into the pORE-E2 vector between the Bam HI and KpnI restriction sites.

MicroRNA 167 was expressed under the regulation of the hydroperoxide lyase promoter (HPL). The vector was named pORE167a-E2 and used to transform tomato plants cultivar M82 using the Agrobacterium-mediated transformation method.

Transgenic events were selected on media containing 50 μg/μl kanamycin and antibiotic-resistant events were selected. The presence of pORE167a-E2 in the transgenic plants was verified by PCR and three events were selected for further analysis: Event number 7, 14 and 21.

Expression of microRNA 167a was tested in the transgenic events using qRT-PCR compared to a control, which was transformed with the pORE-E2 empty vector.

The expression was tested using samples taken from plants grown under optimal irrigation and drought stress. No significant change in the expression level was detected under optimal irrigation and an increase of up to 2-fold was detected under drought stress.

MicroRNA 167a is known to regulate two Auxin Responsive Factor genes: ARF6 and ARF8. Therefore, the expression level of ARF6 and ARF8 was tested in the transgenic lines compared to the empty vector control and found to be mildly down regulated.

Next, the ability of a mild increased expression of microRNA 167a to improve yield of tomato plants grown under drought stress was tested. The three transgenic events and the empty vector control were grown in a growth chamber at 24° C. with a 16 hours light: 8 hours dark regime. Each group of either transgenic event or control consisted of 8 plants. The plants were initially grown for 4 weeks under optimal irrigation conditions (plants were irrigated to saturation twice a week). At the end of four weeks the plants started to produce flowers and a two-week drought period was applied. After the two weeks of drought the plants were recovered by irrigation to saturation twice a week for two weeks and a second drought period of one week was applied. After the second drought period the plants were recovered and maintained on optimal irrigation until the end of the experiment. Tomato fruits were collected and weighed from the plants as the fruit ripened. The total fruit weight produced by the 8 control plants and the 8 plants of each of the transgenic events is presented in the following table (Table 3A, below). This experiment was repeated with the same transgenic events grown again under drought during flowering conditions, similarly to what was described above. The yield obtained by the transgenic events and the control is presented in Table 3B, below.

TABLE 3A

| Time after | | Total fruit | Total fruit | Total fruit |
|---|---|---|---|---|
| beginning | Total fruit | weight in 8 | weight in 8 | weight in 8 |
| of the | weight in 8 | plants of | plants of | plants of |
| experiment | control plants | event#7 | event#14 | event#21 |
| 4 months | 113 grams | 985 grams | 831 grams | 686 grams |
| 4.5 months | 144 grams | 1,611 grams | 1,357 grams | 1,231 grams |
| 5 months | 216 grams | 2,082 grams | 1,903 grams | 1,792 grams |

TABLE 3B

| Time after | | Total fruit | Total fruit | Total fruit |
|---|---|---|---|---|
| beginning | Total fruit | weight in 8 | weight in 8 | weight in 8 |
| of the | weight in 8 | plants of | plants of | plants of |
| experiment | control plants | event#7 | event#14 | event#21 |
| 3 months | 127 grams | 537 grams | 318 grams | 280 grams |

Down-regulation of miR167 target genes, ARF6 and ARF8, was observed in transgenic tomato plants expressing miR167. See FIG. 2A, which shows Sly-ARF6 down-regulation compared to control, p-value=0.022, fold change of 1.87. FIG. 2B shows Sly-ARF8 down-regulation compared to control, p-value=0.0045, fold change of 2.17. The results are indicative of total miR167 level in the transgenic plants.

### Example 2

The yield of the three transgenic events described in Example 1 was further tested compared to a control plant expressing the empty vector under heat stress conditions, as follows: The three transgenic events and the empty vector control were initially grown in a growth chamber at 24° C. with a 16 hours light:8 hours dark regime. Optimal irrigation was applied throughout the experiment. Each group of either transgenic event or control consisted of 10 plants. The plants were initially grown for 4 weeks under optimal temperature (24° C.). At the end of four weeks the plants started to produce flowers and a first heat stress was applied for three days, 3 hours of stress per day at 35-40° C. After the heat stress, the plants were recovered by returning to optimal temperature of 24° C. for two weeks. Following this recovery time, a second heat-stress was applied, similarly to the first heat stress (3 days, 3 hours per day at 35-40° C.). After the second heat stress, the plants were recovered and maintained at optimal temperature until the end of the experiment. Tomato fruits were collected and weighed from the plants as the fruit ripened. The total fruit weight produced by the 10 control plants and the 10 plants of each of the transgenic events is presented in Table 4 below.

TABLE 4

| Time after | | Total fruit | Total fruit | Total fruit |
|---|---|---|---|---|
| beginning | Total fruit | weight in 8 | weight in 8 | weight in 8 |
| of the | weight in 8 | plants of | plants of | plants of |
| experiment | control plants | event#7 | event#14 | event#21 |
| 3.5 months | 884 grams | 1782 grams | 2017 grams | 1823 grams |

### Example 3

The yield of the three transgenic events as described in Example 1 was also tested compared to a control plant expressing the empty vector under optimal conditions, as follows:

The three transgenic event plants and the empty vector control plants were grown in a growth chamber at 24° C. with a 16 hours light: 8 hours dark regime and optimal irrigation throughout the experiment. Each group of either transgenic event or control consisted of 8 plants. Tomato fruits were collected and weighed from the plants as the fruit ripened.

The total fruit weight produced by the 8 control plants and the 8 plants of each of the transgenic events is presented in Table 5:

TABLE 5

| Time after | | Total fruit | Total fruit | Total fruit |
|---|---|---|---|---|
| beginning | Total fruit | weight in 8 | weight in 8 | weight in 8 |
| of the | weight in 8 | plants of | plants of | plants of |
| experiment | control plants | event#7 | event#14 | event#21 |
| 4 months | 604 grams | 777 grams | 1758 grams | 1454 grams |

### Example 4

This example illustrates a method of improving abiotic stress tolerance of maize plants. More specifically, this example describes a non-limiting method of providing a maize plant that transgenically expresses a miR167 and exhibits improved yield under abiotic stress conditions (e.g., drought, temperature, or salt stress) in comparison to a control plant that does not transgenically express the miR167.

Transformation vectors for use in making recombinant DNA constructs for Agrobacterium-mediated transformation of maize cells are known in the art; a non-limiting example is the base transformation vector pMON93039 (described as the vector having SEQ ID NO: 2065 and illustrated in Table 4 and FIG. 2 of U.S. Patent Application Publication No. 2011/0296555 (U.S. patent application Ser. No. 12/999,777 published 1 Dec. 2011), incorporated by reference herein. A transformation vector for the transgenic expression of a mature miR167 (ath-miR167a, SEQ ID NO:1; see Table 1) is constructed using methods as described in U.S. Patent Application Publication No. 2011/0296555 by inserting an expression cassette including a promoter functional in a maize plant cell operably linked to a polynucleotide encoding a miR167 stem-loop precursor (ath-miR167a precursor, SEQ ID NO:2; see Table 1) at an insertion site, e.g., between the intron element (coordinates 1287-1766) and the polyadenylation element (coordinates 1838-2780) of the base vector pMON93039. The promoter can be any promoter functional in a maize plant cell, such as a constitutive promoter, a meristem promoter, a root promoter, an ovule promoter, a pollen promoter, or a stress-enhanced promoter, such as a drought-inducible promoter or injury-inducible promoter. Non-limiting examples of specific promoters include an Os.Gos2 constitutive promoter (SEQ ID NO: 736, a Zm.H2a meristem promoter (SEQ ID NO: 737), and an Os.RAB17 drought-inducible promoter (SEQ ID NO: 738). The expression cassette optionally includes other elements, e.g., 5′ leader or 3′ terminator sequences, and can be stacked with expression cassettes for expressing other genes of interest such as protein-coding sequences.

For Agrobacterium-mediated transformation of maize embryo cells, maize plants of a transformable line are grown in the greenhouse and ears are harvested when the embryos are 1.5 to 2.0 mm in length. Ears are surface sterilized by spraying or soaking the ears in 80% ethanol, followed by air drying. Immature embryos are isolated from individual kernels from sterilized ears. Prior to inoculation of maize cells, cultures of Agrobacterium containing a transformation vector for expressing an expression cassette including a promoter functional in a maize plant cell operably linked to a polynucleotide encoding the ath-miR167a precursor, SEQ ID NO:2 as described above are grown overnight at room temperature. Immature maize embryo cells are inoculated with Agrobacterium after excision, incubated at room temperature with Agrobacterium for 5 to 20 minutes, and then co-cultured with Agrobacterium for 1 to 3 days at 23 degrees Celsius in the dark. Co-cultured embryos are transferred to a selection medium and cultured for approximately two weeks to allow embryogenic callus to develop. Embryogenic callus is transferred to a culture medium containing 100 mg/L paromomycin and subcultured at about two week intervals. Multiple events of transformed plant cells are recovered 6 to 8 weeks after initiation of selection. Transgenic maize plants are regenerated from transgenic plant cell callus for each of the multiple transgenic events resulting from transformation and selection. The callus of transgenic plant cells of each event is placed on a medium to initiate shoot and root development into plantlets which are transferred to potting soil for initial growth in a growth chamber at 26 degrees Celsius, followed by growth on a mist bench before transplanting to pots where plants are grown to maturity. The regenerated plants are self-fertilized. First generation (“R1”) seed is harvested. The seed or plants grown from the seed is used to select seeds, seedlings, progeny second generation (“R2”) transgenic plants, or hybrids, e.g., by selecting transgenic plants exhibiting an enhanced trait as compared to a control plant (a plant lacking expression of the recombinant DNA construct).

Additional individual transformation vectors for the transgenic expression of mature miRNAs with the homologue sequences provided in Table 2 are similarly constructed by inserting an expression cassette including a promoter functional in a maize plant cell operably linked at least one polynucleotide encoding a miR167 stem-loop precursor having a sequence selected from the hairpin SEQ ID NOs provided in Table 2 into an insertion site of a base transformation vector. The Agrobacterium-mediated transformation process is repeated with these additional transformation vectors to produce multiple events of transgenic maize plants each transgenically expressing a mature miR167. Transgenic plant regeneration and production from these transformation events is carried out as described above and screened for improved yield under broad acre field conditions, including under normal water and nutrient conditions or under abiotic stress conditions (drought, temperature, salt stress, nutrient stress). Transgenic plants are also screened for enhanced pollen viability, and for improved fruit or seed set. Transgenic plants are also screened for down-regulation of miR167 target genes, ARF6 and ARF8. The levels of the miR167 target genes, ARF6 and ARF8, in the transgenic plants are indicative of total miR167 level. Plants expressing a desired level (for example about 2, 3, 4, 5, 6, 7, 8, 9, or 10 fold increased levels), of miRNA167 are selected.

Generally, screening a population of transgenic plants each regenerated from a transgenic plant cell is performed to identify transgenic plant cells that develop into transgenic plants having the desired trait. The transgenic plants are assayed to detect an enhanced trait, e.g., enhanced water use efficiency, enhanced cold tolerance, increased yield, enhanced nitrogen use efficiency, enhanced seed protein, and enhanced seed oil. Screening methods include direct screening for the trait in a greenhouse or field trial or screening for a surrogate trait. Such analyses are directed to detecting changes in the chemical composition, biomass, physiological properties, or morphology of the plant. Changes in chemical compositions such as nutritional composition of grain are detected by analysis of the seed composition and content of protein, free amino acids, oil, free fatty acids, starch, tocopherols, or other nutrients. Changes in growth or biomass characteristics are detected by measuring plant height, stem diameter, internode length, root and shoot dry weights, and (for grain-producing plants such as maize, rice, or wheat) ear or seed head length and diameter. Changes in physiological properties are identified by evaluating responses to stress conditions, e.g., assays under imposed stress conditions such as water deficit, nitrogen or phosphate deficiency, cold or hot growing conditions, pathogen or insect attack, light deficiency, or increased plant density. Other selection properties include days to pollen shed, days to silking in maize, leaf extension rate, chlorophyll content, leaf temperature, stand, seedling vigor, internode length, plant height, leaf number, leaf area, tillering, brace roots, staying green, stalk lodging, root lodging, plant health, fertility, green snap, and pest resistance. In addition, phenotypic characteristics of harvested seed may be evaluated; for example, in maize this can include the number of kernels per row on the ear, number of rows of kernels on the ear, kernel abortion, kernel weight, kernel size, kernel density and physical grain quality.

The following paragraphs illustrate non-limiting examples of screening assays useful for identifying desired traits in maize plants. These assays can be readily adapted for screening other plants such as canola, cotton, soybean, or vegetables such as tomato, either as hybrids or inbreds.

(A) Transgenic maize plants having enhanced yield are identified from the transgenic maize plants prepared as described above by screening the transgenic plants over multiple locations with plants grown under optimal production management practices and maximum weed and pest control. A useful target for improved yield is a 5% to 10% increase in yield as compared to yield produced by plants grown from seed for a control plant. Selection methods may be applied in multiple and diverse geographic locations and over one or more planting seasons to statistically distinguish yield improvement from natural environmental effects. Transgenic maize plants having enhanced yield under drought or water-stress conditions are identified in a similar manner by screening the transgenic plants under different water regimes.

(B) Transgenic maize plants having enhanced water use efficiency are identified by screening plants in an assay where water is withheld for period to induce stress followed by watering to revive the plants. For example, a useful selection process imposes 3 drought/re-water cycles on plants over a total period of 15 days after an initial stress-free growth period of 11 days. Each cycle consists of 5 days, with no water being applied for the first four days and a water quenching on the 5th day of the cycle. The primary phenotypes analyzed by the selection method are the changes in plant growth rate as determined by height and biomass during a vegetative drought treatment.

(C) Transgenic maize plants having nitrogen use efficiency are identified by screening in fields with three levels of nitrogen fertilizer being applied, e.g., low level (0 pounds/acre), medium level (80 pounds/acre) and high level (180 pounds/acre). Plants with enhanced nitrogen use efficiency provide higher yield as compared to control plants.

(D) Transgenic maize plants having enhanced cold tolerance are identified by screening plants in a cold germination assay and/or a cold tolerance field trial. In a cold germination assay trays of transgenic and control seeds are placed in a dark growth chamber at 9.7 degrees Celsius for 24 days. Seeds having higher germination rates as compared to the control are identified as having enhanced cold tolerance. In a cold tolerance field trial plants with enhanced cold tolerance are identified from field planting at an earlier date than conventional spring planting for the field location. For example, seeds are planted into the ground around two weeks before local farmers begin to plant maize so that a significant cold stress is exerted onto the crop. As a control, seeds also are planted under local optimal planting conditions such that the crop has little or no exposure to cold condition. At each location, seeds are planted under both cold and normal conditions preferably with multiple repetitions per treatment.

### Example 5

This example illustrates a method of improving abiotic stress tolerance of soybean plants. More specifically this example describes a non-limiting method of providing a soybean plant that transgenically expresses a miR167 and exhibits improved yield under abiotic stress conditions (e.g., drought, temperature, or salt stress) in comparison to a control plant that does not transgenically express the miR 167.

Transformation vectors for use in making recombinant DNA constructs for Agrobacterium-mediated transformation of soybean cells are known in the art; a non-limiting example is the base transformation vector pMON82053 (described as the vector having SEQ ID NO: 2066 and illustrated in Table 7 and FIG. 3 of U.S. Patent Application Publication No. 2011/0296555 (U.S. application Ser. No. 12/999,777 published 1 Dec. 2011), incorporated by reference herein. A transformation vector for the transgenic expression of a mature miR167 (ath-miR167a, SEQ ID NO:1; see Table 1) is constructed using methods as described in U.S. Patent Application Publication No. 2011/0296555 by inserting an expression cassette including a promoter functional in a soybean plant cell operably linked to a polynucleotide encoding a miR167 stem-loop precursor (ath-miR167a precursor, SEQ ID NO:2; see Table 1) at an insertion site, e.g., between the intron element (coordinates 1287-1766) and the polyadenylation element (coordinates 1838-2780) of the base vector pMON82053. The promoter can be any promoter functional in a soybean plant cell, such as a constitutive promoter, a meristem promoter, a root promoter, an ovule promoter, a pollen promoter, or a stress-enhanced promoter, such as a drought-inducible promoter or injury-inducible promoter. The expression cassette optionally includes other elements, e.g., a terminator, and can be stacked with expression cassettes for expressing other genes of interest.

For Agrobacterium-mediated transformation, soybean seeds are imbided overnight and the meristem explants excised and placed in a wounding vessel. Cultures of induced Agrobacterium containing a transformation vector for expressing an expression cassette including a promoter functional in a soybean plant cell operably linked to a polynucleotide encoding the ath-miR167a precursor, SEQ ID NO:2 as described above are mixed with prepared explants. Inoculated explants are wounded using sonication, placed in co-culture for 2-5 days, and transferred to selection media for 6-8 weeks to allow selection and growth of transgenic shoots. Resistant shoots are harvested at approximately 6-8 weeks and placed into selective rooting media for 2-3 weeks. Shoots producing roots are transferred to the greenhouse and potted in soil.

Additional individual transformation vectors for the transgenic expression of mature miRNAs with the homologue sequences provided in Table 2 are similarly constructed by inserting an expression cassette including a promoter functional in a soybean plant cell operably linked at least one polynucleotide encoding a miR167 stem-loop precursor having a sequence selected from the hairpin SEQ ID NOs provided in Table 2 into an insertion site of a base transformation vector. The Agrobacterium-mediated transformation process is repeated with these additional transformation vectors to produce multiple events of transgenic soybean plants each transgenically expressing a mature miR167. Transgenic plant regeneration and production from these transformation events is carried out as described above and screened for improved yield under broad acre field conditions, including under normal water and nutrient conditions or under abiotic stress conditions (drought, temperature, salt stress, nutrient stress). Transgenic plants are also screened for enhanced pollen viability, and for improved fruit or seed set. Transgenic plants are also screened for down-regulation of miR167 target genes, ARF6 and ARF8. The levels of the miR167 target genes, ARF6 and ARF8, in the transgenic soybean plants are indicative of total miR167 level. Soybean plants expressing a desired level (for example about 2, 3, 4, 5, 6, 7, 8, 9, or 10 fold increased levels), of miRNA167 are selected. Screening methods are similar to those described in Example 4 for maize plants.

The regenerated transgenic soybean plants, or progeny transgenic soybean plants or soybean seeds, produced from the regenerated transgenic soybean plants, are screened for an enhanced trait (e.g., increased yield under sufficient water conditions or increased yield under drought or water-stress conditions), as compared to a control plant or seed (a plant or seed lacking expression of the recombinant DNA construct). From each group of multiple events of transgenic soybean plants with a specific recombinant construct of this invention, the event that produces the greatest enhanced trait (e.g., greatest enhancement in yield) is identified and progeny soybean seed is selected for commercial development.

### Example 6

This example illustrates a method of providing transgenic rootstock for improving yields in grafted plants. More specifically, this example describes a non-limiting method of providing a solanaceous plant rootstock that transgenically expresses a miR167 and is useful in making grafted plants exhibiting improved yield under abiotic stress conditions (e.g., drought, temperature, or salt stress) in comparison to a control plant grafted onto rootstock that does not transgenically express the miR167.

Transgenic plants expressing a miR167 for use as solanaceous rootstock are made using intraspecific tomato (Solanum lycopersicum) hybrids or interspecific hybrids (usually S. lycopersicum crossed with a wild relative, e.g., S. habrochaites), using transformation methods similar to those for making a transgenic tomato expressing a miR167 as described in Example 1. Tables 1 and 2 provide non-limiting examples of nucleotide sequences of miR167 precursor or hairpin sequences that are expressed in the plants and processed into the corresponding mature miR167 miRNA. The miR167 transgene is generally introgressed into subsequent generations and the resulting stably transgenic plants used as transgenic rootstock for making whole grafted plants (non-transgenic scions grafted onto the transgenic rootstock) having improved traits. The solanaceous rootstock transgenically expressing mirR167 is used for providing grafted tomato plants and grafted eggplant plants; the grafted plants are screened and scion/graft combinations are selected for improved traits, e.g., increased yield or improved fruit quality, when compared to tomato or eggplant plants grafted onto rootstock not expressing miR167. Methods of grafting tomato or eggplant scions onto solanaceous rootstock, and for selecting scion/graft combinations having improved traits such as improved yield, are known in the art. See, e.g., Turhan et al. (2011) Hort. Sci, (Prague), 38:142-149; Liu et al. (2009) Hort. Science, 44:2058-2062. Related art:
Sun et al. 2012 PLoS ONE 7(3): e32017, WO2011/067745, Wu et al. 2006 Development 133:4211-4218.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

What is claimed is:

1. A method of improving abiotic stress tolerance of a plant, the method comprising genetically modifying the plant to express miRNA167 in an abiotic stress responsive manner, wherein a level of expression of total miR167 under said abiotic stress conditions is selected not exceeding 10 fold compared to same in the plant when grown under optimal conditions, thereby improving abiotic stress tolerance of the plant.

2. The method of claim 1, wherein said genetically modifying the plant to express miRNA167 is effected by expressing within the plant an exogenous polynucleotide encoding miR167.

3. The method of claim 2, wherein said exogenous polynucleotide is expressed under an abiotic stress-responsive promoter.

4. The method of claim 3, wherein said abiotic stress-responsive promoter is selected from the group consisting of OsABA2, OsPrx, Wcor413, Lip5, rab16A, XVSAP1 and OsNAC6.

5. The method of claim 3, wherein said abiotic stress-responsive promoter is OsNAC6.

6. The method of claim 1, wherein said level of expression of total miR167 under optimal conditions is as that of miR167 in a non-genetically modified plant of the same species and growth conditions.

7. The method of claim 1, wherein said level of expression of total miR167 under said abiotic stress does not exceed 8 fold as compared to same in the plant when grown under said optimal conditions.

8. The method of claim 1, wherein said level of expression of total miR167 under said abiotic stress does not exceed 5 fold as compared to same in the plant when grown under said optimal conditions.

9. The method of claim 1, wherein said level of expression of total miR167 under said abiotic stress does not exceed 3 fold as compared to same in the plant when grown under said optimal conditions.

10. The method of claim 1, wherein said level of expression of total miR167 under said abiotic stress does not exceed 2 fold as compared to same in the plant when grown under said optimal conditions.

11. The method of claim 1, wherein said level of expression of total miR167 under said abiotic stress does not exceed 1.4-2 fold as compared to same in the plant when grown under said optimal conditions.

12. The method of claim 1, wherein said level of expression of total miR167 under said abiotic stress does not exceed 1.7-2 fold as compared to same in the plant when grown under said optimal conditions.

13. The method of claim 1, further comprising growing the plant under said abiotic stress.

14. The method of claim 1, wherein said abiotic stress is selected from the group consisting of salinity, water deprivation, low temperature, high temperature, heavy metal toxicity, anaerobiosis, nutrient deficiency, nutrient excess, atmospheric pollution and UV irradiation.

15. The method of claim 14, wherein said water deprivation comprises drought.

16. The method of claim 15, wherein said drought is intermittent drought.

17. The method of claim 15, wherein said drought is terminal drought.

18. A plant or a plant cell genetically modified to express miR167, wherein expression of said miRNA167 in the plant cell is abiotic stress responsive and further wherein a level of expression of total miR167 in the plant cell under said abiotic stress does not exceed 10 fold as compared to same in a plant when grown under optimal conditions.

19. The method of claim 13, wherein said abiotic stress is selected from the group consisting of salinity, water deprivation, low temperature, high temperature, heavy metal toxicity, anaerobiosis, nutrient deficiency, nutrient excess, atmospheric pollution and UV irradiation.


SEQUENCE LISTING (738 sequences)

SEQ ID NO: 1 - DNA, 21 bp, Arabidopsis thaliana
tgaagctgccagcatgatcta

SEQ ID NO: 2 - DNA, 138 bp, Arabidopsis thaliana
tggtgcaccggcatctgatgaagctgccagcatgatctaattagctttctttatcctttgttgtgtttcatgacgatggttaagagatcagtctcgattagatcatgttcgcagtttcacccgttgactgtcgcaccc

SEQ ID NO: 3 - DNA, 310 bp, Artificial sequence
  Sequence for cloning into pORE-E2 using Bam HI and KpnI
gatcctgaacagaaaaatctctctttctctttcttgatctgctacggtgaagtctatggtgcaccggcatctgatgaagctgccagcatgatctaattagctttctttatcctttgttgtgtttcatgacgatggttaagagatcagtctcgattagatcatgttcgcagtttcacccgttgactgtcgcacccttctataaaccctaaattttctctctatcttttttagtttgattttcaagacactttgtttctcaatcttcagtctgattttgtgagcttacttctctttctgaggctataggtac

SEQ ID NO: 4 - DNA, 21 bp, Arachis hypogaea
tgaagctgccagcatgatctt

SEQ ID NO: 5 - DNA, 21 bp, Arabidopsis lyrata
tgaagctgccagcatgatcta

SEQ ID NO: 6 - DNA, 21 bp, Arabidopsis lyrata
tgaagctgccagcatgatcta

SEQ ID NO: 7 - DNA, 21 bp, Arabidopsis lyrata
taagctgccagcatgatcttg

SEQ ID NO: 8 - DNA, 22 bp, Arabidopsis lyrata
tgaagctgccagcatgatctgg

SEQ ID NO: 9 - DNA, 21 bp, Aquilegia coerulea
tcaagctgccagcatgatcta

SEQ ID NO: 10 - DNA, 21 bp, Arabidopsis thaliana
tgaagctgccagcatgatcta

SEQ ID NO: 11 - DNA, 21 bp, Arabidopsis thaliana
taagctgccagcatgatcttg

SEQ ID NO: 12 - DNA, 22 bp, Arabidopsis thaliana
tgaagctgccagcatgatctgg

SEQ ID NO: 13 - DNA, 21 bp, Arabidopsis thaliana
tgaagctgccagcatgatctg

SEQ ID NO: 14 - DNA, 21 bp, Brachypodium distachyon
tgaagctgccagcatgatcta

SEQ ID NO: 15 - DNA, 21 bp, Brachypodium distachyon
tgaagctgccagcatgatcta

SEQ ID NO: 16 - DNA, 21 bp, Brachypodium distachyon
tgaagctgccagcatgatcta

SEQ ID NO: 17 - DNA, 22 bp, Brachypodium distachyon
tgaagctgccagcatgatctga

SEQ ID NO: 18 - DNA, 22 bp, Brachypodium distachyon
tgaagctgccagcatgatctga

SEQ ID NO: 19 - DNA, 22 bp, Brassica napus
tgaagctgccagcatgatctaa

SEQ ID NO: 20 - DNA, 22 bp, Brassica napus
tgaagctgccagcatgatctaa

SEQ ID NO: 21 - DNA, 21 bp, Brassica napus
tgaagctgccagcatgatcta

SEQ ID NO: 22 - DNA, 21 bp, Brassica rapa
tgaagctgccagcatgatcta

SEQ ID NO: 23 - DNA, 21 bp, Brassica rapa
tgaagctgccagcatgatcta

SEQ ID NO: 24 - DNA, 21 bp, Brassica rapa
tgaagctgccagcatgatcta

SEQ ID NO: 25 - DNA, 21 bp, Brassica rapa
tgaagctgccagcatgatcta

SEQ ID NO: 26 - DNA, 22 bp, Citrus clementine
tgaagctgccagcatgatctga

SEQ ID NO: 27 - DNA, 22 bp, Citrus clementine
tgaagctgccagcatgatctga

SEQ ID NO: 28 - DNA, 21 bp, Citrus clementina
tgaagctgccagcatgatctg

SEQ ID NO: 29 - DNA, 21 bp, Citrus sinensis
tgaagctgccagcatgatctg

SEQ ID NO: 30 - DNA, 21 bp, Citrus sinensis
tgaagctgccagcatgatctt

SEQ ID NO: 31 - DNA, 21 bp, Citrus sinensis
tgaagctgccagcatgatctg

SEQ ID NO: 32 - DNA, 22 bp, Citrus trifoliata
tgaagctgccagcatgatctga

SEQ ID NO: 33 - DNA, 21 bp, Gossypium hirsutum
tgaagctgccagcatgatcta

SEQ ID NO: 34 - DNA, 21 bp, Glycine max
tgaagctgccagcatgatcta

SEQ ID NO: 35 - DNA, 21 bp, Glycine max
tgaagctgccagcatgatcta

SEQ ID NO: 36 - DNA, 21 bp, Glycine max
tgaagctgccagcatgatctg

SEQ ID NO: 37 - DNA, 21 bp, Glycine max
tgaagctgccagcatgatcta

SEQ ID NO: 38 - DNA, 21 bp, Glycine max
tgaagctgccagcatgatctt

SEQ ID NO: 39 - DNA, 21 bp, Glycine max
tgaagctgccagcatgatctt

SEQ ID NO: 40 - DNA, 22 bp, Glycine max
tgaagctgccagcatgatctga

SEQ ID NO: 41 - DNA, 24 bp, Glycine max
atcatgctggcagcttcaactggt

SEQ ID NO: 42 - DNA, 23 bp, Glycine max
tcatgctggcagcttcaactggt

SEQ ID NO: 43 - DNA, 21 bp, Glycine max
tgaagctgccagcatgatctg

SEQ ID NO: 44 - DNA, 20 bp, Glycine max
tgaagctgccagcatgatct

SEQ ID NO: 45 - DNA, 21 bp, Glycine max
tgaagctgccagcatgatctg

SEQ ID NO: 46 - DNA, 21 bp, Glycine soja
tgaagctgccagcatgatctg

SEQ ID NO: 47 - DNA, 21 bp, Ipomoea nil
tgaagctgccagcatgatctg

SEQ ID NO: 48 - DNA, 21 bp, Lotus japonicus
tgaagctgccagcatgatctg

SEQ ID NO: 49 - DNA, 21 bp, Medicago truncatula
tgaagctgccagcatgatcta

SEQ ID NO: 50 - DNA, 21 bp, Medicago truncatula
tgaagctgccagcatgatctg

SEQ ID NO: 51 - DNA, 21 bp, Oryza sativa
tgaagctgccagcatgatcta

SEQ ID NO: 52 - DNA, 22 bp, Oryza sativa
atcatgcatgacagcctcattt

SEQ ID NO: 53 - DNA, 21 bp, Oryza sativa
tgaagctgccagcatgatcta

SEQ ID NO: 54 - DNA, 21 bp, Oryza sativa
tgaagctgccagcatgatcta

SEQ ID NO: 55 - DNA, 21 bp, Oryza sativa
tgaagctgccagcatgatctg

SEQ ID NO: 56 - DNA, 21 bp, Oryza sativa
tgaagctgccagcatgatctg

SEQ ID NO: 57 - DNA, 21 bp, Oryza sativa
tgaagctgccagcatgatctg

SEQ ID NO: 58 - DNA, 21 bp, Oryza sativa
tgaagctgccagcatgatctg

SEQ ID NO: 59 - DNA, 21 bp, Oryza sativa
tgaagctgccagcatgatctg

SEQ ID NO: 60 - DNA, 21 bp, Oryza sativa
tgaagctgccagcatgatctg

SEQ ID NO: 61 - DNA, 21 bp, Oryza sativa
tgaagctgccagcatgatctg

SEQ ID NO: 62 - DNA, 21 bp, Oryza sativa
tgaagctgccagcatgatctg

SEQ ID NO: 63 - DNA, 21 bp, Oryza sativa
tgaagctgccagcatgatctg

SEQ ID NO: 64 - DNA, 21 bp, Phaseolus coccineus
tgaagctgccagcatgatctt

SEQ ID NO: 65 - DNA, 21 bp, Populus tremula x Populus tremuloides
tgaagctgccagcatgatcta

SEQ ID NO: 66 - DNA, 21 bp, Populus tremula x Populus tremuloides
tgaagctgccagcatgatctg

SEQ ID NO: 67 - DNA, 21 bp, Physcomitrella patens
ggaagctgccagcatgatcct

SEQ ID NO: 68 - DNA, 21 bp, Populus trichocarpa
tgaagctgccagcatgatcta

SEQ ID NO: 69 - DNA, 21 bp, Populus trichocarpa
tgaagctgccagcatgatcta

SEQ ID NO: 70 - DNA, 21 bp, Populus trichocarpa
tgaagctgccagcatgatcta

SEQ ID NO: 71 - DNA, 21 bp, Populus trichocarpa
tgaagctgccagcatgatcta

SEQ ID NO: 72 - DNA, 21 bp, Populus trichocarpa
tgaagctgccagcatgatctg

SEQ ID NO: 73 - DNA, 21 bp, Populus trichocarpa
tgaagctgccagcatgatctt

SEQ ID NO: 74 - DNA, 21 bp, Populus trichocarpa
tgaagctgccagcatgatctt

SEQ ID NO: 75 - DNA, 21 bp, Populus trichocarpa
tgaagctgccaacatgatctg

SEQ ID NO: 76 - DNA, 21 bp, Populus tremuloides
tgaagctgccagcatgatctg

SEQ ID NO: 77 - DNA, 21 bp, Ricinus communis
tgaagctgccagcatgatcta

SEQ ID NO: 78 - DNA, 21 bp, Ricinus communis
tgaagctgccagcatgatcta

SEQ ID NO: 79 - DNA, 22 bp, Ricinus communis
tgaagctgccagcatgatctgg

SEQ ID NO: 80 - DNA, 21 bp, Sorghum bicolor
tgaagctgccagcatgatcta

SEQ ID NO: 81 - DNA, 21 bp, Sorghum bicolor
tgaagctgccagcatgatcta

SEQ ID NO: 82 - DNA, 21 bp, Sorghum bicolor
tgaagctgccagcatgatctg

SEQ ID NO: 83 - DNA, 21 bp, Sorghum bicolor
tgaagctgccagcatgatctg

SEQ ID NO: 84 - DNA, 21 bp, Sorghum bicolor
tgaagctgccagcatgatctg

SEQ ID NO: 85 - DNA, 21 bp, Sorghum bicolor
tgaagctgccagcatgatctg

SEQ ID NO: 86 - DNA, 21 bp, Sorghum bicolor
tgaagctgccagcatgatctg

SEQ ID NO: 87 - DNA, 21 bp, Sorghum bicolor
tgaagctgccagcatgatctg

SEQ ID NO: 88 - DNA, 21 bp, Sorghum bicolor
tgaagctgccagcatgatcta

SEQ ID NO: 89 - DNA, 21 bp, Solanum lycopersicum
tgaagctgccagcatgatcta

SEQ ID NO: 90 - DNA, 21 bp, Saccharum officinarum
tgaagctgccagcatgatctg

SEQ ID NO: 91 - DNA, 21 bp, Saccharum officinarum
tgaagctgccagcatgatctg

SEQ ID NO: 92 - DNA, 21 bp, Saccharum spp
tgaagctgccagcatgatctg

SEQ ID NO: 93 - DNA, 21 bp, Saccharum spp
tgaagctgccagcatgatctg

SEQ ID NO: 94 - DNA, 21 bp, Triticum aestivum
tgaagctgccagcatgatcta

SEQ ID NO: 95 - DNA, 21 bp, Triticum aestivum
tgaagctgacagcatgatcta

SEQ ID NO: 96 - DNA, 21 bp, Theobroma cacao
tgaagctgccagcatgatcta

SEQ ID NO: 97 - DNA, 21 bp, Theobroma cacao
tgaagctgccagcatgatcta

SEQ ID NO: 98 - DNA, 21 bp, Theobroma cacao
tgaagctgccagcatgatctt

SEQ ID NO: 99 - DNA, 21 bp, Vitis vinifera
tgaagctgccagcatgatctg

SEQ ID NO: 100 - DNA, 21 bp, Vitis vinifera
tgaagctgccagcatgatcta

SEQ ID NO: 101 - DNA, 21 bp, Vitis vinifera
tgaagctgccagcatgatctc

SEQ ID NO: 102 - DNA, 21 bp, Vitis vinifera
tgaagctgccagcatgatcta

SEQ ID NO: 103 - DNA, 21 bp, Vitis vinifera
tgaagctgccagcatgatcta

SEQ ID NO: 104 - DNA, 21 bp, Zea mays
tgaagctgccagcatgatcta

SEQ ID NO: 105 - DNA, 22 bp, Zea mays
gatcatgcatgacagcctcatt

SEQ ID NO: 106 - DNA, 21 bp, Zea mays
tgaagctgccagcatgatcta

SEQ ID NO: 107 - DNA, 21 bp, Zea mays
tgaagctgccagcatgatcta

SEQ ID NO: 108 - DNA, 21 bp, Zea mays
tgaagctgccagcatgatcta

SEQ ID NO: 109 - DNA, 23 bp, Zea mays
ggtcatgctgctgcagcctcact

SEQ ID NO: 110 - DNA, 21 bp, Zea mays
tgaagctgccagcatgatctg

SEQ ID NO: 111 - DNA, 22 bp, Zea mays
gatcatgctgtgcagtttcatc

SEQ ID NO: 112 - DNA, 21 bp, Zea mays
tgaagctgccagcatgatctg

SEQ ID NO: 113 - DNA, 21 bp, Zea mays
tgaagctgccagcatgatctg

SEQ ID NO: 114 - DNA, 21 bp, Zea mays
tgaagctgccagcatgatctg

SEQ ID NO: 115 - DNA, 21 bp, Zea mays
tgaagctgccagcatgatctg

SEQ ID NO: 116 - DNA, 21 bp, Zea mays
tgaagctgccagcatgatctg

SEQ ID NO: 117 - DNA, 21 bp, Zea mays
tgaagctgccagcatgatctg

SEQ ID NO: 118 - DNA, 21 bp, Zea mays
tgaagctgccagcatgatctg

SEQ ID NO: 119 - DNA, 21 bp, Zea mays
tgaagctgccagcatgatctg

SEQ ID NO: 120 - DNA, 21 bp, Zea mays
tgaagctgccagcatgatcta

SEQ ID NO: 121 - DNA, 21 bp, Zea mays
tgaagctgccagcatgatcta

SEQ ID NO: 122 - DNA, 21 bp, Zea mays
tgaagctgccagcatgatcta

SEQ ID NO: 123 - DNA, 21 bp, Zea mays
tgaagctgccagcatgatcta

SEQ ID NO: 124 - DNA, 21 bp, Zea mays
tgaagctgccagcatgatcta

SEQ ID NO: 125 - DNA, 21 bp, Zea mays
tgaagctgccagcatgatcta

SEQ ID NO: 126 - DNA, 21 bp, Zea mays
tgaagctgccagcatgatcta

SEQ ID NO: 127 - DNA, 20 bp, Zea mays
tgaagctgccacatgatctg

SEQ ID NO: 128 - DNA, 21 bp, Arachis hypogaea
tgaagctgccagcatgatctt

SEQ ID NO: 129 - DNA, 21 bp, Arabidopsis lyrata
tgaagctgccagcatgatcta

SEQ ID NO: 130 - DNA, 21 bp, Arabidopsis lyrata
tgaagctgccagcatgatcta

SEQ ID NO: 131 - DNA, 21 bp, Arabidopsis lyrata
taagctgccagcatgatcttg

SEQ ID NO: 132 - DNA, 22 bp, Arabidopsis lyrata
tgaagctgccagcatgatctgg

SEQ ID NO: 133 - DNA, 21 bp, Aquilegia coerulea
tcaagctgccagcatgatcta

SEQ ID NO: 134 - DNA, 21 bp, Arabidopsis thaliana
tgaagctgccagcatgatcta

SEQ ID NO: 135 - DNA, 21 bp, Arabidopsis thaliana
tgaagctgccagcatgatcta

SEQ ID NO: 136 - DNA, 22 bp, Arabidopsis thaliana
tgaagctgccagcatgatctgg

SEQ ID NO: 137 - DNA, 21 bp, Arabidopsis thaliana
tgaagctgccagcatgatctg

SEQ ID NO: 138 - DNA, 21 bp, Brachypodium distachyon
tgaagctgccagcatgatcta

SEQ ID NO: 139 - DNA, 21 bp, Brachypodium distachyon
tgaagctgccagcatgatcta

SEQ ID NO: 140 - DNA, 21 bp, Brachypodium distachyon
tgaagctgccagcatgatcta

SEQ ID NO: 141 - DNA, 22 bp, Brachypodium distachyon
tgaagctgccagcatgatctga

SEQ ID NO: 142 - DNA, 22 bp, Brachypodium distachyon
tgaagctgccagcatgatctga

SEQ ID NO: 143 - DNA, 22 bp, Brassica napus
tgaagctgccagcatgatctaa

SEQ ID NO: 144 - DNA, 22 bp, Brassica napus
tgaagctgccagcatgatctaa

SEQ ID NO: 145 - DNA, 21 bp, Brassica napus
tgaagctgccagcatgatcta

SEQ ID NO: 146 - DNA, 21 bp, Brassica rapa
tgaagctgccagcatgatcta

SEQ ID NO: 147 - DNA, 21 bp, Brassica rapa
tgaagctgccagcatgatcta

SEQ ID NO: 148 - DNA, 21 bp, Brassica rapa
tgaagctgccagcatgatcta

SEQ ID NO: 149 - DNA, 21 bp, Brassica rapa
tgaagctgccagcatgatcta

SEQ ID NO: 150 - DNA, 22 bp, Citrus clementine
tgaagctgccagcatgatctga

SEQ ID NO: 151 - DNA, 22 bp, Citrus clementine
tgaagctgccagcatgatctga

SEQ ID NO: 152 - DNA, 21 bp, Citrus clementina
tgaagctgccagcatgatctg

SEQ ID NO: 153 - DNA, 21 bp, Citrus sinensis
tgaagctgccagcatgatctg

SEQ ID NO: 154 - DNA, 21 bp, Citrus sinensis
tgaagctgccagcatgatctt

SEQ ID NO: 155 - DNA, 21 bp, Citrus sinensis
tgaagctgccagcatgatctg

SEQ ID NO: 156 - DNA, 22 bp, Citrus trifoliata
tgaagctgccagcatgatctga

SEQ ID NO: 157 - DNA, 21 bp, Gossypium hirsutum
tgaagctgccagcatgatcta

SEQ ID NO: 158 - DNA, 21 bp, Glycine max
tgaagctgccagcatgatcta

SEQ ID NO: 159 - DNA, 21 bp, Glycine max
tgaagctgccagcatgatcta

SEQ ID NO: 160 - DNA, 21 bp, Glycine max
tgaagctgccagcatgatctg

SEQ ID NO: 161 - DNA, 21 bp, Glycine max
tgaagctgccagcatgatcta

SEQ ID NO: 162 - DNA, 21 bp, Glycine max
tgaagctgccagcatgatctt

SEQ ID NO: 163 - DNA, 21 bp, Glycine max
tgaagctgccagcatgatctt

SEQ ID NO: 164 - DNA, 22 bp, Glycine max
tgaagctgccagcatgatctga

SEQ ID NO: 165 - DNA, 24 bp, Glycine max
atcatgctggcagcttcaactggt

SEQ ID NO: 166 - DNA, 23 bp, Glycine max
tcatgctggcagcttcaactggt

SEQ ID NO: 167 - DNA, 21 bp, Glycine max
tgaagctgccagcatgatctg

SEQ ID NO: 168 - DNA, 20 bp, Glycine max
tgaagctgccagcatgatct

SEQ ID NO: 169 - DNA, 21 bp, Glycine max
tgaagctgccagcatgatctg

SEQ ID NO: 170 - DNA, 21 bp, Glycine soja
tgaagctgccagcatgatctg

SEQ ID NO: 171 - DNA, 21 bp, Ipomoea nil
tgaagctgccagcatgatctg

SEQ ID NO: 172 - DNA, 21 bp, Lotus japonicus
tgaagctgccagcatgatctg

SEQ ID NO: 173 - DNA, 21 bp, Medicago truncatula
tgaagctgccagcatgatcta

SEQ ID NO: 174 - DNA, 21 bp, Medicago truncatula
tgaagctgccagcatgatctg

SEQ ID NO: 175 - DNA, 21 bp, Oryza sativa
tgaagctgccagcatgatcta

SEQ ID NO: 176 - DNA, 22 bp, Oryza sativa
atcatgcatgacagcctcattt

SEQ ID NO: 177 - DNA, 21 bp, Oryza sativa
tgaagctgccagcatgatcta

SEQ ID NO: 178 - DNA, 21 bp, Oryza sativa
tgaagctgccagcatgatcta

SEQ ID NO: 179 - DNA, 21 bp, Oryza sativa
tgaagctgccagcatgatctg

SEQ ID NO: 180 - DNA, 21 bp, Oryza sativa
tgaagctgccagcatgatctg

SEQ ID NO: 181 - DNA, 21 bp, Oryza sativa
tgaagctgccagcatgatctg

SEQ ID NO: 182 - DNA, 21 bp, Oryza sativa
tgaagctgccagcatgatctg

SEQ ID NO: 183 - DNA, 21 bp, Oryza sativa
tgaagctgccagcatgatctg

SEQ ID NO: 184 - DNA, 21 bp, Oryza sativa
tgaagctgccagcatgatctg

SEQ ID NO: 185 - DNA, 21 bp, Oryza sativa
tgaagctgccagcatgatctg

SEQ ID NO: 186 - DNA, 21 bp, Oryza sativa
tgaagctgccagcatgatctg

SEQ ID NO: 187 - DNA, 21 bp, Oryza sativa
tgaagctgccagcatgatctg

SEQ ID NO: 188 - DNA, 21 bp, Phaseolus coccineus
tgaagctgccagcatgatctt

SEQ ID NO: 189 - DNA, 21 bp, Populus tremula x Populus tremuloides
tgaagctgccagcatgatcta

SEQ ID NO: 190 - DNA, 21 bp, Populus tremula x Populus tremuloides
tgaagctgccagcatgatctg

SEQ ID NO: 191 - DNA, 21 bp, Physcomitrella patens
ggaagctgccagcatgatcct

SEQ ID NO: 192 - DNA, 21 bp, Populus trichocarpa
tgaagctgccagcatgatcta

SEQ ID NO: 193 - DNA, 21 bp, Populus trichocarpa
tgaagctgccagcatgatcta

SEQ ID NO: 194 - DNA, 21 bp, Populus trichocarpa
tgaagctgccagcatgatcta

SEQ ID NO: 195 - DNA, 21 bp, Populus trichocarpa
tgaagctgccagcatgatcta

SEQ ID NO: 196 - DNA, 21 bp, Populus trichocarpa
tgaagctgccagcatgatctg

SEQ ID NO: 197 - DNA, 21 bp, Populus trichocarpa
tgaagctgccagcatgatctt

SEQ ID NO: 198 - DNA, 21 bp, Populus trichocarpa
tgaagctgccagcatgatctt

SEQ ID NO: 199 - DNA, 21 bp, Populus trichocarpa
tgaagctgccaacatgatctg

SEQ ID NO: 200 - DNA, 21 bp, Populus tremuloides
tgaagctgccagcatgatctg

SEQ ID NO: 201 - DNA, 21 bp, Ricinus communis
tgaagctgccagcatgatcta

SEQ ID NO: 202 - DNA, 21 bp, Ricinus communis
tgaagctgccagcatgatcta

SEQ ID NO: 203 - DNA, 22 bp, Ricinus communis
tgaagctgccagcatgatctgg

SEQ ID NO: 204 - DNA, 21 bp, Sorghum bicolor
tgaagctgccagcatgatcta

SEQ ID NO: 205 - DNA, 21 bp, Sorghum bicolor
tgaagctgccagcatgatcta

SEQ ID NO: 206 - DNA, 21 bp, Sorghum bicolor
tgaagctgccagcatgatctg

SEQ ID NO: 207 - DNA, 21 bp, Sorghum bicolor
tgaagctgccagcatgatctg

SEQ ID NO: 208 - DNA, 21 bp, Sorghum bicolor
tgaagctgccagcatgatctg

SEQ ID NO: 209 - DNA, 21 bp, Sorghum bicolor
tgaagctgccagcatgatctg

SEQ ID NO: 210 - DNA, 21 bp, Sorghum bicolor
tgaagctgccagcatgatctg

SEQ ID NO: 211 - DNA, 21 bp, Sorghum bicolor
tgaagctgccagcatgatctg

SEQ ID NO: 212 - DNA, 21 bp, Sorghum bicolor
tgaagctgccagcatgatcta

SEQ ID NO: 213 - DNA, 21 bp, Solanum lycopersicum
tgaagctgccagcatgatcta

SEQ ID NO: 214 - DNA, 21 bp, Saccharum officinarum
tgaagctgccagcatgatctg

SEQ ID NO: 215 - DNA, 21 bp, Saccharum officinarum
tgaagctgccagcatgatctg

SEQ ID NO: 216 - DNA, 21 bp, Saccharum spp
tgaagctgccagcatgatctg

SEQ ID NO: 217 - DNA, 21 bp, Saccharum spp
tgaagctgccagcatgatctg

SEQ ID NO: 218 - DNA, 21 bp, Triticum aestivum
tgaagctgccagcatgatcta

SEQ ID NO: 219 - DNA, 21 bp, Triticum aestivum
tgaagctgacagcatgatcta

SEQ ID NO: 220 - DNA, 21 bp, Theobroma cacao
tgaagctgccagcatgatcta

SEQ ID NO: 221 - DNA, 21 bp, Theobroma cacao
tgaagctgccagcatgatcta

SEQ ID NO: 222 - DNA, 21 bp, Theobroma cacao
tgaagctgccagcatgatctt

SEQ ID NO: 223 - DNA, 21 bp, Vitis vinifera
tgaagctgccagcatgatctg

SEQ ID NO: 224 - DNA, 21 bp, Vitis vinifera
tgaagctgccagcatgatcta

SEQ ID NO: 225 - DNA, 21 bp, Vitis vinifera
tgaagctgccagcatgatctc

SEQ ID NO: 226 - DNA, 21 bp, Vitis vinifera
tgaagctgccagcatgatcta

SEQ ID NO: 227 - DNA, 21 bp, Vitis vinifera
tgaagctgccagcatgatcta

SEQ ID NO: 228 - DNA, 21 bp, Zea mays
tgaagctgccagcatgatcta

SEQ ID NO: 229 - DNA, 21 bp, Zea mays
tgaagctgccagcatgatcta

SEQ ID NO: 230 - DNA, 21 bp, Zea mays
tgaagctgccagcatgatcta

SEQ ID NO: 231 - DNA, 21 bp, Zea mays
tgaagctgccagcatgatcta

SEQ ID NO: 232 - DNA, 21 bp, Zea mays
tgaagctgccagcatgatctg

SEQ ID NO: 233 - DNA, 21 bp, Zea mays
tgaagctgccagcatgatctg

SEQ ID NO: 234 - DNA, 21 bp, Zea mays
tgaagctgccagcatgatctg

SEQ ID NO: 235 - DNA, 21 bp, Zea mays
tgaagctgccagcatgatctg

SEQ ID NO: 236 - DNA, 21 bp, Zea mays
tgaagctgccagcatgatctg

SEQ ID NO: 237 - DNA, 21 bp, Zea mays
tgaagctgccagcatgatctg

SEQ ID NO: 238 - DNA, 21 bp, Zea mays
tgaagctgccagcatgatctg

SEQ ID NO: 239 - DNA, 21 bp, Zea mays
tgaagctgccagcatgatctg

SEQ ID NO: 240 - DNA, 21 bp, Zea mays
tgaagctgccagcatgatctg

SEQ ID NO: 241 - DNA, 21 bp, Zea mays
tgaagctgccagcatgatcta

SEQ ID NO: 242 - DNA, 21 bp, Zea mays
tgaagctgccagcatgatcta

SEQ ID NO: 243 - DNA, 21 bp, Zea mays
tgaagctgccagcatgatcta

SEQ ID NO: 244 - DNA, 21 bp, Zea mays
tgaagctgccagcatgatcta

SEQ ID NO: 245 - DNA, 21 bp, Zea mays
tgaagctgccagcatgatcta

SEQ ID NO: 246 - DNA, 21 bp, Zea mays
tgaagctgccagcatgatcta

SEQ ID NO: 247 - DNA, 21 bp, Zea mays
tgaagctgccagcatgatcta

SEQ ID NO: 248 - DNA, 20 bp, Zea mays
tgaagctgccacatgatctg

SEQ ID NO: 249 - DNA, 21 bp, Arachis hypogaea
tgaagctgccagcatgatctt

SEQ ID NO: 250 - DNA, 21 bp, Arabidopsis lyrata
tgaagctgccagcatgatcta

SEQ ID NO: 251 - DNA, 21 bp, Arabidopsis lyrata
tgaagctgccagcatgatcta

SEQ ID NO: 252 - DNA, 21 bp, Arabidopsis lyrata
taagctgccagcatgatcttg

SEQ ID NO: 253 - DNA, 22 bp, Arabidopsis lyrata
tgaagctgccagcatgatctgg

SEQ ID NO: 254 - DNA, 21 bp, Aquilegia coerulea
tcaagctgccagcatgatcta

SEQ ID NO: 255 - DNA, 21 bp, Arabidopsis thaliana
tgaagctgccagcatgatcta

SEQ ID NO: 256 - DNA, 21 bp, Arabidopsis thaliana
tgaagctgccagcatgatcta

SEQ ID NO: 257 - DNA, 21 bp, Arabidopsis thaliana
taagctgccagcatgatcttg

SEQ ID NO: 258 - DNA, 21 bp, Arabidopsis thaliana
tgaagctgccagcatgatctg

SEQ ID NO: 259 - DNA, 21 bp, Brachypodium distachyon
tgaagctgccagcatgatcta

SEQ ID NO: 260 - DNA, 21 bp, Brachypodium distachyon
tgaagctgccagcatgatcta

SEQ ID NO: 261 - DNA, 21 bp, Brachypodium distachyon
tgaagctgccagcatgatcta

SEQ ID NO: 262 - DNA, 22 bp, Brachypodium distachyon
tgaagctgccagcatgatctga

SEQ ID NO: 263 - DNA, 22 bp, Brachypodium distachyon
tgaagctgccagcatgatctga

SEQ ID NO: 264 - DNA, 22 bp, Brassica napus
tgaagctgccagcatgatctaa

SEQ ID NO: 265 - DNA, 22 bp, Brassica napus
tgaagctgccagcatgatctaa

SEQ ID NO: 266 - DNA, 21 bp, Brassica napus
tgaagctgccagcatgatcta

SEQ ID NO: 267 - DNA, 21 bp, Brassica rapa
tgaagctgccagcatgatcta

SEQ ID NO: 268 - DNA, 21 bp, Brassica rapa
tgaagctgccagcatgatcta

SEQ ID NO: 269 - DNA, 21 bp, Brassica rapa
tgaagctgccagcatgatcta

SEQ ID NO: 270 - DNA, 21 bp, Brassica rapa
tgaagctgccagcatgatcta

SEQ ID NO: 271 - DNA, 22 bp, Citrus clementine
tgaagctgccagcatgatctga

SEQ ID NO: 272 - DNA, 22 bp, Citrus clementine
tgaagctgccagcatgatctga

SEQ ID NO: 273 - DNA, 21 bp, Citrus clementina
tgaagctgccagcatgatctg

SEQ ID NO: 274 - DNA, 21 bp, Citrus sinensis
tgaagctgccagcatgatctg

SEQ ID NO: 275 - DNA, 21 bp, Citrus sinensis
tgaagctgccagcatgatctt

SEQ ID NO: 276 - DNA, 21 bp, Citrus sinensis
tgaagctgccagcatgatctg

SEQ ID NO: 277 - DNA, 22 bp, Citrus trifoliata
tgaagctgccagcatgatctga

SEQ ID NO: 278 - DNA, 21 bp, Gossypium hirsutum
tgaagctgccagcatgatcta

SEQ ID NO: 279 - DNA, 21 bp, Glycine max
tgaagctgccagcatgatcta

SEQ ID NO: 280 - DNA, 21 bp, Glycine max
tgaagctgccagcatgatcta

SEQ ID NO: 281 - DNA, 21 bp, Glycine max
tgaagctgccagcatgatctg

SEQ ID NO: 282 - DNA, 21 bp, Glycine max
tgaagctgccagcatgatcta

SEQ ID NO: 283 - DNA, 21 bp, Glycine max
tgaagctgccagcatgatctt

SEQ ID NO: 284 - DNA, 21 bp, Glycine max
tgaagctgccagcatgatctt

SEQ ID NO: 285 - DNA, 22 bp, Glycine max
tgaagctgccagcatgatctga

SEQ ID NO: 286 - DNA, 24 bp, Glycine max
atcatgctggcagcttcaactggt

SEQ ID NO: 287 - DNA, 23 bp, Glycine max
tcatgctggcagcttcaactggt

SEQ ID NO: 288 - DNA, 21 bp, Glycine max
tgaagctgccagcatgatctg

SEQ ID NO: 289 - DNA, 20 bp, Glycine max
tgaagctgccagcatgatct

SEQ ID NO: 290 - DNA, 21 bp, Glycine max
tgaagctgccagcatgatctg

SEQ ID NO: 291 - DNA, 21 bp, Glycine soja
tgaagctgccagcatgatctg

SEQ ID NO: 292 - DNA, 21 bp, Ipomoea nil
tgaagctgccagcatgatctg

SEQ ID NO: 293 - DNA, 21 bp, Lotus japonicus
tgaagctgccagcatgatctg

SEQ ID NO: 294 - DNA, 21 bp, Medicago truncatula
tgaagctgccagcatgatcta

SEQ ID NO: 295 - DNA, 21 bp, Medicago truncatula
tgaagctgccagcatgatctg

SEQ ID NO: 296 - DNA, 21 bp, Oryza sativa
tgaagctgccagcatgatcta

SEQ ID NO: 297 - DNA, 22 bp, Oryza sativa
atcatgcatgacagcctcattt

SEQ ID NO: 298 - DNA, 21 bp, Oryza sativa
tgaagctgccagcatgatcta

SEQ ID NO: 299 - DNA, 21 bp, Oryza sativa
tgaagctgccagcatgatcta

SEQ ID NO: 300 - DNA, 21 bp, Oryza sativa
tgaagctgccagcatgatctg

SEQ ID NO: 301 - DNA, 21 bp, Oryza sativa
tgaagctgccagcatgatctg

SEQ ID NO: 302 - DNA, 21 bp, Oryza sativa
tgaagctgccagcatgatctg

SEQ ID NO: 303 - DNA, 21 bp, Oryza sativa
tgaagctgccagcatgatctg

SEQ ID NO: 304 - DNA, 21 bp, Oryza sativa
tgaagctgccagcatgatctg

SEQ ID NO: 305 - DNA, 21 bp, Oryza sativa
tgaagctgccagcatgatctg

SEQ ID NO: 306 - DNA, 21 bp, Oryza sativa
tgaagctgccagcatgatctg

SEQ ID NO: 307 - DNA, 21 bp, Oryza sativa
tgaagctgccagcatgatctg

SEQ ID NO: 308 - DNA, 21 bp, Oryza sativa
tgaagctgccagcatgatctg

SEQ ID NO: 309 - DNA, 21 bp, Phaseolus coccineus
tgaagctgccagcatgatctt

SEQ ID NO: 310 - DNA, 21 bp, Populus tremula x Populus tremuloides
tgaagctgccagcatgatcta

SEQ ID NO: 311 - DNA, 21 bp, Populus tremula x Populus tremuloides
tgaagctgccagcatgatctg

SEQ ID NO: 312 - DNA, 21 bp, Physcomitrella patens
ggaagctgccagcatgatcct

SEQ ID NO: 313 - DNA, 21 bp, Populus trichocarpa
tgaagctgccagcatgatcta

SEQ ID NO: 314 - DNA, 21 bp, Populus trichocarpa
tgaagctgccagcatgatcta

SEQ ID NO: 315 - DNA, 21 bp, Populus trichocarpa
tgaagctgccagcatgatcta

SEQ ID NO: 316 - DNA, 21 bp, Populus trichocarpa
tgaagctgccagcatgatcta

SEQ ID NO: 317 - DNA, 21 bp, Populus trichocarpa
tgaagctgccagcatgatctg

SEQ ID NO: 318 - DNA, 21 bp, Populus trichocarpa
tgaagctgccagcatgatctt

SEQ ID NO: 319 - DNA, 21 bp, Populus trichocarpa
tgaagctgccagcatgatctt

SEQ ID NO: 320 - DNA, 21 bp, Populus trichocarpa
tgaagctgccaacatgatctg

SEQ ID NO: 321 - DNA, 21 bp, Populus tremuloides
tgaagctgccagcatgatctg

SEQ ID NO: 322 - DNA, 21 bp, Ricinus communis
tgaagctgccagcatgatcta

SEQ ID NO: 323 - DNA, 21 bp, Ricinus communis
tgaagctgccagcatgatcta

SEQ ID NO: 324 - DNA, 22 bp, Ricinus communis
tgaagctgccagcatgatctgg

SEQ ID NO: 325 - DNA, 21 bp, Sorghum bicolor
tgaagctgccagcatgatcta

SEQ ID NO: 326 - DNA, 21 bp, Sorghum bicolor
tgaagctgccagcatgatcta

SEQ ID NO: 327 - DNA, 21 bp, Sorghum bicolor
tgaagctgccagcatgatctg

SEQ ID NO: 328 - DNA, 21 bp, Sorghum bicolor
tgaagctgccagcatgatctg

SEQ ID NO: 329 - DNA, 21 bp, Sorghum bicolor
tgaagctgccagcatgatctg

SEQ ID NO: 330 - DNA, 21 bp, Sorghum bicolor
tgaagctgccagcatgatctg

SEQ ID NO: 331 - DNA, 21 bp, Sorghum bicolor
tgaagctgccagcatgatctg

SEQ ID NO: 332 - DNA, 21 bp, Sorghum bicolor
tgaagctgccagcatgatctg

SEQ ID NO: 333 - DNA, 21 bp, Sorghum bicolor
tgaagctgccagcatgatcta

SEQ ID NO: 334 - DNA, 21 bp, Solanum lycopersicum
tgaagctgccagcatgatcta

SEQ ID NO: 335 - DNA, 21 bp, Saccharum officinarum
tgaagctgccagcatgatctg

SEQ ID NO: 336 - DNA, 21 bp, Saccharum officinarum
tgaagctgccagcatgatctg

SEQ ID NO: 337 - DNA, 21 bp, Saccharum spp
tgaagctgccagcatgatctg

SEQ ID NO: 338 - DNA, 21 bp, Saccharum spp
tgaagctgccagcatgatctg

SEQ ID NO: 339 - DNA, 21 bp, Triticum aestivum
tgaagctgccagcatgatcta

SEQ ID NO: 340 - DNA, 21 bp, Triticum aestivum
tgaagctgacagcatgatcta

SEQ ID NO: 341 - DNA, 21 bp, Theobroma cacao
tgaagctgccagcatgatcta

SEQ ID NO: 342 - DNA, 21 bp, Theobroma cacao
tgaagctgccagcatgatcta

SEQ ID NO: 343 - DNA, 21 bp, Theobroma cacao
tgaagctgccagcatgatctt

SEQ ID NO: 344 - DNA, 21 bp, Vitis vinifera
tgaagctgccagcatgatctg

SEQ ID NO: 345 - DNA, 21 bp, Vitis vinifera
tgaagctgccagcatgatcta

SEQ ID NO: 346 - DNA, 21 bp, Vitis vinifera
tgaagctgccagcatgatctc

SEQ ID NO: 347 - DNA, 21 bp, Vitis vinifera
tgaagctgccagcatgatcta

SEQ ID NO: 348 - DNA, 21 bp, Vitis vinifera
tgaagctgccagcatgatcta

SEQ ID NO: 349 - DNA, 21 bp, Zea mays
tgaagctgccagcatgatcta

SEQ ID NO: 350 - DNA, 21 bp, Zea mays
tgaagctgccagcatgatcta

SEQ ID NO: 351 - DNA, 21 bp, Zea mays
tgaagctgccagcatgatcta

SEQ ID NO: 352 - DNA, 21 bp, Zea mays
tgaagctgccagcatgatcta

SEQ ID NO: 353 - DNA, 21 bp, Zea mays
tgaagctgccagcatgatctg

SEQ ID NO: 354 - DNA, 21 bp, Zea mays
tgaagctgccagcatgatctg

SEQ ID NO: 355 - DNA, 21 bp, Zea mays
tgaagctgccagcatgatctg

SEQ ID NO: 356 - DNA, 21 bp, Zea mays
tgaagctgccagcatgatctg

SEQ ID NO: 357 - DNA, 21 bp, Zea mays
tgaagctgccagcatgatctg

SEQ ID NO: 358 - DNA, 21 bp, Zea mays
tgaagctgccagcatgatctg

SEQ ID NO: 359 - DNA, 21 bp, Zea mays
tgaagctgccagcatgatctg

SEQ ID NO: 360 - DNA, 21 bp, Zea mays
tgaagctgccagcatgatctg

SEQ ID NO: 361 - DNA, 21 bp, Zea mays
tgaagctgccagcatgatctg

SEQ ID NO: 362 - DNA, 21 bp, Zea mays
tgaagctgccagcatgatcta

SEQ ID NO: 363 - DNA, 21 bp, Zea mays
tgaagctgccagcatgatcta

SEQ ID NO: 364 - DNA, 21 bp, Zea mays
tgaagctgccagcatgatcta

SEQ ID NO: 365 - DNA, 21 bp, Zea mays
tgaagctgccagcatgatcta

SEQ ID NO: 366 - DNA, 21 bp, Zea mays
tgaagctgccagcatgatcta

SEQ ID NO: 367 - DNA, 21 bp, Zea mays
tgaagctgccagcatgatcta

SEQ ID NO: 368 - DNA, 21 bp, Zea mays
tgaagctgccagcatgatcta

SEQ ID NO: 369 - DNA, 20 bp, Zea mays
tgaagctgccacatgatctg

SEQ ID NO: 370 - DNA, 118 bp, Arachis hypogaea
gatcatgcaccactacaagttgaagctgccagcatgatcttaactttccctctcctatgatttgttggggtgagatcagatcatgtggcagtttcacctagttgttggaagcatgaat

SEQ ID NO: 371 - DNA, 138 bp, Arabidopsis lyrata
ggtgcaccggcatctgatgaagctgccagcatgatctaattagctttctttatatctgttgttgtgtttcataacgatggttaagagatgagtctcgattagatcatgttcgcagtttcacccgttgactgtcgcacc

SEQ ID NO: 372 - DNA, 195 bp, Arabidopsis lyrata
atctgcacaacttgttgctcaggtattttgaagacaagtccacaagggaacaagtgaagctgccagcatgatctatctttggttaagagatgaatgtgtaaacatattgcttaaacccaagctaggtcatgctctgacagcctcactccttcctggtttaggaccattcactgataaagcattccacatgccgat

SEQ ID NO: 373 - DNA, 159 bp, Arabidopsis lyrata
cagtagcagttaagctgccagcatgatcttgtcttcctctcttaagtttcatatataatcaagttaatataaagattttgtacaattcttgttcttattatatgatcatagcttagagagagagagactaggtcatgctggtagtttcacctgctaatg

SEQ ID NO: 374 - DNA, 327 bp, Arabidopsis lyrata
gatctatatctatgctggtttttagaggctgaagctgccagcatgatctggtaattgctacatacgacatacacacatatactagttaatttccacacctataaaagtttttttcctacaacttaaagcttttttccttcctctttttaataattagtgatctctagttctttgcctacttgtaatatatatttacggtggattcatgcatgtgtgtatatatatacatagtttacatgcatgcattttgtgtatgtgtgtgtgtatagatagtagtactaggtcatcctgcagcttcagtcactaaatcaccaacaatatcaaatc

SEQ ID NO: 375 - DNA, 68 bp, Aquilegia coerulea
tcaagctgccagcatgatctaaaaatctctgcatgtggggattatcagatcatgctgcagtttaacct

SEQ ID NO: 376 - DNA, 109 bp, Arabidopsis thaliana
gggaacaagtgaagctgccagcatgatctatctttggttaagagatgaatgtggaaacatattgcttaaacccaagctaggtcatgctctgacagcctcactccttcct

SEQ ID NO: 377 - DNA, 160 bp, Arabidopsis thaliana
ccagtagcagttaagctgccagcatgatcttgtcttcctctcttaggtttcatatatagttaataaatattttatatatttcttgttcttacaagattatatgatcatagcttagagagagagagagactaggtcatgctggtagtttcacctgctaatg

SEQ ID NO: 378 - DNA, 377 bp, Arabidopsis thaliana
tgttggtttttagaagctgaagctgccagcatgatctggtaatcgctacatacgacatacacacatcactaaacttctttataatttatgcacacacatacagctcttaatggccacaactcaaagttataattagtgcatgatctctagttatttgactgcttttaatatatgtttatggattcacgcatgtgtgtgtatgtacataatttacatgcatgcactttgtgtatggtacacatcaatttgaacccgttcaaaattctgtttttattagtatatatatagatgtatgtggtgtgtgtgtcagtgtgtgtgtgtgtttatagatagtagtactaggtcatcctgcagcttcagtcactaaatcaccaaca

SEQ ID NO: 379 - DNA, 342 bp, Arabidopsis thaliana
tgaagctgccagcatgatctggtaatcgctacatacgacatacacacatcactaaacttctttataatttatgcacacacatacagctcttaatggccacaactcaaagttataattagtgcatgatctctagttatttgactgcttttaatatatgtttatggattcacgcatgtgtgtgtatgtacataatttacatgcatgcactttgtgtatggtacacatcaatttgaacccgttcaaaattctgtttttattagtatatatatagatgtatgtggtgtgtgtgtcagtgtgtgtgtgtgtttatagatagtagtactaggtcatcctgcagcttca

SEQ ID NO: 380 - DNA, 91 bp, Brachypodium distachyon
agagaaagcgtgaagctgccagcatgatctatctgacttgtggtggcaagtgccacagcaaattcagcaacgccgtggcttcggggccggc

SEQ ID NO: 381 - DNA, 91 bp, Brachypodium distachyon; identical to SEQ ID NO: 380

SEQ ID NO: 382 - DNA, 190 bp, Brachypodium distachyon
gtgctacttacttactgcccgagggaacgagtgaagctgccagcatgatctagctcagcgtgatcaagcaagattcacacatacacgtgtggtttttttgagctatagctcgattgatcttgaggtcatgccttgctaggtcatgctgcggcagcctcacttcttcccgccgtttgggcatgcacagctg

SEQ ID NO: 383 - DNA, 159 bp, Brachypodium distachyon
ttcacttgctgtggtgcatcttctaggagctgaagctgccagcatgatctgacgagagttcctcgtctgatagcaatgtttaattctcttgtcatgactaatgatcagatcatgctgtgcagtttcatctgcttgtggatgcacaagatactgttcata

SEQ ID NO: 384 - DNA, 204 bp, Brachypodium distachyon
tggacggctcaatttgatggtgtgagaggttgaagctgccagcatgatctgatcaccgtccaacgtaaccgaacacatgtcgatcgacttccgattgcgccggttatcttggtaggaatatatatatatgagcttccattgcaagggttcttcagatcatgttgcagcttcactctctcatcaccaccgaaagatccaaattaa

SEQ ID NO: 385 - DNA, 132 bp, Brassica napus
ggtgtacaggcatctgatgaagctgccagcatgatctaattaactttctttctctgttgattttatgacaatggaaaagagatgagtgtcgattagatcatgttcgcagtttcacccattgactgtcgcacc

SEQ ID NO: 386 - DNA, 124 bp, Brassica napus
ggcgcaccggcatctgatgaagctgccagcatgatctaattatctttctttctctgttgacgatggaaaagacatgagtgttgattagatcatgttcgcagtttcacccgttgactgtctcgcc

SEQ ID NO: 387 - DNA, 134 bp, Brassica napus
ggtgcaccggcatctgatgaagctgccagcatgatctagttaactttatttctccgttgtttatccatgacaatggaaaagggataagtgtcgattagatcatgttcgtagtttcacccgttgactgtcgcatc

SEQ ID NO: 388 - DNA, 134 bp, Brassica rapa; identical to SEQ ID NO: 387

SEQ ID NO: 389 - DNA, 132 bp, Brassica rapa; identical to SEQ ID NO: 385

SEQ ID NO: 390 - DNA, 132 bp, Brassica rapa; identical to SEQ ID NO: 385

SEQ ID NO: 391 - DNA, 124 bp, Brassica rapa; identical to SEQ ID NO: 386

SEQ ID NO: 392 - DNA, 121 bp, Citrus clementine
catattcgtgcactagtagtagttgaagctgccagcatgatctgaactttccttgacctccatctctagggaaaggccagatcatctggcagtttcacctattgatggtagcatggccaga

SEQ ID NO: 393 - DNA, 202 bp, Citrus clementine
attcgtgcactagtagtagttgaagctgccagcatgatctgaactttccttgacctccatctctagggaaaggccagatcatctggcagtttcacctattgatggtagcatggccagaaaccctaatttcttctcctccaccagatcgttctcaacaaacccagtaggttttggcagatgaaaaaccctagaaacaggtatc

SEQ ID NO: 394 - DNA, 96 bp, Citrus clementina
tagtagtagttgaagctgccagcatgatctgaactttccttgacctccatctctagggaaaggccagatcatctggcagtttcacctattgatggt

SEQ ID NO: 395 - DNA, 103 bp, Citrus sinensis
gcactagtagtagttgaagctgccagcatgatctgaactttccttgacctccatctctagggaaaggccagatcatctggcagtttcacctattgatggtagc

SEQ ID NO: 396 - DNA, 123 bp, Citrus sinensis
atcgggcaccactatcagatgaagctgccagcatgatcttaactttcctcctttgctcgaggaatgatacagatcatgcggcagtttcacctgttcgttggttgcacgaaattacgagtccag

SEQ ID NO: 397 - DNA, 341 bp, Citrus sinensis
  misc_feature (269)..(269): n is a, c, g, or t
tttgagagattgaagctgccagcatgatctggtaatcaacctttttgtatatatatatatatattaattccttatagtttttagatttaatttcttttaattagatccatggtttcaattctattgaataaatggtggggttttatattttcgtgcaattattaagaggatagatggaatagcgcctttaaatccaatcacttttttagttttattttgatcttttttgccccctaaaattaagggtaaaggttaatatgtgagagagntttagggtgtgatttattagcttcgtagatgaatggttccatcaggtcatcttgcagcttcaattactcatt

SEQ ID NO: 398 - DNA, 121 bp, Citrus trifoliata; identical to SEQ ID NO: 392

SEQ ID NO: 399 - DNA, 105 bp, Gossypium hirsutum
gggaaaaagtgaagctgccagcatgatctatcttccgttagtaagatgcggatgctatattgctaaccctagctaggtcatgctgcgacagcctcactccttcct

SEQ ID NO: 400 - DNA, 119 bp, Glycine max
gaagttcgcaaaggaaaaagtgaagctgccagcatgatctacctttggttagagagctcaagagtgctaaccctgactaggtcatgctgtgacagcctcactccttcctatttggggac

SEQ ID NO: 401 - DNA, 121 bp, Glycine max
aagggtcacaaaggaaaaagtgaagctgccagcatgatctagctttggttagtgggagcgagagagtgctaaccctcactaggtcatgctgtgctagcctcactccttcctatttggagac

SEQ ID NO: 402 - DNA, 375 bp, Glycine max
tttgagaggttgaagctgccagcatgatctggtaaatcacatacttttttttttctcacctctcatgcctaatttttaagcaccagtcattagagaaaataatggtgaaaaatccatctattcaattttttttttcaaattcaaggtttccagtatgtatcactaatggtgaaaaaagtgatggaattttgtagaacatgggttaaatttactttttttttttttgagttttcattttcttcaagtttctgagccaagaaataaaagagacttataaattggaattaatacttaaaggaaacccaccagaagggcaatttggttatcataagatgtggtttccatcaggtcatcttgcagcttcaatcactcaat

SEQ ID NO: 403 - DNA, 121 bp, Glycine max
aagggtcacaagggaaaaagtgaagctgccagcatgatctagctttggttagtgggagccagagagtgctaaccctcactaggtcatgctgtgctagcctcactccttcctatttggagac

SEQ ID NO: 404 - DNA, 109 bp, Glycine max
tcatgcaccactaccagttgaagctgccagcatgatcttaacttccctcacttgccgtggaaagatcagatcatgtggcagtttcacctagtagttgctggccgcatga

SEQ ID NO: 405 - DNA, 109 bp, Glycine max
tcatgcaccactaccagttgaagctgccagcatgatcttaacttccctcacttgctgtggaaagatcagatcatgtggcagtttcacctagtagttgttggccgcatga

SEQ ID NO: 406 - DNA, 78 bp, Glycine max
cagcagttgaagctgccagcatgatctgagtttaccttctattggtaagaacagatcatgtggctgcttcacctgttg

SEQ ID NO: 407 - DNA, 151 bp, Glycine max
aactactaggtgaaactgccacatgatctgatctttccacagcaagtgagggaagttaagatcatgctggcagcttcaactggtagtggtgcatgatggtagacagatattgggaagaacaagaacaagtgttctaaaaggtgatgatgta

SEQ ID NO: 408 - DNA, 151 bp, Glycine max
caactactaggtgaaactgccacatgatctgatctttccacggcaagtgagggaagttaagatcatgctggcagcttcaactggtagtggtgcatgatggtagacagatattgggaagaacaagaaccagaacaagtgttctaaaaggtaa

SEQ ID NO: 409 - DNA, 78 bp, Glycine max; identical to SEQ ID NO: 406

SEQ ID NO: 410 - DNA, 64 bp, Glycine max
tgaagctgccagcatgatctgagtttaccttctattggtaagaacagatcatgtggctgcttca

SEQ ID NO: 411 - DNA, 109 bp, Glycine max
caagatgttgttgttggtaccctctcacaggatttgcttcaatgaaaggggttcatcactcttttcatcacatgttggtttgagaggttgaagctgccagcatgatctg

SEQ ID NO: 412 - DNA, 80 bp, Glycine soja
gcagcagttgaagctgccagcatgatctgagtttaccttctattggtaagaacagatcatgtggctgcttcacctgttga

SEQ ID NO: 413 - DNA, 271 bp, Ipomoea nil
tgaagctgccagcatgatctggtaagatagaacaaaatcttgggttttctttttcccactttttcttttatggggttttcatctttctgcagaaatagaattcactgtaccaaaagaacacatctttggggtttttttctgttcttcattctccccccttctgtttcaattcttttttttggttggttggtatgggttctgtacatagtttaaagattggagagtgaattatgcctaaagtagacagatctcttgtgcgcaccggtattta

SEQ ID NO: 414 - DNA, 108 bp, Lotus japonicus
gttcgtgcacctgcaatagttgaagctgccagcatgatctgagcttaccttcttgtaataatggtaagaacagatcatatggcagcttcacctgttgaatggaagcat

SEQ ID NO: 415 - DNA, 320 bp, Medicago truncatula
aaaagtgaagctgccagcatgatctaggtttggttatacaatagtagtattgagaaggaactatatacgttttttttttactataccacaaaaaaagattactctctttcacaaaataggtattaaagtgccatgatttttgcattactaatgggaaaataaattttggacaccgaatttctcactttttttttatatagataggaaataggttttggtggtatttttttgtggtacagtaaaaaatagccgctatatccatacaagtagtactgctagcataaccctgactaggtcatgctgtgctagcctcactcctt

SEQ ID NO: 416 - DNA, 207 bp, Medicago truncatula
caatgacagttgaagctgccagcatgatctgtgctttccttcctgtgtatatactttaatttccagctgaatttaaatataaccaaaaaaataaatatgtttggtctaaattttgatcaaacttatatatatttttgcttatgtttaagtctggggtgagtttatttgtggtaagaacagatcatgttggagcttcacctgttaaat

SEQ ID NO: 417 - DNA, 141 bp, Oryza sativa
tagtgtgaatgagtgaagctgccagcatgatctagctctgattaatcggcactgttggcgtacagtcgattgactaatcgtcagatctgtgtgtgtaaatcactgttagatcatgcatgacagcctcatttcttcacactg

SEQ ID NO: 418 - DNA, 141 bp, Oryza sativa; identical to SEQ ID NO: 417

SEQ ID NO: 419 - DNA, 163 bp, Oryza sativa
gtgcccaagagaaagcgtgaagctgccagcatgatctaacttgcagacaagaaatcagctcagctcgctggtttcgaacaggaaggcggctagctgaggcttcttctgagtacgtgatggttagatcatgctgtgacagtttcactccttccctgttgggcac

SEQ ID NO: 420 - DNA, 163 bp, Oryza sativa
tgtccaagggaacgagtgaagctgccagcatgatctagctctgaatgatcaacaagatgtgctcccacactgccttcctgtggatcttgagctgttgctagtcttgtggtcatgccttgctaggtcatgctgcggcagcctcacttcttcccattgttgggca

SEQ ID NO: 421 - DNA, 110 bp, Oryza sativa
cattaggagctgaagctgccagcatgatctgatgagtgcttattaggtgagggcagaattgactgccaaaacaaagatcagatcatgctgtgcagtttcatctgcttgtg

SEQ ID NO: 422 - DNA, 273 bp, Oryza sativa
tgtgagagaatgaagctgccagcatgatctggttgtcaggcatgagccaaatctatccatggtgttggtggtactgaaattaccgcgttttcgaggtttttcgtcgtgtcaacttgcgaagggaattacgggttcttgatgagcattggtgataggaggtgtgggcttggttagtagaggtagaattatgattgttcttgtgagtttcagtaagaggtgggagtgattggaatttggctccatcagatcatgttgcagcttcactctctcacc

SEQ ID NO: 423 - DNA, 113 bp, Oryza sativa
cacaagtggatgaagctgccagcatgatctgatcacagtagttctctagctgatgatgatttacaaaacctagagacatgcatcagatcatctggcagtttcatcttctcatg

SEQ ID NO: 424 - DNA, 82 bp, Oryza sativa
cataagcaggtgaagctgccagcatgatctgaaagcatctcaaaccagcgatcagatcatccggcagcttcatcttctcatg

SEQ ID NO: 425 - DNA, 120 bp, Oryza sativa
cacaagttggtgaagctgccagcatgatctgatgatgatgatgatccacctctctcatctgtgttcttgattaattacggatcaatcgatcaggtcatgctgtagtttcatctgctggtt

SEQ ID NO: 426 - DNA, 201 bp, Oryza sativa
tgtgagaggctgaagctgccagcatgatctggtccatgagttgcactgctgaatatattgaattcagccaggagctgctactgcagttctgatctcgatctgcattcgttgttctgagctatgtatggatttgatcggtttgaaggcatccatgtctttaatttcatcgatcagatcatgttgcagcttcactctctcact

SEQ ID NO: 427 - DNA, 160 bp, Oryza sativa
ttgtgatgtgtgcaccttaagcagctgaagctgccagcatgatctgatcttttgcgatctctttttttatctgaataagttgatggaaatattgggttcctaagattcagatcgtgctgcgcagtttcatctgctaatcgatgcactacactgtgaattt

SEQ ID NO: 428 - DNA, 100 bp, Oryza sativa
tgaagctgccagcatgatctgatgatgatgatgatccacctctctcatctgtgttcttgattaattacggatcaatcgatcaggtcatgctgtagtttca

SEQ ID NO: 429 - DNA, 90 bp, Oryza sativa
tgaagctgccagcatgatctgatgagtgcttattaggtgagggcagaattgactgccaaaacaaagatcagatcatgctgtgcagtttca

SEQ ID NO: 430 - DNA, 69 bp, Phaseolus coccineus
tgaagctgccagcatgatcttaacttccctcacttggttgaggagagatcagatcatgtggcagtttca

SEQ ID NO: 431 - DNA, 108 bp, Populus tremula x Populus tremuloides
agggaaaaggtgaagctgccagcatgatctatctttggttagagaagtatagaagcgaagaactaaccctagctaggtcatgctctgacagcctcactccttcctgtt

SEQ ID NO: 432 - DNA, 431 bp, Populus tremula x Populus tremuloides
  misc_feature (327)..(327): n is a, c, g, or t
tttgagaggttgaagctgccagcatgatctggtaatgaacgtgttactctcatttatatatatattaacattaatcttatagcagtatctgtaggtagaaaaaatttaatgttgtcaaagatatatactgaatcgtggttgctaggtttgtattactagtttaggatgcatgtttttgatcttatgatgatcaattgcttgtgagttcctaggcaatgaaaacagaatatatactggtgatttttcccagtaaaattgtcgagaaaagggaattgcactaatagggaagacgcataggtaaacttgtatctaaatggtatatgtatnttccaggcaaaagggtagaaacctaatnagaaactagcttgaactcagagctataaaagtatatggttccatcaggtcatctagcagcttcaatcactcactca

SEQ ID NO: 433 - DNA, 82 bp, Physcomitrella patens
accaaaagttggaagctgccagcatgatcctttaacttttctagagggaaagatcagatcatctggctgctttcatcctgtt

SEQ ID NO: 434 - DNA, 89 bp, Populus trichocarpa
cactagcagttgaagctgccagcatgatctaacttccttgcttctttatcaaggatggatttagatcatgtggtggtttcacctgttga

SEQ ID NO: 435 - DNA, 96 bp, Populus trichocarpa
agggaaaaagtgaagctgccagcatgatctatctttggttagagaaagaaaggactaaccctagctaggtcatgctgtgacagcctcactccttcc

SEQ ID NO: 436 - DNA, 89 bp, Populus trichocarpa
cactagcagttgaagctgccagcatgatctaaattaacctccttctttatcaaggatggattagatcatgtggtagtttcacctgctga

SEQ ID NO: 437 - DNA, 105 bp, Populus trichocarpa
agggaaaaggtgaagctgccagcatgatctatctttggttagagaaggatagaagcgaaagaactaaccctagctaggtcatgctctgacagcctcactccttcc

SEQ ID NO: 438 - DNA, 91 bp, Populus trichocarpa
cactagtagttgaagctgccagcatgatctgaactttccttaattttcctatacgggaaagactagatcatgtggtagtttcatctattga

SEQ ID NO: 439 - DNA, 87 bp, Populus trichocarpa
ctctatcagttgaagctgccagcatgatcttagccttcctcctttgttgaggaaagaaacagatcatgtggcagtttcacctgttgt

SEQ ID NO: 440 - DNA, 86 bp, Populus trichocarpa
cactatcagttgaagctgccagcatgatcttaacctccctcctttgtcgaggaaagaacagatcatgtggcagtttcacctgaagt

SEQ ID NO: 441 - DNA, 91 bp, Populus trichocarpa
cgctattagttgaagctgccaacatgatctgagctttccttaattttcctatacaggaaagactagatcatgtggcagtttcacctattga

SEQ ID NO: 442 - DNA, 409 bp, Populus tremuloides
  misc_feature (317)..(317): n is a, c, g, or t
tgaagctgccagcatgatctggtaatgaacgtgttactctcatttatatatatattaacattaatcttatagcagtatctgtaggtagaaaaaatttaatgttgtcaaagatatatactgaatcgtggttgctaggtttgtattactagtttaggatgcatgtttttgatcttatgatgatcaattgcttgtgagttcctaggcaatgaaaacagaatatatactggtgatttttcccagtaaaattgtcgagaaaagggaattgcactaatagggaagacgcataggtaaacttgtatctaaatggtatatgtatnttccaggcaaaagggtagaaacctaatnagaaactagcttgaactcagagctataaaagtatatggttccatcaggtcatctagcagcttca

SEQ ID NO: 443 - DNA, 130 bp, Ricinus communis
aaaggtgaagctgccagcatgatctagctttggttagtgagacagctgaaagaaagatacagataacacatggtatctaagcaatagtgctaaccctagctaggtcatgctctgacagcctcactccttc

SEQ ID NO: 444 - DNA, 80 bp, Ricinus communis
tcagttgaagctgccagcatgatctaaatcttcctccctcgttgaggagagatcagatcatgtggcagtttcacccgttg

SEQ ID NO: 445 - DNA, 76 bp, Ricinus communis
atagttgaagctgccagcatgatctggagcttttctatccaggagagactagatcatgtggcagtttcacctgttg

SEQ ID NO: 446 - DNA, 96 bp, Sorghum bicolor
tgaagctgccagcatgatctagctctgagtgatcacccgagaagaacaatagttcgaggtggtctcgccttgctaggtcatgctgcggcagcctca

SEQ ID NO: 447 - DNA, 198 bp, Sorghum bicolor
tgaagctgccagcatgatctaacaacggcattgctcctccgtgtagcgccctgtgcttgcttttgcttgtctccatggagaagacagcggcaaagcttagctttgcttcgcttagcttgctggcttttcgtatgggctggcggcgggttgctgcgtgaagcttgcaagtgatggttagatcatgctgtgacagtttca

SEQ ID NO: 448 - DNA, 131 bp, Sorghum bicolor
ctttgctggtgtgagaggttgaagctgccagcatgatctggtggccggccggccggcgtctctcaagtgcgctcggatcggagacgcgtcgccagatcatgttgcagcttcactctctcgcaaccaccaaa

SEQ ID NO: 449 - DNA, 148 bp, Sorghum bicolor
gtggtgcatcctctagtagctgaagctgccagcatgatctgatgaggtgaggtttatttgctagttggtcacaggctaacagcatgatggcccaacaaatcaacgatcagatcatgctgtgcagtttcatctgctcgtggatgcacat

SEQ ID NO: 450 - DNA, 179 bp, Sorghum bicolor
agtggtgcaccacaagttggtgaagctgccagcatgatctgatgtctttatatatattaattacctctgatttctccctgactgttatggatcgatgaattcagatatgaggggaaggaagaaagaggaataatgagcatcaggtcatgctgtagtttcatccgctggtgggagcacat

SEQ ID NO: 451 - DNA, 179 bp, Sorghum bicolor
tccggtgcactagaggtggatgaagctgccagcatgatctgagaaactagtgcttgatccttttactgatttccatctagcctgcatctatatatataccttgatgcatgaatcatggtctgatgatagttaagcgagatcagatcgtctggcagtttcatcttcttatggcagcacaa

SEQ ID NO: 452 - DNA, 123 bp, Sorghum bicolor
atttgtgcaccttaagcagctgaagctgccagcatgatctgatcttaatttcttttactggcaaacttcggatgcctaagatcagatcgtgctgcgcagtttcacctgctaattggagcacag

SEQ ID NO: 453 - DNA, 90 bp, Sorghum bicolor
tgaagctgccagcatgatctgaaagcatacgagtccttcgttatcatctgatgaaagaaatagatgatcagatcatctggcagtttcatt

SEQ ID NO: 454 - DNA, 132 bp, Sorghum bicolor
agtgaagctgccagcatgatctagctttggttggcaccattggcaggcgcccacacagtggcctcttccgtgtgtgtagtgccgctctgtacctgcaaatcattgttagatcatgcatgacagcctcatttc

SEQ ID NO: 455 - DNA, 116 bp, Solanum lycopersicum
tcgtgcagcactagcagttgaagctgccagcatgatctaaactttccttttagttcaaatataattcgaggaaagatcagatcatgtggcagccttacctgtcaatgccatcacga

SEQ ID NO: 456 - DNA, 188 bp, Saccharum officinarum
agtggtgcaccacaagttggtgaagctgccagcatgatctgatggtggtatatatgaatatatgatgtctttacctctgatctctccctgactgtcaccgatccatgaatccaggatgaggggagggaagaaagagggataatgagcatcaggtcatgctgtagtttcatctgctggtgggagcacat

SEQ ID NO: 457 - DNA, 188 bp, Saccharum officinarum
agtggtgcaccacaagttggtgaagctgccagcatgatctgatggtggtatatatgaatatatgatgtctttacctctgatctctccctgactgtcacggatccatgaatccaggatgaggggagggaagaaagagggataatgagcatcaggtcatgctgtagtttcatctgctggtgggagcacat

SEQ ID NO: 458 - DNA, 139 bp, Saccharum spp
tgaagctgccagcatgatctgatggtggtatatatgaatatatgatgtctttacctctgatctctccctgactgtcacggatcgatgaatccaggatgaggggagggaataatgagcatcaggtcatgctgtagtttca

SEQ ID NO: 459 - DNA, 143 bp, Saccharum spp
ggtgaagctgccagcatgatctgatggtggtatatatgaatatatgatgtctttacctctgatctctccctgactgtcacggatcgatgaatccaggatgaggggagggaataatgagcatcaggtcatgctgtagtttcatc

SEQ ID NO: 460 - DNA, 108 bp, Triticum aestivum
ctgcccaagggaacgagtgaagctgccagcatgatctagctccgagtgatcaaacaagaaacgctgcggcagcctcacttcttcccgccgttgggcacaactacttct

SEQ ID NO: 461 - DNA, 90 bp, Triticum aestivum
ctgcccaagggaacgagtgaagctgacagcatgatctatctccgagtgatcaaacaagaaacgctgcggcagcctcacttcttcccggcg

SEQ ID NO: 462 - DNA, 111 bp, Theobroma cacao
gccgtgcacccactagcagttgaagctgccagcatgatctaaacttccttctctgtcgagaggatagattggatcatgtggtagcttcacctgttgttgggatcacgaaga

SEQ ID NO: 463 - DNA, 138 bp, Theobroma cacao
gaattctgcagtggaaaaagtgaagctgccagcatgatctatctttggttagtgagtgaaagggggtgctaaggctatgttgctaaccctagctaggtcatgctctgacagcctcactccttcctacttggggaccca

SEQ ID NO: 464 - DNA, 112 bp, Theobroma cacao
tatggtgctccaccatcagttgaagctgccagcatgatcttaattttccttctttttatcaaggaaagatcagatcatatggcagtttcacctgttgctgcttgcacaatcc

SEQ ID NO: 465 - DNA, 351 bp, Vitis vinifera
tttgagaggttgaagctgccagcatgatctggtgaaacaaacaccatctctttcttctctaaccccatgtctggattcgtccaccgatccattattatagaccaggccgcccgtttcccatgtagtgatcgataattaggctcggggttttcactttttagtgggatctaatccttaggatggatgtttgtatgggtggtatatatcatggtgaggtctgttttctattttaattctaacggggttttgatttagctgagggggtataattcatagcctaattccaaaacctaactccatagagatagggttccatgatcaggtcatcttgcagcttcaatcactcactca

SEQ ID NO: 466 - DNA, 99 bp, Vitis vinifera
caatagcagttgaagctgccagcatgatctaagcttttctgttgcccaccctttctccaggaaagactagatcatgtggcagtttcacctgttgatgga

SEQ ID NO: 467 - DNA, 91 bp, Vitis vinifera
cagtagcagttgaagctgccagcatgatctcaacttccctatacaagtcaaggaaagatcagatcatgtggtagcctcacctgttgatggg

SEQ ID NO: 468 - DNA, 115 bp, Vitis vinifera
agggaataagtgaagctgccagcatgatctagctttggctagggatacagagaaagagagagatcagagctaaccctagctaggtcatgccctgacagcctcactccttccttct

SEQ ID NO: 469 - DNA, 90 bp, Vitis vinifera
cactatcagttgaagctgccagcatgatctaaacttgcttccctttgtgaacagagatcagatcatgtggcagtttcacctgttgttggt

SEQ ID NO: 470 - DNA, 190 bp, Zea mays
tgctcttgcgaatgagtgaagctgccagcatgatctagctctgatttggttggcaccatattagcaggcgtccacgcacagctagactagagtggcctcgcgcgctctcgtctggtctgtgtctcgctttgtgcctgcaaatcgttgttagatcatgcatgacagcctcattccttcacaattctggggc

SEQ ID NO: 471 - DNA, 190 bp, Zea mays; identical to SEQ ID NO: 470

SEQ ID NO: 472 - DNA, 127 bp, Zea mays
agtgcccaagataaagggtgaagctgccagcatgatctaacgacggcattgctctgctgctgcagtgaggcttgcgagtgatggttagatcatgctgtgacagtttcactctttccctttgggcaca

SEQ ID NO: 473 - DNA, 132 bp, Zea mays
tgcccaagggaacgagtgaagctgccagcatgatctagctcggagtgatcacgcgaggagaacaatagctcgaggtggtcatgccttgctagatcatgctgtggcagcctcacttcttcccgtccttgggca

SEQ ID NO: 474 - DNA, 133 bp, Zea mays
tgcccaagggaacgagtgaagctgccagcatgatctagctctgagtgatcacccgaaaaagaacaatagttctaggtggtcatgccttgctaggtcatgctgctgcagcctcacttcttcccgtcgttgggca

SEQ ID NO: 475 - DNA, 133 bp, Zea mays; identical to SEQ ID NO: 474

SEQ ID NO: 476 - DNA, 119 bp, Zea mays
ttggtgtgtcctctagtagctgaagctgccagcatgatctgaggtgtccacagcatatatatggaagcagctagcgatcagatcatgctgtgcagtttcatctgctcgtggacgcacac

SEQ ID NO: 477 - DNA, 119 bp, Zea mays; identical to SEQ ID NO: 476

SEQ ID NO: 478 - DNA, 119 bp, Zea mays
cgtgcaccttattaagcagctgaagctgccagcatgatctgatctttcgtttactggcaactttggatacctaagatccagatcgtgctgcgcagtttcacctgctaattggagcacag

SEQ ID NO: 479 - DNA, 243 bp, Zea mays
agtggtgcaccacgagttggtgaagctgccagcatgatctggttatgatggtggtggtatatgtaagatggatgtaatctatactactaccggcccctgtcactctctctctctcccccgtccctgactgtcatatatggatcgacgaatccaagatgagaggggaagggagagagagagagggtaattaatgagcaccaggaccaggtcatgctgtagtttcatctgctggtggccgcacat

SEQ ID NO: 480 - DNA, 143 bp, Zea mays
actttgctgctgtgagaggttgaagctgccagcatgatctggctgctcagacgccggcgggcgtctcgagtgctcgctcgatcgtcggtgacgcttggattcaccagatcatgttgcagcttcactctctcgcagccagcaaa

SEQ ID NO: 481 - DNA, 130 bp, Zea mays
acttcgctggtgtgagagcttgaagctgccagcatgatctggctrctcaaacgccgccggcctcccaagtgctcgatcggtggcgcttcaccagatcatgttgcagcttcactctctcgcaaccagcgaa

SEQ ID NO: 482 - DNA, 109 bp, Zea mays
atgaagctgccagcatgatctgaaaccatacgtgttctttgattcccatctgaagaaagagttggctttcatggagaaccgacggtcagatcatgtggcagtttcattt

SEQ ID NO: 483 - DNA, 91 bp, Zea mays
tgaagctgccagcatgatctggctgctcaaacgccgccggcctcccaagtgctcgatcggtggcgcttcaccagatcatgttgcagcttca

SEQ ID NO: 484 - DNA, 80 bp, Zea mays
tgaagctgccagcatgatctgatctttcgtttactggcaactttggatacctaagatccagatcgtgctgcgcagtttca

SEQ ID NO: 485 - DNA, 80 bp, Zea mays
tgaagctgccagcatgatctgaggtgtccacagcatatatatggaagcagctagcgatcagatcatgctgtgcagtttca

SEQ ID NO: 486 - DNA, 221 bp, Zea mays
gagtttgcagatctcagtttggtagcttcttctattccactggccatcacttgctttgatttcttccgtttcttataggttgtacaactttctgttctttggatctgagattgaataatcactcatctacacctagtcatggtattttatgcaacatgttctagctagcctggaactgcctgctcaagggaacgagtgaagctgccagcatgatctagctc

SEQ ID NO: 487 - DNA, 160 bp, Zea mays
gagtgaagctgccagcatgatctagctctgatttggttggcaccatattagcaggcgtccacgcacagctagactagagtggcctcgcgcgctctcgtctggtctgtgtctcgctttgtgcctgcaaatcgttgttagatcatgcatgacagcctcattc

SEQ ID NO: 488 - DNA, 103 bp, Zea mays
gagtgaagctgccagcatgatctagctctgagtgatcacccgaaaaagaacaatagttctaggtggtcatgccttgctaggtcatgctgctgcagcctcactt

SEQ ID NO: 489 - DNA, 102 bp, Zea mays
gagtgaagctgccagcatgatctagctcggagtgatcacgcgaggagaacaatagctcgaggtggtcatgccttgctagatcatgctgtggcagcctcactt

SEQ ID NO: 490 - DNA, 102 bp, Zea mays
aaagggtgaagctgccagcatgatctaacgacggcattgctctgctgctgcagtgaggcttgcgagtgatggttagatcatgctgtgacagtttcactcttt

SEQ ID NO: 491 - DNA, 99 bp, Zea mays
gagtgaagctgccagcatgatctagctcggagtgatcacgcgaggagacatagctcgaggtggtcatgccttgctagatcatgctgtggcagctcactt

SEQ ID NO: 492 - DNA, 92 bp, Zea mays
gtgaagctgccagcatgatctaacgacggcattgctctgctgctgcagtgaggcttgcgagtgatggttagatcatgctgtgacagtttcac

SEQ ID NO: 493 - DNA, 262 bp, Zea mays
tgaagctgccacatgatctgatgacgcagagtcatgcatatgcattgcatccagcaagctccatgcgtgcgtgcatggccgaatggccgaagagactagctagtccatctctccaaggccatccacgtgtgagaattcaattcctcgtggatcagatcaggctgttgttgacaactgcatgccgcacctgcactacagcaacccaaggcataggtagctagctaggtttcggtggtcagatcagatcaggctggcagcttca

SEQ ID NO: 494 - DNA, 118 bp, Arachis hypogaea; identical to SEQ ID NO: 370

SEQ ID NO: 495 - DNA, 138 bp, Arabidopsis lyrata; identical to SEQ ID NO: 371

SEQ ID NO: 496 - DNA, 195 bp, Arabidopsis lyrata; identical to SEQ ID NO: 372

SEQ ID NO: 497 - DNA, 159 bp, Arabidopsis lyrata; identical to SEQ ID NO: 373

SEQ ID NO: 498 - DNA, 327 bp, Arabidopsis lyrata; identical to SEQ ID NO: 374

SEQ ID NO: 499 - DNA, 68 bp, Aquilegia coerulea; identical to SEQ ID NO: 375

SEQ ID NO: 500 - DNA, 138 bp, Arabidopsis thaliana; identical to SEQ ID NO: 2

SEQ ID NO: 501 - DNA, 109 bp, Arabidopsis thaliana; identical to SEQ ID NO: 376

SEQ ID NO: 502 - DNA, 377 bp, Arabidopsis thaliana; identical to SEQ ID NO: 378

SEQ ID NO: 503 - DNA, 342 bp, Arabidopsis thaliana; identical to SEQ ID NO: 379

SEQ ID NO: 504 - DNA, 91 bp, Brachypodium distachyon; identical to SEQ ID NO: 380

SEQ ID NO: 505 - DNA, 91 bp, Brachypodium distachyon; identical to SEQ ID NO: 380

SEQ ID NO: 506 - DNA, 190 bp, Brachypodium distachyon; identical to SEQ ID NO: 382

SEQ ID NO: 507 - DNA, 159 bp, Brachypodium distachyon; identical to SEQ ID NO: 383

SEQ ID NO: 508 - DNA, 204 bp, Brachypodium distachyon; identical to SEQ ID NO: 384

SEQ ID NO: 509 - DNA, 132 bp, Brassica napus; identical to SEQ ID NO: 385

SEQ ID NO: 510 - DNA, 124 bp, Brassica napus; identical to SEQ ID NO: 386

SEQ ID NO: 511 - DNA, 134 bp, Brassica napus; identical to SEQ ID NO: 387

SEQ ID NO: 512 - DNA, 134 bp, Brassica rapa; identical to SEQ ID NO: 387

SEQ ID NO: 513 - DNA, 132 bp, Brassica rapa; identical to SEQ ID NO: 385

SEQ ID NO: 514 - DNA, 132 bp, Brassica rapa; identical to SEQ ID NO: 385

SEQ ID NO: 515 - DNA, 124 bp, Brassica rapa; identical to SEQ ID NO: 386

SEQ ID NO: 516 - DNA, 121 bp, Citrus clementine; identical to SEQ ID NO: 392

SEQ ID NO: 517 - DNA, 202 bp, Citrus clementine; identical to SEQ ID NO: 393

SEQ ID NO: 518 - DNA, 96 bp, Citrus clementina; identical to SEQ ID NO: 394

SEQ ID NO: 519 - DNA, 103 bp, Citrus sinensis; identical to SEQ ID NO: 395

SEQ ID NO: 520 - DNA, 123 bp, Citrus sinensis; identical to SEQ ID NO: 396

SEQ ID NO: 521 - DNA, 341 bp, Citrus sinensis; identical to SEQ ID NO: 397
  misc_feature (269)..(269): n is a, c, g, or t

SEQ ID NO: 522 - DNA, 121 bp, Citrus trifoliata; identical to SEQ ID NO: 392

SEQ ID NO: 523 - DNA, 105 bp, Gossypium hirsutum; identical to SEQ ID NO: 399

SEQ ID NO: 524 - DNA, 119 bp, Glycine max; identical to SEQ ID NO: 400

SEQ ID NO: 525 - DNA, 121 bp, Glycine max; identical to SEQ ID NO: 401

SEQ ID NO: 526 - DNA, 375 bp, Glycine max; identical to SEQ ID NO: 402

SEQ ID NO: 527 - DNA, 121 bp, Glycine max; identical to SEQ ID NO: 403

SEQ ID NO: 528 - DNA, 109 bp, Glycine max; identical to SEQ ID NO: 404

SEQ ID NO: 529 - DNA, 109 bp, Glycine max; identical to SEQ ID NO: 405

SEQ ID NO: 530 - DNA, 78 bp, Glycine max; identical to SEQ ID NO: 406

SEQ ID NO: 531 - DNA, 151 bp, Glycine max; identical to SEQ ID NO: 407

SEQ ID NO: 532 - DNA, 151 bp, Glycine max; identical to SEQ ID NO: 408

SEQ ID NO: 533 - DNA, 78 bp, Glycine max; identical to SEQ ID NO: 406

SEQ ID NO: 534 - DNA, 64 bp, Glycine max; identical to SEQ ID NO: 410

SEQ ID NO: 535 - DNA, 109 bp, Glycine max; identical to SEQ ID NO: 411

SEQ ID NO: 536 - DNA, 80 bp, Glycine soja; identical to SEQ ID NO: 412

SEQ ID NO: 537 - DNA, 271 bp, Ipomoea nil; identical to SEQ ID NO: 413

SEQ ID NO: 538 - DNA, 108 bp, Lotus japonicus; identical to SEQ ID NO: 414

SEQ ID NO: 539 - DNA, 320 bp, Medicago truncatula; identical to SEQ ID NO: 415

SEQ ID NO: 540 - DNA, 207 bp, Medicago truncatula; identical to SEQ ID NO: 416

SEQ ID NO: 541 - DNA, 141 bp, Oryza sativa; identical to SEQ ID NO: 417

SEQ ID NO: 542 - DNA, 141 bp, Oryza sativa; identical to SEQ ID NO: 417

SEQ ID NO: 543 - DNA, 163 bp, Oryza sativa; identical to SEQ ID NO: 419

SEQ ID NO: 544 - DNA, 163 bp, Oryza sativa; identical to SEQ ID NO: 420

SEQ ID NO: 545 - DNA, 110 bp, Oryza sativa; identical to SEQ ID NO: 421

SEQ ID NO: 546 - DNA, 273 bp, Oryza sativa; identical to SEQ ID NO: 422

SEQ ID NO: 547 - DNA, 113 bp, Oryza sativa; identical to SEQ ID NO: 423

SEQ ID NO: 548 - DNA, 82 bp, Oryza sativa; identical to SEQ ID NO: 424

SEQ ID NO: 549 - DNA, 120 bp, Oryza sativa; identical to SEQ ID NO: 425

SEQ ID NO: 550 - DNA, 201 bp, Oryza sativa; identical to SEQ ID NO: 426

SEQ ID NO: 551 - DNA, 160 bp, Oryza sativa; identical to SEQ ID NO: 427

SEQ ID NO: 552 - DNA, 100 bp, Oryza sativa; identical to SEQ ID NO: 428

SEQ ID NO: 553 - DNA, 90 bp, Oryza sativa; identical to SEQ ID NO: 429

SEQ ID NO: 554 - DNA, 69 bp, Phaseolus coccineus; identical to SEQ ID NO: 430

SEQ ID NO: 555 - DNA, 108 bp, Populus tremula x Populus tremuloides; identical to SEQ ID NO: 431

SEQ ID NO: 556 - DNA, 431 bp, Populus tremula x Populus tremuloides; identical to SEQ ID NO: 432
  misc_feature (327)..(327): n is a, c, g, or t

SEQ ID NO: 557 - DNA, 82 bp, Physcomitrella patens; identical to SEQ ID NO: 433

SEQ ID NO: 558 - DNA, 89 bp, Populus trichocarpa; identical to SEQ ID NO: 434

SEQ ID NO: 559 - DNA, 96 bp, Populus trichocarpa; identical to SEQ ID NO: 435

SEQ ID NO: 560 - DNA, 89 bp, Populus trichocarpa; identical to SEQ ID NO: 436

SEQ ID NO: 561 - DNA, 105 bp, Populus trichocarpa; identical to SEQ ID NO: 437

SEQ ID NO: 562 - DNA, 91 bp, Populus trichocarpa; identical to SEQ ID NO: 438

SEQ ID NO: 563 - DNA, 87 bp, Populus trichocarpa; identical to SEQ ID NO: 439

SEQ ID NO: 564 - DNA, 86 bp, Populus trichocarpa; identical to SEQ ID NO: 440

SEQ ID NO: 565 - DNA, 91 bp, Populus trichocarpa; identical to SEQ ID NO: 441

SEQ ID NO: 566 - DNA, 409 bp, Populus tremuloides; identical to SEQ ID NO: 442
  misc_feature (317)..(317): n is a, c, g, or t

SEQ ID NO: 567 - DNA, 130 bp, Ricinus communis; identical to SEQ ID NO: 443

SEQ ID NO: 568 - DNA, 80 bp, Ricinus communis; identical to SEQ ID NO: 444

SEQ ID NO: 569 - DNA, 76 bp, Ricinus communis; identical to SEQ ID NO: 445

SEQ ID NO: 570 - DNA, 96 bp, Sorghum bicolor; identical to SEQ ID NO: 446

SEQ ID NO: 571 - DNA, 198 bp, Sorghum bicolor; identical to SEQ ID NO: 447

SEQ ID NO: 572 - DNA, 131 bp, Sorghum bicolor; identical to SEQ ID NO: 448

SEQ ID NO: 573 - DNA, 148 bp, Sorghum bicolor; identical to SEQ ID NO: 449

SEQ ID NO: 574 - DNA, 179 bp, Sorghum bicolor; identical to SEQ ID NO: 450

SEQ ID NO: 575 - DNA, 179 bp, Sorghum bicolor; identical to SEQ ID NO: 451

SEQ ID NO: 576 - DNA, 123 bp, Sorghum bicolor; identical to SEQ ID NO: 452

SEQ ID NO: 577 - DNA, 90 bp, Sorghum bicolor; identical to SEQ ID NO: 453

SEQ ID NO: 578 - DNA, 132 bp, Sorghum bicolor; identical to SEQ ID NO: 454

SEQ ID NO: 579 - DNA, 116 bp, Solanum lycopersicum; identical to SEQ ID NO: 455

SEQ ID NO: 580 - DNA, 188 bp, Saccharum officinarum; identical to SEQ ID NO: 456

SEQ ID NO: 581 - DNA, 188 bp, Saccharum officinarum; identical to SEQ ID NO: 457

SEQ ID NO: 582 - DNA, 139 bp, Saccharum spp; identical to SEQ ID NO: 458

SEQ ID NO: 583 - DNA, 143 bp, Saccharum spp; identical to SEQ ID NO: 459

SEQ ID NO: 584 - DNA, 108 bp, Triticum aestivum; identical to SEQ ID NO: 460

SEQ ID NO: 585 - DNA, 90 bp, Triticum aestivum; identical to SEQ ID NO: 461

SEQ ID NO: 586 - DNA, 111 bp, Theobroma cacao; identical to SEQ ID NO: 462

SEQ ID NO: 587 - DNA, 138 bp, Theobroma cacao; identical to SEQ ID NO: 463

SEQ ID NO: 588 - DNA, 112 bp, Theobroma cacao; identical to SEQ ID NO: 464

SEQ ID NO: 589 - DNA, 351 bp, Vitis vinifera; identical to SEQ ID NO: 465

SEQ ID NO: 590 - DNA, 99 bp, Vitis vinifera; identical to SEQ ID NO: 466

SEQ ID NO: 591 - DNA, 91 bp, Vitis vinifera; identical to SEQ ID NO: 467

SEQ ID NO: 592 - DNA, 115 bp, Vitis vinifera; identical to SEQ ID NO: 468

SEQ ID NO: 593 - DNA, 90 bp, Vitis vinifera; identical to SEQ ID NO: 469

SEQ ID NO: 594 - DNA, 190 bp, Zea mays; identical to SEQ ID NO: 470

SEQ ID NO: 595 - DNA, 127 bp, Zea mays; identical to SEQ ID NO: 472

SEQ ID NO: 596 - DNA, 132 bp, Zea mays; identical to SEQ ID NO: 473

SEQ ID NO: 597 - DNA, 133 bp, Zea mays; identical to SEQ ID NO: 474

SEQ ID NO: 598 - DNA, 119 bp, Zea mays; identical to SEQ ID NO: 476

SEQ ID NO: 599 - DNA, 119 bp, Zea mays; identical to SEQ ID NO: 478

SEQ ID NO: 600 - DNA, 243 bp, Zea mays; identical to SEQ ID NO: 479

SEQ ID NO: 601 - DNA, 143 bp, Zea mays; identical to SEQ ID NO: 480

SEQ ID NO: 602 - DNA, 130 bp, Zea mays; identical to SEQ ID NO: 481

SEQ ID NO: 603 - DNA, 109 bp, Zea mays; identical to SEQ ID NO: 482

SEQ ID NO: 604 - DNA, 91 bp, Zea mays; identical to SEQ ID NO: 483

SEQ ID NO: 605 - DNA, 80 bp, Zea mays; identical to SEQ ID NO: 484

SEQ ID NO: 606 - DNA, 80 bp, Zea mays; identical to SEQ ID NO: 485

SEQ ID NO: 607 - DNA, 221 bp, Zea mays; identical to SEQ ID NO: 486

SEQ ID NO: 608 - DNA, 160 bp, Zea mays; identical to SEQ ID NO: 487

SEQ ID NO: 609 - DNA, 103 bp, Zea mays; identical to SEQ ID NO: 488

SEQ ID NO: 610 - DNA, 102 bp, Zea mays; identical to SEQ ID NO: 489

SEQ ID NO: 611 - DNA, 102 bp, Zea mays; identical to SEQ ID NO: 490

SEQ ID NO: 612 - DNA, 99 bp, Zea mays; identical to SEQ ID NO: 491

SEQ ID NO: 613 - DNA, 92 bp, Zea mays; identical to SEQ ID NO: 492

SEQ ID NO: 614 - DNA, 262 bp, Zea mays; identical to SEQ ID NO: 493

SEQ ID NO: 615 - DNA, 118 bp, Arachis hypogaea; identical to SEQ ID NO: 370

SEQ ID NO: 616 - DNA, 138 bp, Arabidopsis lyrata; identical to SEQ ID NO: 371

SEQ ID NO: 617 - DNA, 195 bp, Arabidopsis lyrata; identical to SEQ ID NO: 372

SEQ ID NO: 618 - DNA, 159 bp, Arabidopsis lyrata; identical to SEQ ID NO: 373

SEQ ID NO: 619 - DNA, 327 bp, Arabidopsis lyrata; identical to SEQ ID NO: 374

SEQ ID NO: 620 - DNA, 68 bp, Aquilegia coerulea; identical to SEQ ID NO: 375

SEQ ID NO: 621 - DNA, 138 bp, Arabidopsis thaliana; identical to SEQ ID NO: 2

SEQ ID NO: 622 - DNA, 109 bp, Arabidopsis thaliana; identical to SEQ ID NO: 376

SEQ ID NO: 623 - DNA, 160 bp, Arabidopsis thaliana; identical to SEQ ID NO: 377

SEQ ID NO: 624 - DNA, 342 bp, Arabidopsis thaliana; identical to SEQ ID NO: 379

SEQ ID NO: 625 - DNA, 91 bp, Brachypodium distachyon; identical to SEQ ID NO: 380

SEQ ID NO: 626 - DNA, 91 bp, Brachypodium distachyon; identical to SEQ ID NO: 380

SEQ ID NO: 627 - DNA, 190 bp, Brachypodium distachyon; identical to SEQ ID NO: 382

SEQ ID NO: 628 - DNA, 159 bp, Brachypodium distachyon; identical to SEQ ID NO: 383

SEQ ID NO: 629 - DNA, 204 bp, Brachypodium distachyon; identical to SEQ ID NO: 384

SEQ ID NO: 630 - DNA, 132 bp, Brassica napus; identical to SEQ ID NO: 385

SEQ ID NO: 631 - DNA, 124 bp, Brassica napus; identical to SEQ ID NO: 386

SEQ ID NO: 632 - DNA, 134 bp, Brassica napus; identical to SEQ ID NO: 387

SEQ ID NO: 633 - DNA, 134 bp, Brassica rapa; identical to SEQ ID NO: 387

SEQ ID NO: 634 - DNA, 132 bp, Brassica rapa; identical to SEQ ID NO: 385

SEQ ID NO: 635 - DNA, 132 bp, Brassica rapa; identical to SEQ ID NO: 385

SEQ ID NO: 636 - DNA, 124 bp, Brassica rapa; identical to SEQ ID NO: 386

SEQ ID NO: 637 - DNA, 121 bp, Citrus clementine; identical to SEQ ID NO: 392

SEQ ID NO: 638 - DNA, 202 bp, Citrus clementine; identical to SEQ ID NO: 393

SEQ ID NO: 639 - DNA, 96 bp, Citrus clementina; identical to SEQ ID NO: 394

SEQ ID NO: 640 - DNA, 103 bp, Citrus sinensis; identical to SEQ ID NO: 395

SEQ ID NO: 641 - DNA, 123 bp, Citrus sinensis; identical to SEQ ID NO: 396

SEQ ID NO: 642 - DNA, 341 bp, Citrus sinensis; identical to SEQ ID NO: 397
  misc_feature (269)..(269): n is a, c, g, or t

SEQ ID NO: 643 - DNA, 121 bp, Citrus trifoliata; identical to SEQ ID NO: 392

SEQ ID NO: 644 - DNA, 105 bp, Gossypium hirsutum; identical to SEQ ID NO: 399

SEQ ID NO: 645 - DNA, 119 bp, Glycine max; identical to SEQ ID NO: 400

SEQ ID NO: 646 - DNA, 121 bp, Glycine max; identical to SEQ ID NO: 401

SEQ ID NO: 647 - DNA, 375 bp, Glycine max; identical to SEQ ID NO: 402

SEQ ID NO: 648 - DNA, 121 bp, Glycine max; identical to SEQ ID NO: 403

SEQ ID NO: 649 - DNA, 109 bp, Glycine max; identical to SEQ ID NO: 404

SEQ ID NO: 650 - DNA, 109 bp, Glycine max; identical to SEQ ID NO: 405

SEQ ID NO: 651 - DNA, 78 bp, Glycine max; identical to SEQ ID NO: 406

SEQ ID NO: 652 - DNA, 151 bp, Glycine max; identical to SEQ ID NO: 407

SEQ ID NO: 653 - DNA, 151 bp, Glycine max; identical to SEQ ID NO: 408

SEQ ID NO: 654 - DNA, 78 bp, Glycine max; identical to SEQ ID NO: 406

SEQ ID NO: 655 - DNA, 64 bp, Glycine max; identical to SEQ ID NO: 410

SEQ ID NO: 656 - DNA, 109 bp, Glycine max; identical to SEQ ID NO: 411

SEQ ID NO: 657 - DNA, 80 bp, Glycine soja; identical to SEQ ID NO: 412

SEQ ID NO: 658 - DNA, 271 bp, Ipomoea nil; identical to SEQ ID NO: 413

SEQ ID NO: 659 - DNA, 108 bp, Lotus japonicus; identical to SEQ ID NO: 414

SEQ ID NO: 660 - DNA, 320 bp, Medicago truncatula; identical to SEQ ID NO: 415

SEQ ID NO: 661 - DNA, 207 bp, Medicago truncatula; identical to SEQ ID NO: 416

SEQ ID NO: 662 - DNA, 141 bp, Oryza sativa; identical to SEQ ID NO: 417

SEQ ID NO: 663 - DNA, 141 bp, Oryza sativa; identical to SEQ ID NO: 417

SEQ ID NO: 664 - DNA, 163 bp, Oryza sativa; identical to SEQ ID NO: 419

SEQ ID NO: 665 - DNA, 163 bp, Oryza sativa; identical to SEQ ID NO: 420

SEQ ID NO: 666 - DNA, 110 bp, Oryza sativa; identical to SEQ ID NO: 421

SEQ ID NO: 667 - DNA, 273 bp, Oryza sativa; identical to SEQ ID NO: 422

SEQ ID NO: 668 - DNA, 113 bp, Oryza sativa; identical to SEQ ID NO: 423

SEQ ID NO: 669 - DNA, 82 bp, Oryza sativa; identical to SEQ ID NO: 424

SEQ ID NO: 670 - DNA, 120 bp, Oryza sativa; identical to SEQ ID NO: 425

SEQ ID NO: 671 - DNA, 201 bp, Oryza sativa; identical to SEQ ID NO: 426

SEQ ID NO: 672 - DNA, 160 bp, Oryza sativa; identical to SEQ ID NO: 427

SEQ ID NO: 673 - DNA, 100 bp, Oryza sativa; identical to SEQ ID NO: 428

SEQ ID NO: 674 - DNA, 90 bp, Oryza sativa; identical to SEQ ID NO: 429

SEQ ID NO: 675 - DNA, 69 bp, Phaseolus coccineus; identical to SEQ ID NO: 430

SEQ ID NO: 676 - DNA, 108 bp, Populus tremula x Populus tremuloides; identical to SEQ ID NO: 431

SEQ ID NO: 677 - DNA, 431 bp, Populus tremula x Populus tremuloides; identical to SEQ ID NO: 432
  misc_feature (327)..(327): n is a, c, g, or t

SEQ ID NO: 678 - DNA, 82 bp, Physcomitrella patens; identical to SEQ ID NO: 433

SEQ ID NO: 679 - DNA, 89 bp, Populus trichocarpa; identical to SEQ ID NO: 434

SEQ ID NO: 680 - DNA, 96 bp, Populus trichocarpa; identical to SEQ ID NO: 435

SEQ ID NO: 681 - DNA, 89 bp, Populus trichocarpa; identical to SEQ ID NO: 436

SEQ ID NO: 682 - DNA, 105 bp, Populus trichocarpa; identical to SEQ ID NO: 437

SEQ ID NO: 683 - DNA, 91 bp, Populus trichocarpa; identical to SEQ ID NO: 438

SEQ ID NO: 684 - DNA, 87 bp, Populus trichocarpa; identical to SEQ ID NO: 439

SEQ ID NO: 685 - DNA, 86 bp, Populus trichocarpa; identical to SEQ ID NO: 440

SEQ ID NO: 686 - DNA, 91 bp, Populus trichocarpa; identical to SEQ ID NO: 441

SEQ ID NO: 687 - DNA, 409 bp, Populus tremuloides; identical to SEQ ID NO: 442
  misc_feature (317)..(317): n is a, c, g, or t

SEQ ID NO: 688 - DNA, 130 bp, Ricinus communis; identical to SEQ ID NO: 443

SEQ ID NO: 689 - DNA, 80 bp, Ricinus communis; identical to SEQ ID NO: 444

SEQ ID NO: 690 - DNA, 76 bp, Ricinus communis; identical to SEQ ID NO: 445

SEQ ID NO: 691 - DNA, 96 bp, Sorghum bicolor; identical to SEQ ID NO: 446

SEQ ID NO: 692 - DNA, 198 bp, Sorghum bicolor; identical to SEQ ID NO: 447

SEQ ID NO: 693 - DNA, 131 bp, Sorghum bicolor; identical to SEQ ID NO: 448

SEQ ID NO: 694 - DNA, 148 bp, Sorghum bicolor; identical to SEQ ID NO: 449

SEQ ID NO: 695 - DNA, 179 bp, Sorghum bicolor; identical to SEQ ID NO: 450

SEQ ID NO: 696 - DNA, 179 bp, Sorghum bicolor; identical to SEQ ID NO: 451

SEQ ID NO: 697 - DNA, 123 bp, Sorghum bicolor; identical to SEQ ID NO: 452

SEQ ID NO: 698 - DNA, 90 bp, Sorghum bicolor; identical to SEQ ID NO: 453

SEQ ID NO: 699 - DNA, 132 bp, Sorghum bicolor; identical to SEQ ID NO: 454

SEQ ID NO: 700 - DNA, 116 bp, Solanum lycopersicum; identical to SEQ ID NO: 455

SEQ ID NO: 701 - DNA, 188 bp, Saccharum officinarum; identical to SEQ ID NO: 456

SEQ ID NO: 702 - DNA, 188 bp, Saccharum officinarum; identical to SEQ ID NO: 457

SEQ ID NO: 703 - DNA, 139 bp, Saccharum spp; identical to SEQ ID NO: 458

SEQ ID NO: 704 - DNA, 143 bp, Saccharum spp; identical to SEQ ID NO: 459

SEQ ID NO: 705 - DNA, 108 bp, Triticum aestivum; identical to SEQ ID NO: 460

SEQ ID NO: 706 - DNA, 90 bp, Triticum aestivum; identical to SEQ ID NO: 461

SEQ ID NO: 707 - DNA, 111 bp, Theobroma cacao; identical to SEQ ID NO: 462

SEQ ID NO: 708 - DNA, 138 bp, Theobroma cacao; identical to SEQ ID NO: 463

SEQ ID NO: 709 - DNA, 112 bp, Theobroma cacao; identical to SEQ ID NO: 464

SEQ ID NO: 710 - DNA, 351 bp, Vitis vinifera; identical to SEQ ID NO: 465

SEQ ID NO: 711 - DNA, 99 bp, Vitis vinifera; identical to SEQ ID NO: 466

SEQ ID NO: 712 - DNA, 91 bp, Vitis vinifera; identical to SEQ ID NO: 467

SEQ ID NO: 713 - DNA, 115 bp, Vitis vinifera; identical to SEQ ID NO: 468

SEQ ID NO: 714 - DNA, 90 bp, Vitis vinifera; identical to SEQ ID NO: 469

SEQ ID NO: 715 - DNA, 190 bp, Zea mays; identical to SEQ ID NO: 470

SEQ ID NO: 716 - DNA, 127 bp, Zea mays; identical to SEQ ID NO: 472

SEQ ID NO: 717 - DNA, 132 bp, Zea mays; identical to SEQ ID NO: 473

SEQ ID NO: 718 - DNA, 133 bp, Zea mays; identical to SEQ ID NO: 474

SEQ ID NO: 719 - DNA, 119 bp, Zea mays; identical to SEQ ID NO: 476

SEQ ID NO: 720 - DNA, 119 bp, Zea mays; identical to SEQ ID NO: 478

SEQ ID NO: 721 - DNA, 243 bp, Zea mays; identical to SEQ ID NO: 479

SEQ ID NO: 722 - DNA, 143 bp, Zea mays; identical to SEQ ID NO: 480

SEQ ID NO: 723 - DNA, 130 bp, Zea mays; identical to SEQ ID NO: 481

SEQ ID NO: 724 - DNA, 109 bp, Zea mays; identical to SEQ ID NO: 482

SEQ ID NO: 725 - DNA, 91 bp, Zea mays; identical to SEQ ID NO: 483

SEQ ID NO: 726 - DNA, 80 bp, Zea mays; identical to SEQ ID NO: 484

SEQ ID NO: 727 - DNA, 80 bp, Zea mays; identical to SEQ ID NO: 485

SEQ ID NO: 728 - DNA, 221 bp, Zea mays; identical to SEQ ID NO: 486

SEQ ID NO: 729 - DNA, 160 bp, Zea mays; identical to SEQ ID NO: 487

SEQ ID NO: 730 - DNA, 103 bp, Zea mays; identical to SEQ ID NO: 488

SEQ ID NO: 731 - DNA, 102 bp, Zea mays; identical to SEQ ID NO: 489

SEQ ID NO: 732 - DNA, 102 bp, Zea mays; identical to SEQ ID NO: 490

SEQ ID NO: 733 - DNA, 99 bp, Zea mays; identical to SEQ ID NO: 491

SEQ ID NO: 734 - DNA, 92 bp, Zea mays; identical to SEQ ID NO: 492

SEQ ID NO: 735 - DNA, 262 bp, Zea mays; identical to SEQ ID NO: 493

SEQ ID NO: 736 - DNA, 948 bp, Artificial sequence
  Os.Gos2 constitutive promoter
aatccgaaaagtttctgcaccgttttcacgtcctaactaacaatatagggaacgtgtgctaaatataaaatgagaccttatatatgtagcgctgataactagaactatgtaagaaaaactcatccacctactttagtggcaatcgggctaaataaaaaagagtcgctacactagtttcgttttccttagtaattaagtgggaaaatgaaatcattattgcttagaatatacgttcacatctctgtcatgaagttaaattattcgaggtagccataattgtcatcaaactcttcttgaataaaaaaatctttctagctgaactcaatgggtaaagagagatatttttttttaaaaaaaaatagaatgaagatattctgaacgtatcggcaaagatttaaacatataattatataattttatagtttgtgcattcgttatatcgcacgtcattaaggacatgtcttactccatctcaatttttatttagtaattaaagacaattgacttatttttattatttatcttttttcgattagatgcaaggtacttacgcacacactttgtgctcatgtgcatgtgtgagtgcacctcctcaatacacgttcaactagcgacacatctccaatatcactcgcctatttaatacatttaggtagcaatatctgaattcaagcactccaccatcaccagaccacttttaataatatctaaaatacaaaaaataattttacagaatagcatgaaaagtatgaaacgaactatttaggtttttcacatacaaaaaaaaaaagaattttgctcgtgcgcgagcgccaatctcccatattgggcacacaggcaacaacagagtggctgcccacagaacaacccacaaaaaacgatgatctaacggaggacagcaagtccgcaacaaccttttaacagcaggctttgcggccaggagagagg

SEQ ID NO: 737 - DNA, 363 bp, Artificial sequence
  Zm.H2a meristem promoter
catacaaattatatatatatattttaaatatcaaatctttataagaatgatgatccactgtccactgctgcccacttcccacgcccaaaacaagttcacctccgtggcgcgtgttccgaaaagtcctcttgttgtgggcgggagaatggaggcgtaatatttcggcgtccccgaaatttgcttgcaccttattggccgagccacccctcccacggatcgtgccctgctggcaacattgcagccatcggtgcccctctagatccaaccatccactgtcctcgcacgcggatccacgggcccaccagcctcggcagccgagttgtttaaactttataaatacccgtcgccgcctgctactttccc

SEQ ID NO: 738 - DNA, 1100 bp, Artificial sequence
  Os.RAB17 drought-inducible promoter
cagcggggcagcgcaacacaaaaaggggggaggatgccggcgaccacgctagtgaccatgaagcaagatgatgtgaaagggaggaccggacgagggttggacctctgctgccgacatgaagagcgtgatgtgtagaaggagatgttagaccagatgccgacgcaactagccctggcaaggtcacccgactgatatcgctgcttgcccttgtcctcatgtacacaatcagcttgcttatctctcccatactggtcgtttgtttcccgtggccgaaatagaagaagacagaggtaggttttgttagagaattttagtggtattgtagcctatttgtaattttgttgtactttattgtattaatcaataaaggtgtttcattctattttgactcaatgttgaatccattgatctcttggtgttgcactcagtatgttagaatattacattccgttgaaacaatcttggttaagggttggaacatttttatccgttcgtgaaacatccgtaatattttcgttgaaacaatttttatcgacagcaccgtccaacaatttacaccaatttggacgtgtgatacatagcagtccccaagtgaaactgaccaccagttgaaaggtatacaaagtgaacttattcatctaaaagaccgcagagatgggccgtgggccgtggcctgcgaaacgcagcgttcaggcccatgagcatttattttttaaaaaaatatttcacaacaaaaaagagaacggataaaatccatcgaaaaaaaaaaactttcctacgcatcctctcctatctccatccacggcgagcactcatccaaaccgtccatccacgcgcacagtacacacacatagttatcgtctctccccccgatgagtcaccacccgtgtcttcgagaaacgcctcgcccgacaccgtacgtggcgccaccgccgcgcctgccgcctggacacgtccggctcctctccacgccgcgctggccaccgtccaccggctcccgcacacgtctccctgtctccctccacccatgccgtggcaatcgagctcatctcctcgcctcctccggcttataaatggcggccaccaccttcacctgcttg